SEQUENCE LISTING (79 sequences)

SEQ ID NO: 1 - DNA, 630 bp, Homo sapiens
attgctcaaggtcatgtggctagaatatggcagagccatgattcagatccaggtcttctgattcttattccagtgtcctttctagcataccatgttgcctctaaagattgcagctccttatttactagaaaattgttcctgcccaatctacatctccacctcaccccatcttttcttaagcactatgtttgtgtttttatcagtattatattcattgtctttggaatacatgttcttgtttgtgtttggaaaaaaaatctcttttaccagcttgcactcggaccaacttggaaaaaaaaaagcttaaatgttgttgctatgtacagtttaaaaatgtgaagtttgtagctttaactttttgtaagaaaatctaataacactggcttaagtgctgacttgaaatgctattttgtaaggtttggatgtaagtaatcaattgaggtcagcagtttgtatgagacatagcttcctccattgcccccactccttttttcttttttaagtttgagatgcttcctgtgtttttatgttagaattgttgttctccttcttttcttcttcctatacctcatcacgtttgttttaaataaactgtcctttggaccacaaacccttattaacgagaacctc

SEQ ID NO: 2 - DNA, 1423 bp, Homo sapiens
gctgggagctgacattcaagaaaagtgcattaaaaattccttgagaaggcatattcttttgagagccgtaaatgaaaagtgcattcacggagaacatgctcctttgttgtgagaggaaagaaaggactgttgttgcctttaaggaacaggtttcccagttccccggaatgtcagggtcacatgaggaatgggtgagcacttagggccaagattgtcgtgtgcttggcccggcagcttctctctaagtgacctctccagcatcactctgtgccagtgctcatatctgaggccacttgagtgtcacgaggcagagctgggaaacattagctttggaaaccgttccttcctttttatgtggaggaagtaaggtttcacaagacacctaagctggacatggtgatgtgccacttggatagttcccaaagcagaaagcagccaggacatcccagtaccccacttctccgtgccgtgtacttttagggaggttttcgaaagctagcttcttaccgatgattttgtctatttttctgttttatgtgaaaaccttcatttataacctgctcttctcagtgttcttctattcagcattcagtatgacttctatgcctctaattgcgactgtatggagaagaactgtttgtcattcagtgccgtgggatataatcagtcctgtaaattcatcagaatgttctgttgcctgattttttttccattgcattttaatttcattaaacatattttagttctacagtaacctaattcctcattgttaccatgagatcctaattctgtagctgtgttctgtaatcccatggggcgtgtaccatttggagacacatgaaggatacagtagacagcacggaaggcagcagcaacacacaggggtgtcctccctccccagagcccagtgtgcacacagaaccaaattcaatctcattcatcgtgagcttgcatggtaactgatccagccactcagtacctccattggataagagatttggccacagattcttctagagaaaatccaaatgtggagggtgcatttgtttctgcacccaaacaaataccttttgagatttcttataggcattcctctcgaagtctcattactctgatcagttattgatcggaggctaccctggctcagtcaccagctgctcactgtgtccttctgtggtctcaggagctgcagttgttgctgttgtatgaatagacgatagttaattagactggatgtgtctgtactggcagtgattttgcaacttcctctgtataagcgatacatttgtaaatcatgggaatgtatttgatggtaattgcctgtggtggttgtatcatgatttaattttattatgagcttttggctttatatatttttctgcctttttatgcattcattaaacagtttaacagacaaaaaaac

SEQ ID NO: 3 - DNA, 225 bp, Homo sapiens
gtgcgatgtgttctgagactgaatccagttccaatcttctagatttctttctcgttcttctctgaagatccactattcagaataagactcctgctcatgttaggtgggaatggatacaagggaccatatttggggttctggtagctccacagggatgctcaatgaagatgcaaaattagaagtcaaaataaacagctcccatgggcagtgttgatctcaccctgt

SEQ ID NO: 4 - DNA, 356 bp, Homo sapiens
agtactgaaattaagcagcatccaacacaggcctactcttacgacatgtgactttactgttttccgtttttgttgaaagagtcattaacagttaggagttgatggcagtttcaataacaggtcattgccgagaaaaggatagcactataatatgcagaaatctacaaattctgatacttccgtggaaacactgaattctacccgccaaggcacaggagctgtgcaaatgagaatcaaaaatgccaacagccaccatgacaggctcagccaaagtaaatccatgatcctcaccgatgtcgggaaggtcactgaacctatatccagacacagaaggaatcattcacagcatatcttgt

SEQ ID NO: 5 - DNA, 868 bp, Homo sapiens
  misc_feature (34)..(37): n is selected from the group consisting of a, c, g, and t
cgaacacctgaccttgtgatccacccggcttggnctnccaataatttgtaagttatgttagcgggatcctcaaggccttgctctgccccgtggagacgcttgctcggatgagctcaggaaacagtaccggctgcgtggcaggtctgggtgttgtgtgcgaggacgtggcctttgcacaccgctgtgttctcagaggtccttaggagatatttttttttgtcttagggggactgtgttaagttcagacaaatcatgctgggtgtggagagagtctgaaatacgtcagtgaagtaagtagcagtgagcgattgtgaatgtgtaatgtaaatggaaaaccgggttttaccgtgttaagttattcactagggagccagtcgtagttctttgtaatcctctttcttccaaacctgctttgctgaaagttgcagaaaaggaagtgtgtggagagaaacagaacccttcagggtgggtcagaggacgccatccacagtggattcgtgttcgtttgcaggtggaagcagtgatttttaggacccactgattaaaaacaaacattcccaagtgtctctgagagatgctgtttatttgttaattaaaaagcttttttctctgtcttttaaattatggctttcatgtaataaggatatttttagtgaaaaattgttttcctttcaaattacagaccttttaaaaaaacttaatttgagcgagtaccttttcatttgacacttttcctgtttctaaccttaggaaaccagaatagcgtttggcagacacgacgttttcagtttacctttgacacctgccccactccattttgctttgtgatgtcttcatttaacaataaattatctgaaaaaacaaaaaa

SEQ ID NO: 6 - DNA, 1197 bp, Homo sapiens
gtacatactcattcccaaagcaatccaaaaacaaaatgtgaaccatttgggtttcaaatgttaagaacactaaatagcatgatttaaaaaatgaaaaatgctaacacccaagaaaagaagatattaagtgctttttaacaactcctagagtacaaaatgagtacatcataatgctggctcttctactaatgaaccatcgagtgatattgaataaattatttatcttctcagtttccttatctgtaaattacaatattagactaagtaagtttttccaactcttcactaccaattaccttaggcttttataatgctccgcctacttcagtcccatgtttcagaagcttttgtctattttttaaactcattgattaaataatgattaatgcattctccacattttaatattgcaaaggcccattggagtttctgaagtggctccacagaattgaaataatttcaaataactgtaaaggaactgaaaatcttcacagagatgaagtggggtttccattaggtgctttgaaatttgataacaaatcatcaacttccactggtcaatatatagattttgggtgtctgaggccccaagattagatgccactaatctccaaagattccctccaattatgaaatattttaatgtctacttttagagagcactagccagtatatgaccatgtgattaatttcttttcacactagataaaattacctggttcaaaagtggtttttgtttattaaatttggtaataaatatatataatacacagacaggatagtttttatgctgaagtttttggccagctttagtttgaggactccttgataagcttgctaaactttcagagtgccctgagacacttccagccatccctcctcctgccttcattggggcagacttgcattgcagtctgacagtaattttttttctgattgagaattatgtaaattcaatacaatgtcagtttttaaaagtcaaagttagatcaagagaatatttcagagttttggtttacacatcaagaaacagacacacatacctaggaaagatttacacaatagataatcatcttaatgtgaaagatatttgaagtattaattttaatatattaaatatgatttctgttatagtcttctgtatggaattttgtcacttaagatgagctgcaaataaataataccttcaatggaaaa

SEQ ID NO: 7 - DNA, 521 bp, Homo sapiens
gatggccttgacaccagcagggtgacatccgctattgctacttctctgctcccccacagttcctctggacttctctgggaccacagtcctctgccagacccctgccagaccccagtccaccatgatccatctgggtcacatcctcttcctgcttttgctcccagtggctgcagctcagacgactccaggagagagatcatcactccctgccttttaccctgggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctgaccctcctgcagcttggacctttgacttctgaccctctcatcctggatggtgtgtggtggcacaggaacccccgccccaacttttggattgtaataaaacaattgaaacctcgagccg

SEQ ID NO: 8 - DNA, 584 bp, Homo sapiens
cacttgcctggacgctgcgccacatcccaccggcccttacactgtggtgtccagcagcatccgggcttcatggggggacttgaaccctgcagcaggctcctgctcctgcctgctcctgctggctgtaagtggtctccgtcctgtccagggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatgagcccgaatcatgacagtcagcaacatgatacctggatccagccattcctgaagcccaccctgcacctcattccaactcctaccgcgatacagacccacagagtgccatccctgagagaccagaccgctccccaatactctcctaaaataaacatgaagcacaaaaaaaaaa

SEQ ID NO: 9 - DNA, 588 bp, Homo sapiens
  misc_feature (273)..(306): n is defined as selected from the group consisting of a, c, g, and t
gcaaggcggcccaggacaggcaggggctgcacgcggtgaagaaaccaagacgcagagaggccaagccccttgccttgggtcacacagccaaaggaggcagagccagaactcacaaccagatccagaggcaacagggacatggccacctgggacgaaaaggcagtcacccgcagggccaaggtggctcccgctgagaggatgagcaagttcttaaggcacttcacggtcgtgggagacgactaccatgcctggaacatcaactacaagaaatgnnnnnnnnnnnnnnnnnnnnnnnnnnnnnnnnnnccaccacccacaccagtctcaggcgaggaaggcagagctgcagcccctgacgttgcccctgcccctggccccgcacccagggccccccttgacttcaggggcatgttgaggaaactgttcagctcccacaggtttcaggtcatcatcatctgcttggtggttctggatgccctcctggtgcttgctgagctcatcctggacctgaagatcatccagcccgacaagaataactatgctgccatggtattccactacatgagcatcaccatcttggtcttttta

SEQ ID NO: 10 - DNA, 797 bp, Homo sapiens
cccaatcctttggcaggcatgcagctccacaggcgatttcttcaagcagctgcagtgtttagccctcctgggttaagagccagataaggagaaatccctttcctaggtttggaatgtgttgtgaaaaaaaagagaaatccctggctcctggagctggtgggagacaagattaagcaaacctcccctgacatgtatccctttgaccccaagctctgcctcctccctgaccacccatgccctttcctttaacttctcaaacagataccagggcctaaactgctttacctcccctcctactgagtcaggttaggtggtgggaggtcacccatttccgagttaaaccaatgcaatatgagtaaaacaaagtcatgtgggtatgtctggggtagagagaggggtagcaagttcatgtgtcctccttggtcacatatctcccaaagctctgatccctgccatgggaagtggacaggaaacatgaggtcatgacctgcaggcatctttactgcagctctgccggcctggagggggagagggggaggaagaagtatgcgctgcacatttctgaggctactgcatttgctttcaaggcagaaatcttgctctgagcagtcagcggctccagtttgggcccgataaggaagttctccgtggcctccctcaggcagagcagggaggaggctgacattgccagtctcttctggggcccaaggcaggttgcaggagatccaatcccatagacagctctgggcctcttgcatttgagtttttcagaattaaactgcagtattttggaaagc

SEQ ID NO: 11 - DNA, 239 bp, Homo sapiens
gaaatgtcagtggaagaagcagatgagaaactcttgagatcttggtcctgtgttttttctgccaccaaaggccagggtcactgaaggcctggcccacagcaggtgctgagcaaagggaacagtgaggtgcccagctagctgcagagccaccctgtgttgacacctcgcccctgctccctcccatcccttccccctttactcatagcacttcccccattggacacgtggtgcattttgct

SEQ ID NO: 12 - DNA, 665 bp, Homo sapiens
acagcagccaccgcccagttccagtggccccacgaagcccccagtggctggctgtccagctgggcaaacagtggcacccctcccagctcttctgagtggggagtcttccaggcctcctcagaggtcttccctttgcctccccaggacagggtgagtcagagctcagcatttaatctcctctccaaagtagagagcaagtcgcccacagtgggcgtgtctgtaaatattgtgacagtatttttttactgtgctgttttttttgaaaggggatgggtaaaaagtagggtgttcttgttttttgcttgggagggtgggggtgggggagggtcttattttatcttatcttttctgtggatcagaaaaaacagaagccaaactcggggtcatctttgtttttaaagctgaagtgggactgtctggcactctgtgtatttatgcgttccagcatctggaacctcccatccctgccctcctcctgtgtagctgccacctccccgctgggcccagcatggctcacctgtcccgtgggctgtgtttcttgttgtttttctctttgcaaagacatagctaggaaagcgaatgataagggaaaagttctcagggaattgaagtgttgttgctatggtgacgtccttttgctgtgaataaaggtgctctttgcagca

SEQ ID NO: 13 - DNA, 1498 bp, Homo sapiens
atagatttggcctagcctcttctgttaacctagtccacagatgagcgaatctggttagttgaaggacattgtgatttgactctggtcacgcgaggaagtagaagggcaaagacaggaccggcagtttacatttccagtggttaaacctcacggtactttgggactgcttgttaacttttgtggttgtctgaggccaatctaacgtgaccatttctgacacctcaacagagagaggaaagcaacttgagcaatgagagtaaataacttgggctctcagagatttgaagatagagatctcattgtgagggggactattttgcaggtcctcatttctccaagaaagagatggtgttacaggaacccactgaaagccatatcccattaaatgaggaactaattttggctgggccttcttgtaatgtcctcgcaggtgtgttgtgaagattaatgcagggtagtatgtttgtagattgacacctagtctaaacttgaggtaattggtgctctgtgaatactcagtcgtgttcttttatagccttaatcatgatttgaactagtcccttgctttttaaatgactgaatgaagtccttcgtggtaagggagtacgttgataacttagtttactatatgggtttgtggtcgcatcccagtcatcagctgctatcattttccttcttcatcccttatactgagatttgggttacagctttttattcttcgaaggatcacaaagcagtgtacagacacctgccttctttaaggatgaaaggaagataaagtggtctttttttgtttacttatttgtttcacctcttgtttgagtaacttctaaggtgctattctctctctctttttgctaccccatgagctcttgtcacagccatggaaaccagcctcgtttagaaagggaacttagttcagaaggggttaaaagccttccagaatttttctttagctgctgaagtttttacatgtggttacatgactttaagttttatgcattacgctcttaattctattacaaaatgtggactcaccaattgctttgtgttttccatgtgacctgttacttcaggctacttggggaacatcttagtcctctgtagctcctgaacccagcactggtgcttcaagagagaaggtagcacgtctttgttcaaaacaaaacaaaacgacacttctggaggccacatcctgaatatgaatgttctactaagtcactcagttatggttctaaagggaaactgtaagaagacccacaaggagtggaccaagactattatttaattgcacaacttgaaactttgctgccagaagaggcagctccattcctttgactccagtgttgggctgttaactgctgcacctcattgcctttttttgtttttgtttttgttttgtaggagggtaggcactgttgggccatatgcacaaatattgtaactcttggtatctttactgcatcatagtcaataaacttctttgtaccctttgaaa

SEQ ID NO: 14 - DNA, 644 bp, Homo sapiens
ctcctggttcatccaggcacctgcctataccccacatcccttctgcctcgaggccaagatgcctaaaaaagctaaaggccaccccaccccccacccaccacctcctgcctcctctcctctttggggatcaccagctctgactccaccaaccctcatccaggaatctgccatgagtcccagggagtcacactccccactcccttcctggcttgtatttacttttcttggccctggccagggctgggcgcaaggcacgcagtgatgggcaaaccaattgctgcccatctggcctgtgtgcccatctttttctggagaaagtcagattcacagcatgacagagatttgacaccagggagatcctccatagctggctttgaggacacggggaccacagccatgagcggcctctaagagctgagagacagccggcagggaatcggaaccctcagacccacagccgcaagggactggattctggcagcaccctgaaggagctgggaagtaagttcttccccagcctccagataagagccccgccggccaatcccttcatttcaacctaaagagaccctaagcagagaacctagctgagccactcctgacctacaaagttgtgacttaataaatgtgtgctttaagctgca

SEQ ID NO: 15 - DNA, 418 bp, Homo sapiens
ctctcatttctttgccttcagcagaatcatcttaaaacctgccaaacttatccttccttcacagctttgcttttctgcctcttctctcaagcctgcttcagatcataagttcttccacacatctcctgaatcactccaaacccgcatttacctttttattttctgatataagctttgatgcctcttcaattcttaggacatttaaacatatgaatgttgccacagcattttattacctagcttcatatgaaaatgtcttaaattcccacctaaatgaaaagaaactgcccaaatgcctagaacatcacataaggcactaaatgcctcatgttttactgacgggaattgaattgtacattttgctgagtagttttgagaaaaaaatctaataaattcatctgttattcatccataaacg

SEQ ID NO: 16 - DNA, 388 bp, Homo sapiens
aaaagtcttcggtcgttagaagttgaatgggcacagcaactctaagactacagcacacgtcatttcttagctaagcggaccagcctccctgtcggcctggtgttctgtgggatccctctgggcactggtaatcccaagatctgtgcagccccgcctccaggccacatggggctgggcagctaccatttcccttttgcggatgggaggggtaacttgcacctctgacctatcacttccactgcaccccgtctcattcctccacctgccgtggacttggggtcagagactgctgtgtttgagctctgcagcccagggaccgaaaagttggtgtcaatgaattttgcttggtggatgaaatgtcagtggaagaagcagatgagaaactctt

SEQ ID NO: 17 - DNA, 684 bp, Homo sapiens
gtgaaaaagaggaacaagttctaacccaatgcaggtttaaaacataaccaaaagaacaaaagagagagcttgacacaccaatcttgaggtgttgtgccattctctgggaacatacagatgaaatgacctgaaccctgcccaccatttaaccaagtaagaatacaattctctctcatttaggttatcacgctataatagagaatggactcattaatatgggattacagaggaaggaatggttaagaaaaagacatggcctctgactgctccaaaaaaggataagcagggcttcacagaagaggtgacagaaaaggaagtggaatggaataaaatcccccaatacagtacaattatacattaatggctgtaatgtgaagagcatcacacacgaagagagccatcttccagaaataagtttatacactctctcctctaattgcatcaggactttaccagataatgttcttccagatctgaaaaggaaaatgcctaaaagagcttccaaactcatttttgaataatactaggctacaaagaattacactgtgaattcattaagggtaacaccaaaccactaaacagcactgtttgtacagaaatgtcgaaaagctgtggaaataatttagcggccatttctgtaggaatttcgcttcttttactcttagtggtttgtggaattgggtg

SEQ ID NO: 18 - DNA, 376 bp, Homo sapiens
gtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagttt

SEQ ID NO: 19 - DNA, 1654 bp, Homo sapiens
tgcgcctttcagcctcacctgcagctgcgcctccttgcacctgcgcctgtgctttttctcccagcactgcggacgcgactcgagggtgacgctcgctccgctcgtcccgctcgtcatggcctacccgggatacggaggagggtttggaaattttagcattcaggtgccaggaatgcagatgggacagccagtgccagaaacaggcccagctatactcctcgatggatactctgggccagcatattcagacacttattcctcagctggtgactccgtgtatacttacttcagtgctgttgctggacaggatggtgaagtggatgctgaagaacttcagagatgtttgacacagtctggaattaatggaacttactctcccttcagtttggaaacctgcagaattatgattgccatgttggatagagatcacacaggaaaaatgggatttaatgcattcaaagagctatgggcagctcttaatgcctggaaggaaaacttcatgactgttgatcaagatggaagtggcacagtagaacatcatgagttgcgtcaagccattggtcttatgggttataggttgagtcctcaaacattaactactattgttaaacgttatagcaagaatggcagaattttctttgatgattatgttgcttgctgtgtgaagcttcgagcattgacagatttctttaggaaaagagaccacttgcaacaagggtctgcgaatttcatatatgacgattttttgcagggcactatggcaatttgaatgcttagaattttaaacctgaagagacactgtggaattcttttgtttggaagaagtgaactgggactactttaaaaacttttaagggttttctatgttcttcctacctgttaaacctcttccctttctgtgtgtttttattttagcagatagttcaaagcaataaaagatttcttttttaatttgaggtattactgcttttggaaaagttattttataaatatgtgcatattgtcataaaatattgtatgattaattgatttaaataatgcttagccttaattttagataatgtaaatttagaggaatgtactttacaagatagattgtataagaagccaaataatgaaagcctagaaaaaactaatttatacttatctgaaggttacaaattagacttttaaattttctttgtagttggtggtgtttgagggttggctagaaatgaaagcctggattttgtgccatgtttgtaatatagtttgttccttgatcaaataatcagagaaaagaaacttaaagatctttgtctgtgaagaagaaaattatctccctagttcaatctgtagtgaaataagactacagaaggcattgttttttccttttttattttttgtattatatatttttcttaaatatgttttattgtcttctctaagcaaaaagttcttaataaacatagtatttctctctgcgtcctatttcattagtgaagacatagttcacctaaaatggcatcctgctctgaatctagactttttagaaatggcatatgtttttgatgatatgtcaacattcaaaattgtcctaattaaattgttgtttaaatgtaatgtcaactctttataaacttaaaaataaacaagtaattaaccactctaaaaa

SEQ ID NO: 20 - DNA, 1520 bp, Homo sapiens
cgcactccctgctggggtgagcagcactgtaaagatgaagctggctaactggtactggctgagctcagctgttcttgccacttacggttttttggttgtggcaaacaatgaaacagaggaaattaaagatgaaagagcaaaggatgtctgcccagtgagactagaaagcagagggaaatgcgaagaggcaggggagtgcccctaccaggtaagcctgccccccttgactattcagctcccgaagcaattcagcaggatcgaggaggtgttcaaagaagtccaaaacctcaaggaaatcgtaaatagtctaaagaaatcttgccaagactgcaagctgcaggctgatgacaacggagacccaggcagaaacggactgttgttacccagtacaggagccccgggagaggttggtgataacagagttagagaattagagagtgaggttaacaagctgtcctctgagctaaagaatgccaaagaggagatcaatgtacttcatggtcgcctggagaagctgaatcttgtaaatatgaacaacatagaaaattatgttgacagcaaagtggcaaatctaacatttgttgtcaatagtttggatggcaaatgttcaaagtgtcccagccaagaacaaatacagtcacgtccagttcaacatctaatatataaagattgctctgactactacgcaataggcaaaagaagcagtgagacctacagagttacacctgatcccaaaaatagtagctttgaagtttactgtgacatggagaccatggggggaggctggacagtgctgcaggcacgtctcgatgggagcaccaacttcaccagaacatggcaagactacaaagcaggctttggaaacctcagaagggaattttggctggggaacgataaaattcatcttctgaccaagagtaaggaaatgattctgagaatagatcttgaagactttaatggtgtcgaactatatgccttgtatgatcagttttatgtggctaatgagtttctcaaatatcgtttacacgttggtaactataatggcacagctggagcatgcattacgtttcaacaaacattacaaccacgatctgaagtttttcaccactccagataaagacaatgatcgatatccttctgggaactgtgggctgtactacagttcaggctggtggtttgatgcatgtctttctgcaaacttaaatggcaaatattatcaccaaaaatacagaggtgtccgtaatgggattttctggggtacctggcctggtgtaagtgaggcacaccctggtggctacaagtcctccttcaaagaggctaagatgatgatcagacccaagcactttaagccataaatcactctgttcattcctccaggtattcgttatctaatagggcaattaattccttcagcactttagaatatgccttgtttcatatttttcatagctaaaaaatgatgtctgacggctaggttcttatgctacacagcatttgaaataaagctgaaaaacaatgca

SEQ ID NO: 21 - DNA, 112 bp, Homo sapiens
ccggccatgattttgaggactccccagccatgctgaactaaactcggaccccaccaaatggatctgctggcacacagacctcagatatggagggaccgagaggactggattc

SEQ ID NO: 22 - DNA, 2126 bp, Homo sapiens
  misc_feature (119)..(119): n is defined as selected from the group consisting of a, c, g, and t
aaaaaaaaagacgaactattggaggtggtggccaatgatgcatttactgtttgcaggatagttaaaggtgtttaaagggtaaggcttttggtgtaaatgctggatggggtgtgtgtgtntgtggatatagggacctccctctgtactgtgtactgtgtaatcggcattaatacctagactcatatgtatggaattttaaattctcttagcctactgattggtttggatgagcacaccagctgcaggtgtgtgctgaattgcaagatggtatttttttttttaaccaagggatgtctcttgtaatactaaccgcgtgataatgggttttcagacatgatgaaaaaaaaaaacttttacaaatgaatacttaccttagaaatattcaccttaggaaaaaagactttgctctgcccttttatattcctttatgctgcaagtggtgacatgttcagatttctaatttggttcattgtggcctatctggtttaagtctttcattaaaaatgtctcgttagagtatttgatgtcatgcaccaaaaaaataaaaccccaccttgttgcaaaagctgacctcgttgcatggaattaaaagagaaggaaaaacacaaggatgaagtctttccgaattcattcttgtgggaactggccttcggagcccagccagcactttgggcaaatgcaaacaacaatgagtgcttgagataaaagaaagtgtgacgtcatggtcactggtactcaggcacttcacagtttacttgaaagaggctttggaaaatagataaagtgaaagaagaataaatacatatttttaataatgtaattttaaaaatcctttataatcaggactgagtcttggtttgcagaagctgtcacttaccctgaaacacagtatcaaaagggaaacttaaaacatactgtttgatttttttatttcctcttacaatccatgttttcaggtagaattatgactttccccccattgttacacatttctttacaaaggaggcctgtagaaattggacacgatcatgcttgagcatgtgagttagtcaaattatgagtccctgcctattgtccattacacaccgaatgttaatttaagaaccagaggcagaagttctggcttcctgcttgaaacccaattcttatatgaaattttttaaaagcagaaacctagcagcccatctgctttttctcttttgtcggtgtatttggtacccctccaatgctggtctttttgtagaaactcagtagagaaagtctagctaagcagtgttgaaaagcctgcaagatttcagtttacatatcgacagcatatccactgatttctaaatgggctggtcccatcatctgaagattctgtatagaattattaaaaaaaaaaatccatctttctttattttcttcacatgcgacaatttcttaagcactttgacattttggtagttccacactattgagagaataatatatttattttgtgacattggcagatgccaaatactgtaaccttctcgtgataacaatacttaggttcaagatcactgttcaaaccctgtcatgctttaaaactgatgcgagatgattttgttttttgcataatcaatacttaagggtgcaatcaactgttagtaattgtgcagtaaagtaaagccctgtggtgtatcaactactagttaagagtctcagttgatttctgtaatgtttgacctaataatagcccgtttcgtctctgacccaacagaggaagcacagatcaaatcaccttggagtggtcaccagggggacagggagccccccaccaatgtatcaatgggtgatttatgatgccttctgccctttggcgagtgaatgggtttcccataggggaagttggcctccctccgtgagctttggaaatgttttctaatagacacagggaggccagttctgtttcagagcaattatcttcccaaattctctgttctggtgttggaactgtgtgccctggtttctgttttcctttctactgctgtaattctctgtctcatcatccttctcttttgtttccatagccttttataatgcatatatgatgctgtgaacagaaataaattatttatacaatcaaaaaaaaaaaaaaaaaa

SEQ ID NO: 23 - DNA, 1851 bp, Homo sapiens
ggcagactcccagaaaaggagagcccgagagggacaagccaggagctgagagtcaccggaagaaccgcagcattgtcaccaacatggacaagctacacctaaacttgacagaactggcactgacaatgaatcatgtatacagtttctccgtgtttgaacatactatcttcccttctgagtacctcagcagccacctggaggccagactcaacagagccattgtgtggctggctggctacaatgccacgacccaggagatcgtacggccttctgagctgttggcaggagtcaaagcatacattggtttcatacagtcactggcccagtttttgggtgcagatgcttccagagtcatccgcaacgccctcctgcagcagacacaaccactggattcctgtggggaacagacaatcaccacactctacacaaactggtacctggaaagtctgcttagacaggcaagcagtgggaccatcatcctctccccagccatgcaggccttcgtcagcctgcccagagaaggggagcagaacttcagtgcagaggagttctctgacatctctgagatgcgggccttggcagaactcctgggcccctatggcatgaagttcctgagtgaaaacctgatgtggcatgtgacctctcagattgtggagctgaagaagctggtggtggaaaacatggacatacttgttcagatcagatccaactttagcaagccggacttgatggcttccctgctgccccagctgacaggggctgaaaatgtgctaaagcgcatgaccatcattggggttatcctcagtttcagggccatggcccaagagggacttcgggaggttttctcctcccactgcccatttcttatgggtcccattgagtgcttgaaggagtttgtcactccagacacagacatcaaggtgaccttggagtatctttgagctggcatctgctgcaggtgtgggctgtgacattgacccagccttggtggctgccattgctaatctgaaagctgatacttcatctcctgaggaggaatataaggtggcctgcctgctcttgatctttctggcagtttccctcccactccttgccactgacccttcttccttttatagcattgagaaggatggttacaacaacaatattcattgcttgaccaaagccatcatccaggtgtctgctgccctcttcacgctctacaacaagaacattgaaactcacctcaaggaatttctggtggtggcctctgtcagcctcttgcagctgggccaggagactgacaagcttaaaaccagaaatcgagaatccatttctctgctcatgcgcttggtggtggaggagtcatccttcctgaccctggacatgctggagtcctgtttcccttatgtcctgcttcgaaatgcctatcgggaggtgtctcgggccttccacctaaactgaatgcctgccagtacccactgaagagccctttggaccttcctaaacccttgccatagtggaagctgtggtcactttcgcagggggtgggaatggggtggggtcactaaggagagagggtcaggagccagagttgatgagcagatctgtggaagaacaatccagggctgagaaatcgtagagcagtgaggcaggctgggagcatggaggacagcttatggaaaaagttagggcgtggggccacatgtgtgaattttacaatgaaaaaaggagtaacgtacaagtatattttctatcttctggtgacttgagcttgagctctgacaggcatgggcctctccgaccttcatcactattcttaggataatgctggcgggcagagatgatcaatcatcatattaaatcataatg

SEQ ID NO: 24 - DNA, 337 bp, Homo sapiens
ctctcccacctctgtctgcccgctgcctcttgtctagctgctgtcaggagctgactgcctccagggctggaatcctgtgctccctctgtgcccagagccccacgatgtcggccaacgccacactgaagccactctgccccatcctggagcagatgagccgtctccagagccacagcaacaccagcatccgctacatcgaccacgcggccgtgctgctgcacggggctggcctcgctgctgggcctggtggagaatggagtcatcctcttcgtggtgggctgccgcatgcgccagaccgtggtcaacaactgggtgctgcaactgggggtgtccgaac

SEQ ID NO: 25 - DNA, 431 bp, Homo sapiens
gtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaat

SEQ ID NO: 26 - DNA, 466 bp, Homo sapiens
gcactggaaacaccgtgttgccacacgatttaccttaccgaggtttttacaaaggagaagcagcaggagaaaagtctgtactaaaacattcttgggcccccgcatcattggcttaaggcatgaaatctcagttgaaacccaagaccacaaatctgctgtcaggggaaataacacacacgacaactatgaaaatgtggaagcaggtcctcccaaagctaaaggaaaaaccgataaggaactatatgaaaacacagggcagtctaatttcgaggagcatatctatggaaatgagacatcttctgactattataacttccagaagcctcgtccttctgaagttcctcaagatgaagatatatacattcttccagattcatattagcttttcaaaatattgacttttgttattggatgataaatattcactgtaatttttcaacagcaaagacaaggaatcaaactaaat

SEQ ID NO: 27 - DNA, 508 bp, Homo sapiens
gccaggacccagacagagacacacggtcactgcagctgaagccgctgcccctgctacagatgaagaaaaggaggctgggagagccaaggatcacgcagcaaggaagaaacagacccaggtgcagactcaggcaccaccaggaccagctgatcattccagcccacagcaatggagccacatgactcctcccacatggactctgagttccgatacactctcttcccgattgtttacagcatcatctttgtgctcggggtcattgctaatggctacgtgctgtgggtctttgcccgcctgtacccttgcaagaaattcaatgagataaagatcttcatggtgaacctcaccatggcggacatgctcttcttgatcaccctgccactttggattgtctactaccaaaaccagggcaactggatactccccaaattcctgtgcaacgtggctggctgccttttcttcatcaacacctactgctctgtggccttcctgggcgtcatcacttata

SEQ ID NO: 28 - DNA, 239 bp, Homo sapiens
ggtatacaacatgaaatatctcaatcccacttcagatttctaattgtttctgcttccagaggagaagccaagtcaaaatgtcctgaataagcagttctctattgtgagaggcctcttgtggaatctgggattgaaacaattctaaatgccccacttctttcatgcatgaattgcaaaaagatgtggcaagttttgtttctaccaagaaaactaaaaacaccttttgtcaaataaatgct

SEQ ID NO: 29 - DNA, 1556 bp, Homo sapiens
aaaaggaggtctgcacagcctctcttaaaacaacatgccacagttaaaacttacttcagtttggacaactactcacagctactacacagagacccgaacgagtcactgatatacacctggaccaccaccaatggatatacaaatggcaaacaattttactccgccctctgcaactcctcagggaaatgactgtgacctctatgcacatcacagcacggccaggatagtaatgcctctgcattacagcctcgtcttcatcattgggctcgtgggaaacttactagccttggtcgtcattgttcaaaacaggaaaaaaatcaactctaccaccctctattcaacaaatttggtgatttctgatatactttttaccaccgctttggcctacacgaatagcctactatgcaatgggctttgactggagaatcggagatgccttgtgtaggataactgcgctagtgttttacatcaacacatatgcaggtgtgaactttatgacctgcctgagtattgaccgcttcattgctgtggtgcaccctctacgctacaacaagataaaaaggattgaacatgcaaaaggcgtgtgcatatttgtctggattctagtatttgctcagacactcccactcctcatcaaccctatgtcaaagcaggaggctgaaaggattacatgcatggagtatccaaactttgaagaaactaaatctcttccctggattctgcttggggcatgtttcataggatatgtacttccacttataatcattctcatctgctattctcagatctgctgcaaactcttcagaactgccaaacaaaacccactcactgagaaatctggtgtaaacaaaaaggctctcaacacaattattcttattattgttgtgtttgttctctgtttcacaccttaccatgttgcaattattcaacatatgattaagaagcttcgtttctctaatttcctggaatgtagccaaagacattcgttccagatttctctgcactttacagtatgcctgatgaacttcaattgctgcatggacccttttatctacttctttgcatgtaaagggtataagagaaaggttatgaggatgctgaaacggcaagtcagtgtatcgatttctagtgctgtgaagtcagcccctgaagaaaattcacgtgaaatgacagaaacgcagatgatgatacattccaagtcttcaaatggaaagtgaaatggattgtattttggtttatagtgacgtaaactgtatgacaaactttgcaggacttcccttataaagcaaaataattgttcagcttccaattagtattcttttatatttctttcattgggcactttcccatctccaactcggaagtaagcccaagagaacaacataaagcaaacaacataaagcacaataaaaatgcaaataaatatttcatttttatttgtaaacgaatacaccaaaaggaggcgctcttaataactcccaatgtaaaaagttttgttttaataaaaaatttaattattatttcttgccaacaaatggctagaaaggactgaatag

SEQ ID NO: 30 - DNA, 457 bp, Homo sapiens
ggggcgatgaggtgaggacgcccgggaaccggaggcggcaccgcgcggcgcacggacctgggacgcggagtcctgaagccggcggacggttttcgtacgggcggccgtgcgcgaggcgaggagagaacattgaaagtattctctaagctatttgaagagagtgactaaatgcacctgggtcaggctgtctgtgggtatgaagtggttgggagaatccaagaacatggtggtgaatggcaggagaaatggaggcaagttgtctaatgaccatcagcagaatcaatcaaaattacagcacacggggaaggacaccctgaaggctggcaaaaatgcagtcgagaggaggtcgaacagatgtaatggtaactcgggatttgaaggacagagtcgctatgtaccatcctctggaatgtccgccgaagaactctgtgaaaatgatgacctagaaccagtttgg

SEQ ID NO: 31 - DNA, 885 bp, Homo sapiens
ggcgagaagtgtgaggccgcggtagggccgcatcccgctccggagagaagtctgagtccgccaggctctgcaggcccgcggaagctcgacagcgtcatgggcagagcaggtggccctgagccggacccaggtgtgcgggatccctgcgggaagagcttttccaggggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaac

SEQ ID NO: 32 - DNA, 2781 bp, Homo sapiens
tcagcatgcagaggaggcccagctgctgagaggagttgcctgagagtgacctttgcatctgcctgtccagccagcatggaaccaaagcggatcagagagggctaccttgtgaagaaggggagcgtgttcaatacgtggaaacccatgtgggttgtattgttagaagatggaattgaattctataagaagaaaagtgacaacagccccaaaggaatgatcccgctgaaagggagcactctgactagcccttgtcaagactttggcaaaaggatgtttgtgtttaagatcactacgaccaaacagcaggaccacttcttccaggcagccttcctggaggagagagatgcctgggttcgggatatcaataaggccattaaatgcattgaaggaggccagaaatttgccaggaaatctaccaggaggtccattcgactgccagaaaccattgacttaggtgccttatatttgtccatgaaagacactgaaaaaggaataaaagaactgaatctagagaaggacaagaagatttttaatcactgcttcacaggtaactgcgtcattgattggctggtatccaaccagtctgttaggaatcgccaggaaggcctcatgattgcttcatcgctgctcaatgaggggtatctgcagcctgctggagacatgtccaagagtgcagtggatggaactgctgaaaaccctttcctggacaaccctgatgccttctactactttccagacagtgggttcttctgtgaagagaattccagtgatgatgatgtgattctgaaagaagaattcagaggggtcattatcaagcagggatgtttactgaagcaggggcatagaaggaaaaactggaaagtgaggaagttcatcttgagagaagaccctgcctacctgcactactatgaccctgctggggcagaagatcccctgggagcaattcacttgagaggctgtgtggtgacttcagtggagagcaactcaaatggcaggaagagtgaggaagagaacctttttgagatcatcacagcagatgaagtgcactatttcttgcaagcagccacccccaaggagcgcacagagtggatcaaagccatccagatggcctcccgaactgggaagtaaagacactcctgcattcctcctcccctcctgagggaagcccatggacaagctcagtccaggacctgtccacttctgtgacaaatcaacgggaaacagcccaggggtgggaagttttcatttgcaggggggtctgaatgtaactcaccatgtggtgtgcaaggttcccctgcattgtattgctcactgcagcccctctgcccctatccatgacccccaagcagatataacaagctgtgcagcctcagtaggctgcttgccctctccaggcctcagggcctcttctggaaaatgaagaaattcaactagtagattcctgaggtctccccagcttaaaaaaaaaaaaaatctgccccatgattctaacactcgcagtagtgatagtgtatctagttgttctgctggtgtccttccttggctaagtcttggccttcagttatcttcaaatgtaccagaacctgagccaacgcctccctgtgaaactgttgctgatctgtagtacagtaccaggaagaaacctcttttgttctctttagacatcttctacttgctcttggccttgagatcgtgtaacaaaatgaaggagggctctcttctttcttcctcatcctactcaaaaacttcccgagagcagtggtggttttgagggttttgacttctattacttttgggcagcctgggaaagttgtgtcttctggggaaagagacccggggaggccagggagtagctgagggtcctttctgtgcccttaaaccgcccagagggagccctattccactctggttttaggctgatctgaggagggtctccctttgttcctttctggagcatttctctaacgtttattacaattaggagggggaccccacatctgtgagattctgtttcatttgaggtttacagaaaaaaaaaagtggccagatgtgttccccccatgggtgagaggcctgggcaactgcctggtgaatgtgtctcgcggcagctgcagcaagtggaggggctgaactactggccagctcactgggatgatgggttaatacaacaactgcactgtaaggactcagagccacacagaacttctgagaggggctgttagcattgcgcagattcttcagttctccagtaaatgatattgcgttcgtgcctcagctttaagcacaagtagcagcagctcctgcttgagttctgagggcatcatggccctatgattaaccagagtgatctaacctagactaaaattgggaacttatttgcaatttttgaccctgaccactaactagtgattcttctccaaaattgagaaagacagcacccattgaaacagatatgtgtgtgaaagtatatttttcaattccagatttttaattttaaggctccaggaaagaaaggagagtgaacatttttcctcattttatcaaatcctctcttgccctccctcaattcccctgtaacattcctgaagctgttcccactcccagatggttttatcaatagcctagaggtaaagaactgtctttttctctgattctttaataaattatctttatagaatatgcacaagtttttctacactcagtgttaaagtatttattaatgggaagtcaacttaatgttttgaaataaatatatgactctgtttaatgcaaaaaaaaaaa

SEQ ID NO: 33 - DNA, 445 bp, Homo sapiens
ctgcaagagtggggcagagaaccagagtgtcagagcaaaacctcctctatctgcacatcctggggacgaaccgggcagccggagagctgcggccggcccagtcccgctccgcctttgaagggtaaaacccaaggcggggccttggttctggcagaagggacgctatgaccgcagaattcctctccctgctttgcctcgggctgtgtctgggctacgaagatgagaaaaagaatgagaaaccgcccaagccctccctccacgcctggcccagctcggtggttgaagccgagagcaatgtgaccctgaagtgtcaggctcattcccagaatgtgacatttgtgctgcgcaaggtgaacgactctgggtacaagcaggaacagagctcggcagaaaacgaagctgaattccccttcacggacctgaagcctaaggatgctgggaggta

SEQ ID NO: 34 - DNA, 664 bp, Homo sapiens
tgggaggctcctctcctagaccctgcatcctgaaagctgcgtacctgagagcctgcggtctggctgcagggacacacccaaggggaggagctgcaatcgtgtctggggccccagcccaggctggccggagctcctgtttcccgctgctctgctgcctgcccggggtaccaacatggcccagaagcgtcctgcctgcaccctgaagcctgagtgtgtccagcagctgctggtttgctcccaggaggccaagaagtcagcctactgcccctacagtcactttcctgtgggggctgccctgctcacccaggaggggagaatcttcaaagtttggcaccaactggcccgtgtacatgaccaagccggatggtacgtatattgtcatgacggtccaggagctgctgccctcctcctttgggcctgaggacctgcagaagactcagtgacagccagagaatgcccactgcctgtaacagccacctggagaacttcataaagatgtctcacagccctggggacacctgcccagtgggccccagccctacagggactgggcaaagatgatgtttccagattacactccagcctgagtcagcacccctcctagcaacctgccttgggacttagaacaccgccgccccctgccccacctttcctttccttcctg

SEQ ID NO: 35 - DNA, 1615 bp, Homo sapiens
aaggcatatatctgcgtatgtgtggtacttagtcacatctttgtcaacaaactgttcgtttttaagttacaaatttgaatttaatgttgtcatcatcgtcatgtgtttccccaaagggaagccagtcattgaccatttaaaaagtctcctgctaagtatggaaatcagacagtaagagaaagccaaaaagcaatgcagagaaaggtgtccaagctgtcttcagccttccccagctaaagagcagaggagggcctgggctacttgggttccccatcggcctccagcactgcctccctcctcccactgcgactctgggatctccaggtgctgcccaaggagttgccttgattacagagaggggagcctccaattcggccaacttggagtcctttctgttttgaagcatgggccagacccggcactgcgctcggagagccggtgggcctggcctccccgtcgacctcagtgcctttttgttttcagagagaaataggagtagggcgagtttgcctgaagctctgctgctggcttctcctgccaggaagtgaacaatggcggcggtgtgggagacaaggccaggagagcccgcgttcagtatgggttgagggtcacagacctccctcccatctgggtgcctgagttttgactccaatcagtgataccagaccacattgacagggaggatcaaattcctgacttacatttgcactggcttcttgtttaggctgaatcctaaaataaattagtcaaaaaattccaacaagtagccaggactgcagagacactccagtgcagagggagaaggacttgtaattttcaaaggcagggctggttttccaacccagcctctgagaaaccatttctttgctatcctctgccttcccaagtccctcttgggtcggttcaagcccaagcttgttcgtgtagcttcagaagttccctctccgacccaggctgagtccatactgcccctgatcccagaaggaatgctgacccctcgtcgtatgaactgtgcatagtctccagagcttcaaaggcaacacaagctcgcaactctaagatttttttaaaccacaaaaaccctggttagccatctcatgctcagccttatcacttccctccctttagaaactctctccctgctgtatattaaagggagcaggtggagagtcattttccttcgtcctgcatgtctctaacattaatagaaggcatggctcctgctgcaaccgctgtgaatgctgctgagaacctccctctatggggatggctattttatttttgagaaggaaaaaaaaagtcatgtatatatacacataaaggcatatagctatatataaagagataagggtgtttatgaaatgagaaaattattggacaattcagactttactaaagcacagttagacccaaggcctatgctgaggtctaaacctctgaaaaaagtatagtatcgagtacccgttccctcccagaggtgggagtaactgctggtagtgccttctttggttgtgttgctcagtgtgtaagtgtttgtttccaggatattttctttttaaatgtctttcttatatgggttttaaaaaaaagtaataaaagcctgttgcaaaaatga

SEQ ID NO: 36 - DNA, 1063 bp, Homo sapiens
gctacttgcatcgacttgtacctcacttagcccttgggggcgtcgtgagcttggattgtttaaggagggctcaggggtaggaatcgcgatggctttataacaatacttgaaaactaacgacacgcatacattttcttattttctggtggaggagcttagtaagtggtgctacaattgctgtgcaaagaaattccagaggggagaagaatgtaaaagtttggtggtgggtggcttggcattgcccctttttcccaccgattcggtggctggtgaaggtgggagatgtgaactccaattaagggactggagagaggtgaagaattttgcaggtgggagatttggatttgaatgtggacttgtaaatgacttgaccttgccatctgtgttcaaggtcacggtttgctgtggggttcctgggagagcttactcaccccggagtcttttctttctcttgctccaagaagagccctgttggtgctttaccaccgcttggagtctcccgaggacacaaacaggcagagagggacgtgtagggagagttctttcctgttttctgtgctttcctttttacaggactcccggaaggccactcatggccatgccaggagctttctcagaaacagtcataaacgatctcttgagtctctttcttgtcctcccagctgagctttcttattccaccctttctggtgtctataggaatgcatgagagaccctggacgtttttctgctctcttctggccctccatggagccatgggcctcggcctcggcggctcctcaccctcacaatttatttcctcctcccgtgccagcccttcttttgtgtctgaaaccggttttaaaatgtgactctcccagagaagaagccgctggctgtatgaaacttgacggcgcttttgtaaggtgccacccccaaactttaaggtagctaaaccaatttttaaaagattcaatggcttgttcatcctccagatgtagctattgatgtacacttcgcaacggagtgttctgaaattgtggtggtcctgatttataggatttcataattaaaatgtctgctg

SEQ ID NO: 37 - DNA, 1221 bp, Homo sapiens
gaaacacacggctaagaagagagaggcaagggcaggagtgaaggagagagctgaagcctggggctccgagatggtcagaggatgggagacggggcagtgaaacaaggcttcttgtatcttcagcagcagcagacgtttggaaaggggcagaggaaggagctctcggggccagagggaaagcagagccggccctgcatggaggaaaatgaattgtacagcagcgcagtcacagtcggcccccacaaggaatttgctgtgaccatgagacctacagaagccagtgagaggtgccacctgcgggggtcctataccctccgggctggggagagtgccctggagctgtggggtgggcccgagccagggacccagctgtacgactggccctacaggtttctgcggcgctttgggcgggacaaggtaaccttttcctttgaggcaggccgtcgctgcgtctctggagagggcaactttgagttcgaaacccggcaaggcaatgagatcttcttggccctggaagaggccatctctgcccagaagaatgctgcacccgctacaccccaaccgcagccagccacaatccccgcgtcgctgccccggcctgatagcccctactctcggccgcatgactcactgccgccgccttcacccaccacaccggtgcctgctccacggcctcggggccaggagggggagtatgccgtgcccttcgatgcggtggcccgttccttggggaagaacttcaggggcatcttggcagtccctcctcagctcctggccgaccctctgtacgacagcattgaggagaccctgccccctcgacctgaccacatatacgatgagcccgagggagtggctgccctgtccctctatgacagcccgcaggagccccggggtgaggcatggaggaggcaggcgacagtgacagggaccctgctggcctccagcatgtccagccagccgggcaggatttctctgcttctggctggcagccaggaactgagtatgacaatgttgtactaaagaaaggcccaaagtgacagaggcagcagagggatggtccaccgccccttggcttctgctggtgactcctcctggccactgcatcagaagaacctcctctgccccttctggagcccgaggcctggcctgtcttcgttggggctgataaattgcctctcccagggcctgctgggtgagtcaccatcccaaagcaggaagggt

SEQ ID NO: 38 - DNA, 861 bp, Homo sapiens
ggcaggttgtgcagctggaggcagagcagtcctctctggggagcctgaagcaaacatggatcaagaaactgtaggcaatgttgtcctgttggccatcgtcaccctcatcagcgtggtccagaatggattctttgcccataaagtggagcacgaaagcaggaccccagaatggggaggagcttccagaggaccggaacacttgcctttgagcgggtctacactgccaaccagaactgtgtagatgcgtaccccactttcctcggctgtgctctggtctgcggggctactttgcagccaagttcctgctgcgtttgctggactgatgtacttgtttgtgaggcaaaagtactttgtcggttacctaggagagagaacgcagagcacccctggctacatatttgggaaacgcatcatactcttcctgttcctcatgtccgttgctgggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttttcacttatcctgttctctgaagatgttttgtgaccaggtttgtgttttcttaaaataaaatgcagagacatgttttaaaa

SEQ ID NO: 39 - DNA, 237 bp, Homo sapiens
gtctagggctgcttgaggtcaagttgacactgctccacgtgctgcacaagttccggttccaagcctgccctgagacccaggtaccgctgcagctagaatccaaatctgccctaggtccaaaaaatggtgtctatatcaagatcgtatcccgctgacacagaaggctgccgggtggggggagggcacccccaaattcaaagaaaaccctaagtgtggatgttcagaattttggaaaaa

SEQ ID NO: 40 - DNA, 316 bp, Homo sapiens
  misc_feature (99)..(120): n is defined as selected from the group consisting of a, c, g, and t
aaagaaaactgtgagagagagaatttttaaaaagcagctggggcctgaggtttctcccccagtaccctgggtcacctcagcccagagctggcggcangnccccagcccctcatgtcagancccccctgtgtactgtaacctggtggaccttcgccgctgtcctcggtccccacccccaggccctgcatgccccctgctgcagaggctggatgcctgggagcagcacctggaccccaactctggacgctgcttctacataaattcactgactggctgcaagtcctggaagcccccgcgccgcagtcgcagcgagacg

SEQ ID NO: 41 - DNA, 260 bp, Homo sapiens
ggacttttccttgtacatcattagccatgtaggcattatcatctccttggtgtgcctcgtcttggccatcgccacctttctgctgtgtcgctccatccgaaatcacaacacctacctccacctgcacctctgcgtgtgtctcctcttggcgaagactctcttcctcgccggtatacacaagactgacaacaagacgggctgcgccatcatcgcgggcttcctgcactaccttttccttgcctgcttcttctggatgctgg

SEQ ID NO: 42 - DNA, 1397 bp, Homo sapiens
ggaggagcctctgccagactggagagaagcaggcctgagcctccccaaaggcagctcctggggactcccaggaccacaggctgagacgagacgcagggtggctggaggaagtgagaggtgaactcagcctgggactggctgggcgagactctccacctgctccctgggaccatcgcccaccatgggctgtggcccagcagctgcgggccgagagtgactttgaacagcttccggatgatgttgccatctcggccaacattgctgacatcgaggagaagagaggcttcaccagccactttgttttcgtcatcgaggtgaagacaaaaggaggatccaagtacctcatctaccgccgctaccgccagttccatgctttgcagagcaagctggaggagcgcttcgggccagacagcaagagcagtgccctggcctgtaccctgcccacactcccagccaaagtctacgtgggtgtgaaacaggagatcgccgagatgcggatacctgccctcaacgcctacatgaagagcctgctcagcctgccggtctgggtgctgatggatgaggacgtccggatcttcttttaccagtcgccctatgactcagagcaggtgccccaggcactccgccggctccgcccgcgcacccggaaagtcaagagcgtgtccccacagggcaacagcgttgaccgcatggcagctccgagagcagaggctctatttgacttcactggaaacagcaaactggagctgaatttcaaagctggagatgtgatcttcctcctcagtcggatcaacaaagactggctggagggcactgtccggggagccacgggcatcttccctctctccttcgtgaagatcctcaaagacttccctgaggaggacgaccccaccaactggctgcgttgctactactacgaagacaccatcagcaccatcaaggacatcgcggtggaggaagatctcagcagcactcccctattgaaagacctgctggagctcacaaggcgggagttccagagagaggacatagctctgaattaccgggacgctgagggggatctggttcggctgctgtcggatgaggacgtagcgctcatggtgcggcaggctcgtggcctcccctcccagaagcgcctcttcccctggaagctgcacatcacgcagaaggacaactacagggtctacaacacgatgccatgagctgacggtgtccctggagcagtgaggggacaccagcaaaaaccttcagctctcagaggagattgggaccaggaaaacctgggaggatgggcagacttcctgtctttgaggctaatggacccgtggggcttgtaatctgtctctttctactatttacatctgatttaaataaaccattccatctgaaaggggaaa

SEQ ID NO: 43 - DNA, 531 bp, Homo sapiens
cagtgtgtcaaggatgatgagctctacgaggaagtgcggctgacgctggaaggctgcagcatagacgccgacatcgacagtttcatccaggccaagagcacgggcacagagccccccgctccggtgccctaccagaactattacgatcgggaggtcaccccgctgaccagcagccctggcatacagccgtcctgcggcatgataaagaggttctctggactgctgcacggaagtcccaagaccacttcgttggcagcttctgctgcgtccacagagaccctgacccccacccccgagcggaatgagggtgtctacacagccatcgcagtgcaggagatacagggaaacccggcctcaccagcccaggagtaccgggcgctctacgattatacagcgcagaacccagatgagctggacctgtccgcgggagacatcctggaggtgatcctggaaggggaggatggctggtggactgtggagaggaacgggcagcgtggcttcgtccctgggttcctacctggagaagctttg

SEQ ID NO: 44 - DNA, 207 bp, Homo sapiens
ggctcctgagagtgtgatcgagcgctgtagctcagtccgcgtggggagccgcacagcacccctgacccccacctccagggagcagatcctggcaaagatccgggattggggctcaggctcagacacgctgcgctgcctggcactggccacccgggacgcgcccccaaggaaggaggacatggagctggacgactgcagcaagtttgt

SEQ ID NO: 45 - DNA, 141 bp, Homo sapiens
tgctcagaggtttgagaagatgatcggaggcctgtacctgggtgagctggtgcggctggtgctggctcacttggcccggtgtggggtcctctttggtggctgcacctcccctgccctgctgagccaaggcagcatcctcct

SEQ ID NO: 46 - DNA, 312 bp, Homo sapiens
atttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaattggtgtatgcagctgtgcccctggccgcctaggtcggc

SEQ ID NO: 47 - DNA, 228 bp, Homo sapiens
ccctgttactgtaagccataagatacctgtttagggaagaagtcactgtcctaaaaatcagaatgcttttcaaacccaagggagagtgatttttggatttccatgtcacttctctcaggaagggtggcacatcggaggcaactttccctgcctgccccatgtgctctctaggttccccagcgagggtcaaactcccagagagcctgggtgaggggtccgaacacgggg

SEQ ID NO: 48 - DNA, 223 bp, Homo sapiens
ggagaaggtggtggtgtgatgctgttctctacggggagcagggccacccctggggtcgctttggggcggctctgacagtgctgggggatgtgaatggggacaagctgacagacgtggtcatcggggccccaggagaggaggagaaccggggtgctgtctacctgtttcacggagtcttgggacccagcatcagcccctcccacagccagcggatcgcgggctc

SEQ ID NO: 49 - DNA, 1445 bp, Homo sapiens
gcgtggggaagtctgcagccgagagtccagagaggaggaagctcctgccggctgagcgggcctggaggaagtgagcagcggggctcctgcctcccggcctggtccccgaagaccccagaagaacccggaacttgcttccattcggaatccagggaccaccctttgcactcagctaggcctttgttttcctgcgtggaaagcggttgggcttgggaggcgatggagccggagttcttgtacgacctgctgcagctccccaagggggtggagcccccagcggaggaggagctctcaaaaggaggaaagaagaaatacctgccacccacttcccggaaggaccccaaatttgaagaactgcagaaggtgttgatggagtggatcaatgccactcttctccccgagcacattgtggtccgcagcctggaggaggacatgttcgacgggctcatcctacaccacctattccagaggctggcggcgctcaagctggaagcagaggacatcgccctgacagccacaagccagaagcacaagcttcacagtggtgctggaggccgtgaaccggagtctgcagctggaggagtggcaggccaagtggagcgtggagagcatcttcaacaaggacctgttgtctaccctgcacctccttgtggccctggccaagcgcttccagcccgacctctccctcccaaccaacgttcaggtggaggtcatcactatgaggtaagcaccaaaagtggtctgaagtcagagaagttggtggaacagctcactgaatacagcacagacaaggacgagcctccaaaggacgtctttgatgaattatttaagctggctccggagaaagtgaacgcagtgcaagaggccatcgtgaactttgtcaaccagaagctggaccgcctgggcctgtctgtgcagaatctggacacccagtttgcagatggggtcatcttactcttgctgattggacaacttgaaggcttcttcctgcacttaaaggaattctacctcactcccaactctcctgcagaaatgctgcacaacgtcaccctggcgctggagctgctgaaggacgagggcctgctcagctgccctgtcagccctgaagatatcgtgaacaaggatgccaagagcacactgagggtgctctatggtctgttctgcaagcacacgcagaaggcacacagggacaggacgccccatggagccccgaattgaccctcactgcctccaaagcccagagcctgcctgtcagcccagctggagggcccgaggctgcagggtgtcctcccacagtcccgctgtttcctgtgcattcgtgacccgcttccctcccaccctgtctcctgtctccatcgttggattatctttgaacccccttgtgtggatcattttgagccgcctggccttgctcagtttattttaataaaagtatttctgggagg

SEQ ID NO: 50 - DNA, 334 bp, Homo sapiens
ccaccacgtccctggtcccagctcgggagcacatcagaggcttagaggcgagtgggaagggactcagacagtgcaggacgagaaacgcccgcggcaccaaagcccctcagagcgtcgcccccgcctctagttctagaaagtcagtttcccggcactggcaccccggaacctcaggggctgccgagctgggggggcgctcaagctgcgaggatccgggctgcccgcgagacgaggagcgggcgcccaggatggggtgcatgaagtccaagttcctccaggtcggaggcaatacattctcaaaaatgaaaccagcgccacccacatgtcctgtgta

SEQ ID NO: 51 - DNA, 236 bp, Homo sapiens
agtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatggg

SEQ ID NO: 52 - DNA, 495 bp, Homo sapiens
cttcgggcacctcaggaaggcaccttcctctgtcagaatggccaccatggtaccatccgtgttgtggcccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacaggctcccggagcgtgtggagctggcacccctgcctccttggcagccggtgggccagaattcaccctgcgctgccaagtggaggatgggtcgccccggaccagcctcacggtggtg

SEQ ID NO: 53 - DNA, 297 bp, Homo sapiens
gaccctcaccactccatggtgaggaaggccatggccaggggaaactgagtttcatccaatgtggagaggagcgttgtcctagagcagggcaactcccaaactgtgacctctgatcatcgtcccttccagcttgctggagtgtccagagagacagatttgccacaagctaggcttacttatatgctccaccctacagaaatgggaccccaagtacccaatcttccctttaggagaggcaggcaggtgggtgagcagcagatgtagtttccatttccctgggggtttaattttccaaac

SEQ ID NO: 54 - DNA, 239 bp, Homo sapiens
ctggcggctaccacggacaagcggacatccttctacctgcccctagatgccatcaagcagctgcacatcagcagcaccaccaccgtcagtgaggtcatccaggggctgctcaagaagttcatggttgtggacaatccccagaagtttgcactttttaagcggatacacaaggacggacaagtgctcttccagaaactctccattgctgaccgccccctctacctgcgcctgcttgctgg

SEQ ID NO: 55 - DNA, 376 bp, Homo sapiens
gcgatcttcccagcacagacgtttggacagagcaggctcctaaggtctccagaatgcccgtgccagcctcctggccccaccttcctagtcctttcctgctgatgacgctactgctggggggactcacaggggtagctggcgaggaagagctgcaggtgattcagcctgacaagtccatatcagttgcagctggagagtcggccactctgcactgcactgtgacttccctgatccctgtgggcccatcatgtggtttagaggagctggagcaggccgggaattaatctacaatcaaaaagaggccacttccccagggtaacaacagtttcagacctcacaaagagaaacaacatggacttttccatccgcatcagta

SEQ ID NO: 56 - DNA, 238 bp, Homo sapiens
gcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggca

SEQ ID NO: 57 - DNA, 181 bp, Homo sapiens
ggactatctcacaggtgatctggactggagcagcagcagtgtgagcgactctgacgagcggggcagcatgcagagcctcggcagtgatgagggctattccagcaccagcatcaagagaataaagctgcaggacagtcacaaggcgtgtcttggtctctaagagagtgggcactgcggctgt

SEQ ID NO: 58 - DNA, 1310 bp, Homo sapiens
ggcaaaattggagctgtccaggccaggacaccccgatgtccaggggcttccatacgaacaggcacatgggctgggaagtacatgaggcccctgggtagaagggtctgtcagttctctcctcccttgccctctgggagggtcctcctaacatagcttccaggaggtgggaggagcagttactgtcagcaggtgtcagccaggtgtcagcttctcctggggatctctagatgtctgcttgtgatttttggcaagtatatgcaaatgagcctcctctcctgccctgagacaagtatctgcagtgtgaacctggcagcctcagacccaaggggctcagaggaaacttctctggtttctagagctctgtgctccttcagagaagtcttccttccttccagtcagtgtccctgtgaagctgggatactcatttcctgtgtaccgggcaaacaccggattgctgattttgagaaatgcctctcgatggacctgtaacctgctggagtctgggatggtagctgtgggctggacttggctgatgggatgacccggtggctagtgcagcatcacacaagcctggttcaagtcttggctgtgtcatttcctgctgagggaccaggcactgaatttcctacctcttagggtcattacctatgaggttaaagctacctcatgggattgttatacgccactaatgttgaggcagacacctcttggcagggtgactgctcatcttagaccctccccttttctgcgaatttgggccccttgatcctctgatgggagctgaaaggatgagaggtgggcatctagatttagggaggctgttcaggctttgcaggtcccttacctgaacacatagaaaccctggagctgtggactgtgtccatgtgtgtgtgtttgtctgtgtgtgttgcgggggatgggcacctgcatgaatgtggtagagaaaatggctctgctcagagggaagatacgcatagcaaggcagggaccagaggaatcacaggcgcctggagagcagccgggcaccgcctccagggacctgccggcttccctcagtcctccaggggcccagcactcttcctttaggccctgtgagcgtcccttgtcaggatacattctctcattttgctgaagctgatttgattgggtgtctgtttctcgcagccaaaagagctctgaatgaggaaagtgcttctgtgctaactccccgcgtctcctgaatttcagtcattcatgtacccgcctcgaaatttttgcaatatctgtgtaccaactgtccatttacttaataaagaagttttctttaaatt

SEQ ID NO: 59 - DNA, 236 bp, Homo sapiens
atgtcaccttcctttcatgccaatttgtttccgtcatgagaatggactacatggtatacttcagcttcctcacctggattttcatccccctggttgtcatgtgcgccatctatcttgacatcttttacatcattcggaacaaactcagtctgaacttatctaactccaaagagacaggtgcattttatggacgggagttcaagacggctaagtccttgtttctggttcttttcttg

SEQ ID NO: 60 - DNA, 275 bp, Homo sapiens
gcaaaaaggagaagtatctatttgtgcaaagagtcacacagttgacagagtggaggccagtcccgagagaggctttgcagttcccacctcgggaagctccggcagaacccaggcgagggacagctccggacaggtgtggggtgcacactgaaaatgccacggtccttcctggtgaagagcaagaaggctcacacctaccaccagccccgtgtgcaggaagatgaaccgctctggccttctgcccttaccccggtgcccagagaccagggttcaag

SEQ ID NO: 61 - DNA, 408 bp, Homo sapiens
cggcacagtagagagcttccagggctggctggcgtgggatacccgtaccacagaaatgcagggaccattgcttcttccaggcctctgctttctgctgagcctctttggagctgtgactcagaaaaccaaaacttcctgtgctaagtgccccccaaatgcttcctgtgtcaataacactcactgcacctgcaaccatggatatacttctggatctgggcagaaactattcacattccccttggagacatgtaacgacattaatgaatgtacaccaccctatagtgtatattgtggatttaacgctgtgtgttacaatgtcgaaggaagtttctactgtcaatgtgtcccaggatatagactgcattctgggaatgaacaattcagtaattccaatgagaacacctgtca

SEQ ID NO: 62 - DNA, 916 bp, Homo sapiens
gtgcccgacagtgctggctcaggcaacgtcactcgctgctttgagcattacgagaagggcagcgtgccagtcctcatcatccacatcttcatcgtgttcagcttcttcctggtcttcctcatcatcctcttctgcaacctggtcatcatccgtaccttgctcatgcagccggtgcagcagcagcgcaacgctgaagtcaagcgccgggcgctgtggatggtgtgcacggtcttggcggtgttcatcatctgcttcgtgccccaccacgtggtgcagctgccctggacccttgctgagctgggcttccaggacagcaaattccaccaggccattaatgatgcacatcaggtcaccctctgcctccttagcaccaactgtgtcttagaccctgttatctactgtttcctcaccaagaagttccgcaagcacctcaccgaaaagttctacagcatgcgcagtagccggaaatgctcccgggccaccacggatacggtcactgaagtggttgtgccattcaaccagatccctggcaattccctcaaaaattagtccctgcttccaggcctgaagtcttctcctccatgaacatcatggactgagctgggggaagaagggatatctactgtggtctgggcaccacctctgtgggcactggtgggccattagatttggaggctacctcacctgggcagggatgatgggcagagccagggctgttggaaaatccagaactcaaatgagccccttcatccgcctgtgggcgcatactacagtaactgtgactgatgactttatcctgagtcccttaatcttatggggccggaaggaatgtcagggccaggtgcagaccttgggggaagactttaaaccacctagttctccccgatggggcatcggtctaaagctttgggggagt

SEQ ID NO: 63 - DNA, 248 bp, Homo sapiens
ctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagc

SEQ ID NO: 64 - DNA, 2253 bp, Homo sapiens
ttttcctgagtgcctatgatgacctaagtccccttctgggacctaaacccccaatctggaagggttcagggagtctggagggagaggcagcaggatgtggaaggcaggctctgggacagggtggggaagagcaggcatgctgggaagttggggaggacaagcaggctgagcctggaggcaggctagacatcagggaagaggcagagggaagtccagagaccaaggtggaggctggaaaggccagtgaggatagaggggaggctgggggaagccaagagacaaaagtcagattgagagaagggagtagggaagagacagaggccaaggaagagaagtccaaaggtcagaagaaggctgacagtatggaggctaaaggtgtggaggaaccaggaggagatgagtatacagatgagaaggaaaaagaaattgagagagaagaggatgaacaaagagaggaagcccaggtagaagctggaagggacctagagcaaggggcccaggaagatcaagttgctgaggagaaatgggaagttgtacagaaacaagaggctgagggagtcagagaggatgaggacaaaggacagagggagaaggggtacctgaagcaagaaaagaccaaggagatggtgaagacagcagaagcccagaagcagcaactgaaggaggagcaggggaggtcagcaaggaacgggagagtggggatggagaggctgagggagaccagagggctggagggtactatttagaagaggacaccctctctgaaggttcaggtgtagcgtccctggaggttgactgtgccaaagagggcaatccgcactcttctgagatggaagaggtagccccacagccacctcagccagaggagatggagcctgaggggcagcccagtccagacggctgtctatgcccctgttctcttggcctgggtggcgtgggcatgcgtctagcttccactctggttcaggtccaacaggtccgctctgtgcctgtggtgccccccaagccacagtttgccaagatgcccagtgcaatgtgtagcaagattcatgtggcacctgcaaatccatgcccgaggcctggccggcttgatgggactcctggagaaagggcttgggggtcccgagcttctcgatcctcttggaggaatgggggtagtctttcctttgatgctgctgtggccctagcccgggaccgccaaaggactgaggctcaaggagttcggcgaaccagacctgtactgagggtggggattactgcctcatccccagaacctccccttgtagcatgatctctgcccattctcctcggccccttagctgcctggagctcccatctgaaggtgcagaagggtctggatcccggagtcgtcttagtctgccccccagagaaccccaggttcctgaccccctgttgtcctctcagcgcaggtcatatgcatttgaaacacaggctaaccctgggaaaggtgaaggactgtgattaggaccacagccctgggcaaaggggaccagcaagttgtcttgaatctcccagggttcctgactagctgtctcctctgcagcatgagcagctgtagtgcccaactctataggctttggccctccagcttctctctttgactgtgggaggcactgccttggttggtttacctgaacttgtctccgacacaaagcacttatctcttaggagattcccaagaaagtcaacaagatcttgttcccagggagtgggtcattggccaaagggaacataaggtaggcagaaaacttaaaagagtttgttaaagtgaagactggagaaattcctcccttcctctgagctgtgaatctctcttcatgaaagccaaaggtagagacagggaggacagggccaggttagggccttccacacacaaacacttctagagttgcccattcctgttatgttcttggaccctaagatacctcctgtcccttttaaatccagattaagagaaacgtccaggaagagctctttgaagccctcaatatttgttggagggactggactcctctccagctccccaccctctgcctccagtcaccatgtgcaagagaggtcctgtacagatctctctgggctctcctttctcctttggaataacttgttcctatttcaggaaagggaaatggtgtcactcaggccctgggactgcttctccagccaggctggggccacaggtcccactctagtgaaggtcaatgtctcagaataaaagctgtatttttacac

SEQ ID NO: 65 - DNA, 850 bp, Homo sapiens
  misc_feature (291)..(291): n is defined as selected from the group consisting of a, c, g, and t
ccgaggggccggcttggcctgggatcgcctgtgccgtggcttggggtttagccctgctgctgaccataccctccttcctgtaccgggtggtccgggaggagtactttccaccaaaggtgttgtgtggcgtggactacagccacgacaaacggcgggagcgagccgtggccatcgtccggctggtcctgggcttcctgtggcctctactcacgctcacgatttgttacactttcatcctgctccggacgtggagccgcagggccacgcggtccaccaaagacactcaaaggnggtggtggcaatggtggccaagtttctttatcttctggttgccctaccaggtgacggggataatgatgtccttcctggagccatcgtcacccaccttcctgctgctgaataagctggactccctgtgtgtctcctttgcctacatcaactgctgcatcaaccccatcatctacgtggtggccggccagggcttccagggccgactgcggaaatccctccccagcctcctccggaacgtgttgactgaagagtccgtggttagggagagcaagtcattcacgcgctccacagtggacactatggcccagaagacccaggcagtgtaggcgacagcctcatgggccactgtggcccgatgtccccttccttcccggccattctccctcttgttttcacttcacttttcgtgggatggtgttaccttagctaactaactctcctccatgttgcctgtctttcccagacttgtccctccttttccagcgggactcttctcatccttcctcatttgcaaggtgaacacttccttctagggagcaccctcccaccccccacctgt

SEQ ID NO: 66 - DNA, 299 bp, Homo sapiens
ggagaattctccatcaagatgaaaggtctggctggctttgcacgcctctgtcctggagatcaatatgaaattttcatgaagtatggccggcagcggtggaaactgaaaggcaaaatagaagtaaatggcaagcagagctgggatggagaagaaacagtttttctgcccctgatagttgggttcatctccatcaaggtcacggagctcaaagggctagcaactcacatcctggtaggtagcgtgacctgtgagaccaaagagctgtttgcagcccgacctcaggtagtggctgtcgacat

SEQ ID NO: 67 - DNA, 321 bp, Homo sapiens
gtctgcagaactctactactcagtctctctggctgggaacatttgaggacattgatgacgaagatattagttcagccatgctcaagggactctgtgaaatgtctgttgagagcctcaacctgcaggaacaccgcttctctgacatctcatccaccacatttcagtgcttcacccaactccaagaattggatctgacagcaactcacttgaaagggttaccctctgggatgaagggtctgaacttgctcaagaaattagttctcagtgtaaatcatttcgatcaattgtgtcaaatcagtgctgccaatttcccctccctta

SEQ ID NO: 68 - DNA, 453 bp, Homo sapiens
cggaaacagagagagaacagaagaagagaaagctcagcaaattttcttgccatacttcatgacttcactgtggctaagtgtggggaccagacaggacttcgtggagacatccaggtgctgaagccttcagctactgtctcagttttttgaagtttagcaatggcgtctttctctgctgagaccaattcaactgacctactctcacagccatggaatgagcccccagtaattctctccatggtcattctcagccttacttttttactgggattgccaggcaatggggctggtgctgtgggtggctggcctgaagatgcagcggacagtgaacacaatttggttcctccacctcaccttggcggacctcctctgctgcctctccttgcccttctcgctggctcacttggctctccagggacagtggccctacggcaggttcctatgcaagctcat

SEQ ID NO: 69 - DNA, 389 bp, Homo sapiens
aagacgcgcatggccatgtacaagaagagcctccacatcaacggcgggggcagcgcagctgagcagcgtgagaagatcaagcagttctcccagcaggaggagaagaggcagaagtcggagcggctgcagcaacagcagaaacacgagaaccagatgcgggacatgctggcgcagtgtgagagcaacatgagcgagctgcagcagctgcagaatgaaaagtgccacctcctggtagagcacgaaacccagaaactgaaggccctggatgagagccataaccagaacctgaaggaatggcgggacaagcttcggccgcgcaagaaggctctggaagaggatctgaaccagaagaagcgggagcaggagatgttcttcaagctgagcgagga

SEQ ID NO: 70 - DNA, 872 bp, Homo sapiens
tgccaggccaggagtgggctggagctgctgaggcccagaaggcccgcatagttcctcaccaggagggtctccctccagcacaggtcccgcctgctcagtccctgtgctttccaagtggagggggtgtcagctcggtcaccaagagaagtgacttcccaggagcaccagtcccctcacagaggtgctgtgagcgaggcctccttcggtccaggcagaacctcaggcagtctctcctggtcctgcatgtgtgcaactccagctaataataggtattttatatacagatggacagatatttttttaaactggggagttttcctgatcttgggtcctctttagggggccagacaagaaaggcctacggtttctggagcaaacctgtcccccacctgtgaccagtcactctggggcaagcatcactgggaagggcatagccggagacctttctcctagtgactggtagtcaagtggggcattttaggatgtcagggccccagggtgacatgtgtccagcttttctatggcagcagaggcctttccctcagacaagctacaaagtgcccaggatgccatccaccatggtccccttcagtacctcacgcatggtccccagcttctcctaggatcccagtggcatttatcagtggccagctacctgccaggcccgggctggggcacggtctgcggccatgaggccaggccgcctcccgcacccctaccccgtggcagcagcatacctctgcatttctggaatgtgtgtgcatcaatgatgtttgtatatttgaggcatttaaaaatctattttcgttacgagggcaaatgaagaatgaatattgatcttaaaaaaagaaaagaagacaaaaaaaataccc

SEQ ID NO: 71 - DNA, 262 bp, Homo sapiens
cggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagtcagctgttca

SEQ ID NO: 72 - DNA, 20 bp, Homo sapiens
cagacggtgactttctgggt

SEQ ID NO: 73 - DNA, 20 bp, Homo sapiens
agagggagggtaaggctgag

SEQ ID NO: 74 - DNA, 20 bp, Homo sapiens
agggtcctgagaagagggag

SEQ ID NO: 75 - DNA, 20 bp, Homo sapiens
gaagagggagggtaaggctg

SEQ ID NO: 76 - DNA, 20 bp, Homo sapiens
aggatcgaggacagtgcaac

SEQ ID NO: 77 - DNA, 23 bp, Homo sapiens
ggcggccaggggcacagctgcat

SEQ ID NO: 78 - DNA, 22 bp, Homo sapiens
gcggccaggggcacagctgcat

SEQ ID NO: 79 - DNA, 22 bp, Homo sapiens
ccgacctaggcggccaggggca

(19) United States
(12) Patent Application Publication
(10) Pub. No.: US 2004/0002084 A1
(43) Pub. Date: Jan. 1, 2004

(54) Nucleic acids, polypeptides, vectors, and cells derived from activated eosinophil cells

(76) Inventors: Stanton B. Dotson, Chesterfield, MO (US); Xiao Jun Ma, San Diego, CA (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/350,923

(22) Filed: Jan. 24, 2003

Related U.S. Application Data
(63) Continuation of application No. 09/454,280, filed on Dec. 3, 1999, now abandoned.
(60) Provisional application No. 60/111,006, filed on Dec. 4, 1998.

(51) Int. Cl. C12Q 1/68; C07H 21/04; C12P 21/02; C12N 5/06; C07K 14/705; C12N 9/12; C12N 9/64

(52) U.S. Cl. 435/6; 435/69.1; 435/320.1; 435/325; 530/350; 536/23.5; 435/194; 435/226

(57) ABSTRACT

This invention relates to the immune response activity of eosinophils, and especially activated eosinophils, and provides isolated nucleic acids, polypeptides, including fragments, subunits and variants of the nucleic acids and polypeptides, as well as related compositions, probes, vectors, host cells, antibodies, and methods. In one aspect, the invention provides isolated nucleic acids encoding specific eosinophil-derived polypeptides or fragments or variants. Generally, the nucleic acids comprise a sequence encoding at least one domain or region associated with an eosinophil biochemical pathway function or associated with activated eosinophils. And, generally, corresponding domains or regions of polypeptides of the invention have at least a five amino acid fragment encoded by one of SEQ NOS: 1-71. Both the nucleic acids and the polypeptides of the invention can be used as probes, targets, or elements of numerous methods for identifying agents or compositions useful in the treatment of asthma or in methods for diagnosing or identifying eosinophil-related disorders in humans and mammals.

### PRIORITY

The present application is a continuation of U.S. application Ser. No. 09/454,280, filed Dec. 3, 1999, which claims priority under Title 35, United States Code, §119 of U.S. Provisional Application Serial No. 60/111,006, filed Dec. 4, 1998.

### FIELD OF THE INVENTION

This invention relates to nucleic acids derived from activated eosinophil cells as well as compounds and compositions made using these nucleic acids. A variety of nucleic acid and polypeptide compounds and compositions are described and specifically disclosed. The nucleic acids or polypeptides may be contained within vectors or host cells and then used to produce agents such as nucleic acids, polypeptides, fragments of polypeptides, and variants of each. These agents can be used in methods to produce or to identify biological compounds and compositions related to one or more immune system functions. Cells containing one or more nucleic acids or polypeptides of the invention can also be used as targets in high-throughput screening methods, particularly in screening for compounds designed to identify compositions affecting allergic disease states, such as asthma.

### BACKGROUND OF THE INVENTION

Eosinophils and the Cellular Immune Response

Eosinophils play an important role in the cellular immune system to provide an organism with the ability to attack infection and disease in any tissue or organ. Generally, the circulating cells of the immune system remain confined within the blood and lymph. Cells presented with an antigenic challenge, during infection or disease, must be able to traverse tissue and act directly at the site of the antigen. Eosinophils make up a specific subset of immune cells that respond by invading tissue and directly attacking infected cells.

In one aspect of the cellular immune response, secreted signals cascade from one cell type to another, activating the cells to perform specific immune functions. The ultimate position in the eosinophil cascade belongs to mast cells that, when triggered by antigen, release eosinophil growth and differentiation factors and eosinophil chemotactic factors (for example, interferons IL-3 and IL-5, and GM-CSF). As a result, eosinophils are activated and begin to congregate. Eosinophils then release IL-5 and other growth factors, chemotactic factors, and toxic granule proteins (for example, leukotrienes, eosinophil major basic protein, platelet-activating factor, eosinophil cationic protein, and eosinophil peroxidase) (Branco Ferreiraand Palmo Carlos, Cytokines and Asthma, Invest. Allergol. Clin. Immunol. 8:141-148, (1998)). Together, these secreted products allow eosinophils to bind to and eventually penetrate vascular or endothelial tissue. Now free to move through the tissue of an organ, eosinophils seek out and attempt to destroy infected, diseased, or damaged cells through cytotoxic actions.

In another aspect of the cellular immune response, eosinophils directly respond to infectious agents or antibody-coated antigens. Eosinophils can identify certain parasitic organisms and respond directly to them by releasing cytotoxic agents. As part of the late phase immune response to antigens, eosinophils also play an important role in seeking out antigen-bearing cells. Once the immune system has been primed by a challenge from an antigen, IgG and IgE antibody molecules specific for that antigen will be present. Whenever a cell containing the antigen resurfaces, the IgG and IgE antibodies will bind to the cell. Then, eosinophils can directly bind antibody-coated cells and exert their cytotoxic effects.

Disorders Involving Eosinophils

One function of eosinophils is to destroy and invade the host tissue to eliminate antigen-bearing cells. An improper balance of eosinophil action, however, can lead to significant tissue damage. Eosinophelia and hypereosinophilia are disease states pathologically-associated with increased numbers of eosinophils, which lead to increased numbers of activated eosinophils invading and destroying host tissue.

Atopic diseases such as asthma, allergic rhinitis, atopic dermatitis, anaphylaxis, and allergic bronchopulmonary aspergillosis involve both congenital and environmental origins, and symptoms arise from an apparent predisposition to hyper-reactions toward specific antigens. More common and less severe manifestations of eosinophilia may include eczema, psoriasis, and emphysema. Eosinophilia is also associated with leukemia, lymphomas, and particularly ovarian cancer, and certain connective tissue disorders, acquired or congenital immune disorders, and pneumonia. Eosinophilia may also arise in graft-vs.-host disease. An imbalance in eosinophil action can lead to consequences which vary from bothersome to life threatening. Asthma, for example, will be specifically discussed below. Applications of this invention, however, are not limited to its potential effects on the treatment of asthma.

Asthma: A Specific and Costly Eosinophil-Related Disorder

Asthma is a complex, chronic disease affecting a large population worldwide. Serafin provides a brief description of the disease and the underlying etiology (Serafin, Drugs used in the treatment of asthma, In Hardman, and Limbird, eds. Goodman and Gilman's, The Pharmacological Basis of Therapeutics, 9^{th} ed., pp. 659-682, (1995)). The illness accounts for 1-5% of all doctors' office visits (over 500,000 hospital visits per year) and is the leading cause of pediatric hospital admissions in the U.S. Over 5,000 people die of asthmatic attacks each year.

One model for the underlying pathophysiology of asthma is the presence of a low level, chronic inflammation that predisposes individuals to bronchial hyperactivity, bronchialspasm, tissue damage, mucus secretion, and allergenic hypersensitivity (Serafin, supra, 1995). The chronic inflammation can be divided into two processes: the activation of certain immune cells and the infiltration of pro-inflammatory cells into lung tissue. Infiltration of pro-inflammatory cells involves activation of the endothelium, cell recognition between circulating lymphocytes and the activated endothelium, attachment of circulating lymphocytes, and migration into the air passages.

The major types of lymphocytes observed in asthmatic lungs include Th₂ T-cells, eosinophils, and to a lesser extent, basophils and macrophage cells. Once activated and recruited into the lung, each of these cell types contributes to the chronic inflammatory state. Activated eosinophils secrete major basic protein, ECP, EDNT, LTC₄, cytokines such as IL-1 and IL-6, and granulocyte macrophage colony stimulating factor. Eosinophils also generate destructive free radicals, such as superoxide and nitric oxide. The Th₂ T-cells produce a variety of pro-inflammatory cytokines. Basophils release histamine, IL-4 cytokine and LTC₄. Macrophage cells release a variety of pro-inflammatory mediators including tumor necrosis factor alpha, LTB₄, prostaglandin D2, tissue destructive proteases, and free radicals such as superoxide and nitric oxide.

While the causes of asthma remain unknown, the present understanding indicates two major pathophysiological conditions: the narrowing of the bronchiolar airways and inflammation. Accordingly, modern asthma treatments can be divided into two classes, each class addressing one of these conditions (Serafin, supra, 1995). The first class of asthma drugs is bronchodialators, such as theophylline, anticholinergics, and B2-antagonists. They act to relax airway smooth muscles, thereby rapidly increasing the diameter of air passages. However, these drugs merely relieve the symptoms of an asthmatic attack rather than prevent the attack in the first place. Drugs that prevent asthma attacks would be preferable.

The second class is anti-inflammatory drugs, such as glucocorticoids and other immunomodulators. These drugs alleviate symptoms by suppressing the underlying inflammatory responses, which is what predisposes individuals to bronchorestriction. Although effective at reducing the number and severity of asthmatic attacks, the general immune suppressing effect of anti-inflammatory drugs limits their widespread use. An anti-inflammatory drug with a more selective effect remains an important goal in the treatment of asthma.

### SUMMARY OF THE INVENTION

Genetic and Bioinformatic Analyses

Genes involved in the biology of activated eosinophils are disclosed in this specification. These genes represent new drug targets for defining and controlling eosinophil action at the molecular level, such as blocking the participation of eosinophils in the underlying inflammation of asthma. The new drug targets can be used in molecular diagnostics and in the further definition, diagnosis, and treatment of immunological disorders. As therapeutic targets, these genes provide opportunities for intervention in the recognition, adhesion, migration and activation of eosinophils. These genes also provide intervention targets for the recognition, adhesion, migration, and activation of other lymphocytes that contribute to asthma or other immune disorders, as well as any cell that utilizes common molecular pathways.

The new drug targets are identified by bioinformatic analysis and data mining of expressed sequence tag (EST) databases derived from sequencing cDNAs from normal and diseased tissues, including libraries prepared from activated eosinophils. The data mining effort uses sequence comparison techniques (based on BLAST comparison of individual ESTs) to evaluate ESTs preferentially observed in the target libraries versus control and/or normal libraries. Selected ESTs derived from the same gene are then assembled to generate longer contiguous sequences (contigs) and evaluated as therapeutic intervention targets. The final, highly-selected set of sequences represent genes encoding multiple molecular targets for intervention in cellular or immunological disorders, and inflammatory diseases preferentially, asthma. This same set of sequences also represent molecular markers associated with cellular or immunological disorders, and with inflammatory diseases such as asthma. These markers also have a variety of diagnostic uses.

General Overview of Aspects of the Invention

This invention provides nucleic acids derived from activated eosinophil cells. Nucleic acid probes, proteins, and polypeptides made using these nucleic acids or derived from the sequence information of these nucleic acids are also disclosed. These nucleic acids may be inserted into vectors or host cells and then used to produce agents, such as nucleic acids, polypeptides, fragments of polypeptides, and variants of each. These agents can be used in assays or in methods to produce biological compounds and compositions related to one or more immune system functions. Cells or samples derived from cells containing one or more nucleic acids, proteins, or polypeptides of the invention can also be used as targets in high-throughput screening, and preferably in screens designed to identify compounds or compositions affecting allergic disease states, such as asthma. For example, the invention provides a method of identifying a biologically-active compound or composition comprising examining the interaction between a protein, polypeptide, or fragment of the invention and a compound or composition, and comparing this to a similar interaction of the protein, polypeptide, or fragment of the invention with a control.

The invention also provides a compound or composition that is detectable by such a method.

The nucleic acid probes of the invention may be derived from those disclosed, such as a fragment of 10 nucleotides or more or a sequence with 70% to 99% identity to a fragment of at least 10 nucleotides. Numerous methods for defining or identifying probes for nucleic acid or sequence based analysis exist. Such probes can be used in hybridization assays or techniques, in a variety of PCR-type methods, or in computer-based searches of databases containing biological information. Exemplary methods include a method of identifying a nucleic acid which comprises the hybridization of a probe of the invention with a sample containing nucleic acid and the detection of stable hybrid nucleic acid molecules. Also included are methods of identifying a nucleic acid comprising contacting a PCR probe of the invention with a sample containing nucleic acid and producing multiple copies of a nucleic acid that hybridizes, or is at least minimally complementary, to the PCR probe. The invention also provides a computer-readable medium having recorded thereon the sequence information of one or more of SEQ NO:1 through SEQ NO: 71, or complements thereof. In addition, the invention provides a method of identifying a nucleic acid comprising providing a computer-readable medium of the invention and comparing nucleotide sequence information using computerized means.

This invention also provides methods of making or using the nucleic acids, vectors and/or host cells, such as using them for the production of eosinophilic nucleic acids and/or proteins or polypeptides by recombinant, homologous recombinant, synthetic, gene activation, and/or purification techniques. The protein and polypeptide so produced are also provided, as well as transgenic animals or a cell taken from a transgenic animal having an introduced nucleotide comprising a nucleic acid of the invention.

This invention also provides a compound or composition comprising one or more polypeptides, which comprise: 1) at least one fragment, segment, or domain of at least 15-1,000 contiguous amino acids, with at least one portion encoded by one or more of SEQ NOS: 1-71; 2) at least one amino acid sequence selected from those encoding at least one of SEQ NOS: 1-71; or 3) at least one modification corresponding to fragments, segments, or domains within one of SEQ NOS:1-71.

The present invention also provides eosinophil-derived proteins or polypeptides as described herein, wherein the protein or polypeptide has at least one activity selected from the group consisting of GTP concentration effecting (e.g., a G protein coupled receptor), cell adhesion promoting or inhibiting, protease activity, cell cytotoxic activity, kinase activity, cell growth or differentiation effecting activity, wound healing activity, inflammatory activity, anti-inflammatory activity, chemotactic or chemokinetic activity, or immunomodulating activity. Thus, an eosinophil-derived protein or polypeptide of the invention can be screened for a corresponding activity according to known methods or as described herein. The proteins or polypeptides may also exhibit a combination of activities, such as both inflammatory and cell cytotoxic activities.

This invention also provides an antibody, polyclonal or monoclonal, that specifically binds at least one epitope found in or specific to an eosinophil-derived protein or polypeptide or a protein or polypeptide, of fragment or variant thereof, of this invention. Antibodies can be generated by recombinant, synthetic, or hybridoma technologies.

This invention also provides methods for identifying compounds that bind or interact with a nucleic acid, protein, or polypeptide or the invention. For example, nucleic acids of the invention can be used to measure or detect mRNA in a cell, tissue, or biological sample suspected of expressing genes also expressed in eosinophils or activated eosinophils.

Exemplary Uses of the Invention

The nucleic acids of the invention can be useful in making targets for small molecule drug development:

a) Nucleic acids represent genes, which can be cloned, over-expressed in a bacteria, yeast, insect, mammalian or other cells. The active protein can be used in high throughput screening for novel binding agents, stimulators, or inhibitors;

b) Nucleic acids represent intracellular markers which are correlated to a cellular state. These markers, individually or in combination, can be measured in response to compounds to screen for those compounds that suppress or activate the genes and thus alter the state of the cell in a desired manner;

c) Nucleic acids can be used to clone out a promoter, which in turn can be linked to a reporter gene, such as luciferase, and the recombinant reporter construct used to screen compounds that suppress or activate the gene(s);

d) Nucleic acids can be used to identify transcription factors that modulate gene expression—these transcription factors can be cloned, over-expressed in a bacteria, yeast, insect, mammalian or other cell and the active transcription factor can be used in high throughout screening for small molecule inhibitors or activators of gene expression.

These nucleic acids can be useful for the direct generation of therapeutic compounds or compositions:

a) nucleic acids represent genes which can be cloned, over-expressed in a bacteria, yeast, insect, mammalian or other cell, and the encoded protein can be used as a protein therapeutic;

b) nucleic acids represent genes which can be directly injected to elicit therapeutic antibodies;

c) nucleic acids represent genes which can be cloned, expressed in a bacteria, yeast, insect, mammalian or other cell, and the protein can be used to generate therapeutic antibodies;

d) nucleic acids can be used to generate antisense DNA molecules useful to suppress or modulate gene expression and provide a therapeutic benefit;

e) nucleic acids can be used to generate antisense oligonucleotides (ODNs) useful to suppress gene expression and provide a therapeutic benefit;

f) nucleic acids can be used to generate sense DNA or sense ODNs, which will act by co-suppression to provide therapeutic benefit;

g) nucleic acids or genes are useful as gene therapy for activating or suppressing themselves, other genes, or entire pathways of genes.

The nucleic acids and sequence information disclosed facilitate the cloning of other complete genes. These include genetic elements such as the entire coding region, the promoter controlling gene transcription, and the untranslated region, which may control RNA stability and translation and identify and clone the genomic clone containing exon and intron information:

a) nucleic acids specify oligonucleotide templates or probes to amplify a full length gene;

b) nucleic acids and their clones can be labeled in a manner such that they can be used to hybridize to a corresponding full length gene in order to detect and clone a full length gene;

c) nucleic acids have utility in other procedures, not limited to the two previous examples, to clone a full length gene.

The sequences are useful for generating diagnostic kits and methods:

a) nucleic acids can be used to monitor gene expression in a cell or tissue, which reports on the state of cell or a cell response to a drug or environment;

b) nucleic acids can be used in a number of diagnostic platforms, including but not limited to:

i) specifying oligonucleotides, which can be used in procedures to determine the level of gene expression inferring the cellular state or cell response;

ii) generating labeled DNA, which will hybridized to mRNA or cDNA prepared from a tissue to determine the level of gene expression, which infers the cellular state or cell response;

iii) specifying primer/probe sets for use in quantitative PCR technology such as TaqMan technology;

iv) applying nucleic acids or any of the specified oligonucleotides onto an array or microarray, by themselves or as a set or in combination with other genes, to determine the level of gene expression which can be used to infer a cellular state of cell response;

vi) directly injecting nucleic acids into animals to elicit diagnostic antibodies;

vii) producing diagnostic antibodies from nucleic acids and genes by cloning, or by expressing in a bacteria, yeast, insect, archaebacteria, mammalian or other host cell, and the expressed polypeptide used to generate antibodies useful for ELISAs, Westerns, and other antibody-based diagnostics.

c) nucleic acids can be used to evaluate the response to compounds in animal models to facilitate drug discovery;

d) nucleic acids can be used for diagnosing the presence of specific transcripts that correspond to those present in activated eosinophil to, for example, identify patients exhibiting eosinophil-related disorders or to diagnose the extent of disease in a patient;

e) nucleic acids can be used for measuring the response of a patient and their disease to a given drug treatment.

These detailed descriptions are presented for illustrative purposes only and are not intended as a restriction on the scope of the invention. Rather, they are merely some of the embodiments that one skilled in the art would understand from the entire contents of this disclosure. All parts are by weight and temperatures are in Degrees centigrade unless otherwise indicated.

The following is a list of abbreviations and the corresponding meanings as used interchangeably herein:

IMDM=Iscove's modified Dulbecco's media

mg=milligram(s)

ml or mL=milliliter(s)

ODNs=oligonucleotides

PCR=polymerase chain reaction

RP-HPLC=reverse phase high performance liquid chromatography

μg or ug=microgram(s)

μl or ul=microliter(s)

The following is a list definitions of various terms used herein:

The term “altered” means that expression differs from the expression response of cells or tissues not exhibiting the phenotype.

The term “amino acid(s)” means all naturally occurring L-amino acids.

The term “chromosome walking” means a process of extending a genetic map by successive hybridization steps.

The term “cluster” means that BLAST scores from pairwise sequence comparisons of the member clones are similar enough to be considered identical with experimental error.

The term “complete complementarity” means that every nucleotide of one molecule is complementary to a nucleotide of another molecule.

The term “degenerate” means that two nucleic acid molecules encode for the same amino acid sequences but comprise different nucleotide sequences.

The term “exogenous genetic material” means any genetic material, whether naturally occurring or otherwise, from any source that is capable of being inserted into any organism.

The term “expansion” means the differentiation and proliferation of cells.

The term “expressed sequence tags (ESTs) means randomly sequenced members of a cDNA or complementary DNA library.

The term “expression response” means the mutation affecting the level or pattern of the expression encoded in part or whole by one or more nucleic acid molecules.

The term “fragment” means a nucleic acid molecule whose sequence is shorter than the target or identified nucleic acid molecule and having the identical, the substantial complement, or the substantial homologue of at least 10 contiguous nucleotides of the target or identified nucleic acid molecule.

The term “fusion molecule” means a protein-encoding molecule or fragment that upon expression, produces a fusion protein.

The term “fusion protein” means a protein or fragment thereof that comprises one or more additional peptide regions not derived from that protein.

The term “marker nucleic acid” means a nucleic acid molecule that is utilized to determine an attribute or feature (e.g., presence or absence, location, correlation, etc.) of a molecule, cell, or tissue.

The term “mimetic” refers to a compound having similar functional and/or structural properties to another known compound or a particular fragment of that known compound.

The term “phenotype” means any of one or more characteristics of an organism, tissue, or cell.

The term “probe” means an agent that is utilized to determine an attribute or feature (e.g. presence or absence, location, correlation, etc.) of a molecule, cell, tissue, or organism.

The term “product score” refers to a formula which indicates the strength of a BLAST match using the fraction of overlap of two sequences and the percent identity. The formula is as follows:

$$Product Score = \frac{BLAST Score \times Present Identity}{5 \times minimum \{ length ( Seq 1 ) , length ( Seq 2 ) \}}$$

The term “protein fragment” means a peptide or polypeptide molecule whose amino acid sequence comprises a subset of the amino acid sequence of that protein.

The term “protein molecule/peptide molecule” means any molecule that comprises five or more amino acids.

The term “recombinant” means any agent (e.g., DNA, peptide, etc.), that is, or results from, however indirectly, human manipulation of a nucleic acid molecule.

The term “selectable or screenable marker genes” means genes who's expression can be detected by a probe as a means of identifying or selecting for transformed cells.

The term “singleton” means a single clone.

The term “specifically bind” means that the binding of an antibody or peptide is not competitively inhibited by the presence of non-related molecules.

The term “specifically hybridizing” means that two nucleic acid molecules are capable of forming an anti-parallel, double-stranded nucleic acid structure.

The term “substantial complement” means that a nucleic acid sequence shares at least 80% sequence identity with the complement.

The term “substantial fragment” means a fragment which comprises at least 100 nucleotides.

The term “substantial homologue” means that a nucleic acid molecule shares at least 80% sequence identity with another.

The term “substantially hybridizing” means that two nucleic acid molecules can form an anti-parallel, double-stranded nucleic acid structure under conditions (e.g. salt and temperature) that permit hybridization of sequences that exhibit 90% sequence identity or greater with each other and exhibit this identity for at least a contiguous 50 nucleotides of the nucleic acid molecules.

The term “substantially purified” means that one or more molecules that are or may be present in a naturally occurring preparation containing the target molecule will have been removed or reduced in concentration.

The term “tissue sample” means any sample that comprises more than one cell.

### DETAILED DESCRIPTION OF EXEMPLARY EMBODIMENTS

A. General Concepts and Definitions

General reference texts that provide descriptions of known techniques are discussed herein. These include Current Protocols in Molecular Biology (Ausubel, et al., eds., John Wiley & Sons, N.Y. (1989), and supplements through September 1998), Molecular Cloning, A Laboratory Manual (Sambrook et al., 2nd Ed., Cold Spring Harbor Press, Cold Spring Harbor, N.Y. (1989)), Cells, a Laboratory Manual (Spector et al, eds. Cold Spring Harbor, N.Y. (1998)), and Current Protocols in Immunology (Coligan, ed., John Wiley and Sons, Toronto (1994)), each of which are specifically incorporated by reference in their entirety.

As used herein, two nucleic acid molecules are said to be capable of specifically hybridizing to one another if the two molecules are capable of forming an anti-parallel, double-stranded nucleic acid structure. A nucleic acid is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions.

Conventional stringency conditions are described by Sambrook, et al., Molecular Cloning, A Laboratory Manual, 2nd Ed., Cold Spring Harbor Press, Cold Spring Harbor, N.Y. (1989), and by Haymes, et al. Nucleic Acid Hybridization, A Practical Approach, IRL Press, Washington, D.C. (1985), the entirety of both is herein incorporated by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Ausubel, et al., Current Protocols in Molecular Biology, John Wiley & Sons, N.Y. (1989) (see especially sections 6.3.1-6.3.6). [This reference and the supplements through November 1998 are specifically incorporated herein by reference and can be relied to make or use any embodiment of the invention.] For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Temperature and salt conditions may be varied independently.

An agent, be it a naturally-occurring molecule or otherwise, may be “substantially-purified,” if desired. As used herein, “substantially-purified” means that one or more molecules present in a naturally occurring preparation containing that molecule will have been removed or will be present at a lower concentration than that at which it would normally be found.

An agent may also be said to be “isolated” from another specific component with which it occurred. Some of the methods described later lead to degrees of purification appropriate to identify single bands in electrophoresis gels. However, this degree of purification is not required.

The agents of the present invention will preferably be “biologically active” with respect to either a structural or a catalytic attribute, which includes the capacity of a nucleic acid to hybridize to another nucleic acid molecule, or the ability of a protein to be bound by an antibody (or to compete with another molecule for such binding), among others. Catalytic attributes involve the capacity of the agent to mediate a chemical reaction or response.

The agents of the present invention may also be recombinant. The term “recombinant” means any agent (e.g., DNA, peptide, etc.), that is or results from, however indirectly, human manipulation of a nucleic acid molecule. The recombination may occur inside a cell or in a tube.

It is understood that the agents of the present invention may be labeled with reagents that facilitate detection (e.g., fluorescent labels, Prober et al., Science 238: 336-340 (1987), Albarella et al., EP 144914;, chemical labels, Sheldon et al., U.S. Pat. No. 4,582,789, Albarella et al., U.S. Pat. No. 4,563,417; and modified bases, Miyoshi et al., EP 119448) all of which are incorporated by reference in their entirety)).

A hybridization probe of the invention can be any nucleic acid capable of being labeled and forming a double-stranded structure with another nucleic acid over a region large enough for the double stranded structure to be detected. Various types of labels and detection methods have been described.

A PCR probe is a nucleic acid capable of initiating a polymerase activity while in a double-stranded structure with another nucleic acid. For example, Krzesicki, et al., Am. J. Respir. Cell Mol. Biol. 16:693-701 (1997), incorporated by reference in its entirety, discusses the preparation of PCR probes for use in identifying nucleic acids of eosinophils. Other methods for determining the structure of PCR probes and PCR techniques have been described.

A region or fragment in a molecule with “substantial identity” to a region of a different molecule can be represented by a ratio. When the individual units (e.g., nucleotides or amino acids) of the two molecules are schematically positioned to exhibit the highest number of units in the same position over a specific region, a percentage identity of the units identical over the total number of units in the region is determined. Numerous algorithmic and computerized means for determining a percentage identity are known in the art. These means may allow for gaps in the region being considered in order to produce the highest percentage identity. In a preferred embodiment, a 10 nucleotide in length nucleic acid region or fragment of the invention has a percentage identity of about 70% to about 99% with a nucleic acid sequence existing within one of SEQ NO.: 1-71 or a complement of SEQ NO.: 1-71.

Modifications can be naturally provided or deliberately engineered into the nucleic acids, proteins, and polypeptides of the invention to generate variants. For example, modifications in the peptide or DNA sequences can be made by those skilled in the art using known techniques, such as site-directed mutagenesis. Modifications of interest in the protein sequences may include the alteration, substitution, replacement, insertion or deletion of one or more selected amino acid residues. For example, one or more cysteine residues may be deleted or replaced with another amino acid to alter the conformation of the molecule. Additional cysteine residues can also be added as a substitute at sites to promote disulfide bonding and increase stability. Techniques for identifying the sites for alteration, substitution, replacement, insertion or deletion are well known to those skilled in the art. Techniques for making alterations, substitutions, replacements, insertions or deletions (see, e.g., U.S. Pat. No. 4,518,584) are also well known in the art. Preferably, any modification of a protein, polypeptide, or nucleic acid of the invention will retain at least one of the structural or functional attributes of the molecule.

A variety of computerized means for identifying sequences derived from the SEQ NO.: 1-71 exists. These include the five implementations of BLAST, three designed for nucleotide sequences queries (BLASTN, BLASTX, and TBLASTX) and two designed for protein sequence queries (BLASTP and TBLASTN), as well as FASTA and others (Coulson, Trends in Biotechnology 12:76-80 (1994); Birren et al., Genome Analysis 1:543-559 (1997)). Other programs which use either individual sequences or make models from related sequences to further identify sequences derived from SEQ NO 1-SEQ NO 71 exist. Model building and searching programs includes HMMer (Eddy), MEME (Bailey and Elkan, Ismb 3: 21-29 (1995)) and PSI-BLAST (Altschul et al., Nucleic Acids Res 25: 3389-3402 (1997)). Another set of programs which use predicted, related, or known protein structures to further identify sequences derived from SEQ NO 1-SEQ NO 71 exists. Structure-based searching programs includes ORF and PROSITE. Other programs which use individual sequences or related groups of sequences rely on pattern discovery to further identify sequences derived from SEQ NO:1-71 exist. Pattern recognition programs include Teiresias (Rigoutsos, I. and A. Floratos, Bioinformatics 1: (1998)). These programs can search any appropriate database, such as GenBank, dbEST, EMBL, SwissProt, PIR, and GENES. Furthermore, computerized means for designing modifications in protein structure are also known in the art (Dahiyat and Mayo, Science 278:82-87 (1997)).

The following protein or polypeptide embodiments of the invention can be identified through assays known in the art, including high throughput screening assays. The proteins or polypeptides possess a detectable activity in a functional assay and can be identified by that functional assay. For example, a kinase activity assay is discussed in U.S. Pat. No. 5,759,787, and the references therein (incorporated by reference in its entirety). Similar examples for each of the activities described exist and can be relied on to make or use aspects of this invention.

Kinase molecules of the invention facilitate the addition of a phosphate group onto another molecule, or, are structurally homologous to a protein that exhibits kinase activity. A number of kinase molecules are known in the art and some have been identified as being involved in biochemical pathways specific to immune cells.

G protein-coupled receptor molecules of the invention exhibit a GTP concentration effecting activity or a seven-member membrane spanning structure, or both. A number of G protein-coupled receptors have been described in the art and some have been identified as being involved in biochemical pathways specific to immune cells.

Protease molecules of the invention exhibit a peptide bond-hydrolyzing activity, or are structurally homologous to a protein that exhibits protease activity. A number of proteases are known in the art and some have been identified as being involved in biochemical pathways specific to immune cells.

Peroxidase molecules of the invention exhibit peroxide molecule concentration effecting activity or are structurally homologous to a protein exhibiting peroxidase activity. A number of peroxidase molecules are known in the art and some have been identified as being involved in biochemical pathways specific to immune cells. One example is eosinophil peroxidase.

Cell adhesion molecules of the invention exhibit a cell-to-cell interaction effecting activity or are structurally homologous to a protein exhibiting cell adhesion activity. A number of cell adhesion molecules are known in the art and some have been identified as being involved in specific functions of immune cells and vascular or endothelial cells. Integrins are one such example of cell adhesion molecules.

The cytotoxic activity of a molecule of the invention is the ability to destroy a cell or prevent its functioning in some manner. A number of proteins with cytotoxic activity have been identified, many of them associated with immune cells. Examples include eosinophil cationic protein and eosinophil major basic protein.

B. Identification of Initial ESTs

The initial ESTs identified from activated eosinophil libraries (Table 1) were analyzed for the presence of specific structural features. Tables 2-8 correlate that information. The EST sequences represent eosinophil-derived nucleic acids of the invention and can be used to create additional eosinophil-derived nucleic acids, proteins, and polypeptides of the invention.

Table 1. Nucleic acid molecules expressed in activated eosinophils with potential uses for the development of human therapeutics and diagnostics.

Table 2. Nucleic acid molecules from Table 1 representing signaling peptides, which are amenable to development of selective signaling peptide inhibitors which block signal transduction and suppress eosinophil activation.

Table 3. Nucleic acid molecules from Table 1 representing cell adhesion genes which are amenable to development of selective molecules which block cell adhesion, cell recognition and signal transduction and suppress eosinophil activation.

Table 4. Nucleic acid molecules from Table 1 representing G-protein coupled receptors, which are amenable to development of selective G-protein receptor ligand antagonists which block signal transduction, and suppress eosinophil activation.

Table 5. Nucleic acid molecules from Table 1 representing genes encoding secreted proteins which are amenable to development of protein therapeutic agents which block cell recognition, adhesion, migration, signal transduction and otherwise suppress eosinophil activation.

Table 6. Nucleic acid molecules from Table 1 representing cell surface molecules, including cell surface receptors, which are especially amenable to the development of selective ligands acting as pharmacological agonists and pharmacological antagonists which block cell recognition, adhesion, migration, signal transduction and otherwise suppress eosinophil activation.

Table 7. Nucleic acid molecules from Table 1 representing gene homologs or fragments thereof which exhibit a product score of 100.

Table 8. Nucleic acid molecules from Table 1 representing gene homologs or fragments thereof which exhibit a product score of 49-99.

Table 9. Nucleic acid molecules from Table 1 representing gene homologs or fragments thereof which exhibit a product score of 0.

Table 10. Nucleic acid molecules from Table 1 representing gene homologs or fragments thereof which exhibit a product score of 1-49.

C. Agents of the Invention

(a) Nucleic Acids

Agents of the present invention include nucleic acids and, more specifically, eosinophil-derived nucleic acids. A subset of the nucleic acid molecules of the invention includes nucleic acids that are associated with a gene or fragment thereof. Another subset of the nucleic acids of the invention includes those that encode proteins, polypeptides, or fragments of proteins or polypeptides. In a preferred embodiment, the nucleic acids of the invention are derived from the one or more EST sequences identified in Tables 1-8.

Fragment nucleic acids may encompass significant portion(s) of, or indeed most of, these nucleic acids. For example, a fragment nucleic acid can encompass an eosinophil gene homolog or fragment thereof. Alternatively, the fragments may comprise smaller oligonucleotides (having from about 10 to about 250 nucleotides, and more preferably, about 15 to about 30 nucleotide).

Nucleic acids or fragments thereof of the invention are capable of specifically hybridizing to other nucleic acids under certain circumstances. In a preferred embodiment, a nucleic acid of the present invention will specifically hybridize to one or more of the nucleic acids set forth in SEQ NO: 1 through SEQ NO: 71, or complements thereof, under moderately stringent conditions, for example at about 2.0×SSC and about 65° C.

In a particularly preferred embodiment, a nucleic acid of the invention will include those nucleic acids that specifically hybridize to one or more of the nucleic acids set forth in SEQ NO:1 through SEQ NO: 71, or complements thereof, under high stringency conditions. In one aspect of the present invention, the nucleic acid molecules of the present invention comprise one or more of the nucleic acid sequences set forth in SEQ NO: 1 through to SEQ NO: 71, or complements thereof.

In another aspect of the invention, one or more of the nucleic acid molecules of the present invention share between 100% and 90% sequence identity with one or more of the nucleic acid sequences set forth in SEQ NO: 1 through to SEQ NO: 71 or complements thereof. In a further aspect of the invention, one or more of the nucleic acids of the invention share between 100% and 95% sequence identity with one or more of the nucleic acid sequences set forth in SEQ NO: 1 through SEQ NO: 71, or complements thereof. In a more preferred aspect of the invention, one or more of the nucleic acids of the invention share between 100% and 98% sequence identity with one or more of the nucleic acid sequences set forth in SEQ NO: 1 through SEQ NO: 71, or complements thereof. In an even more preferred aspect of the invention, one or more of the nucleic acids of the invention share between 100% and 99% sequence identity with one or more of the sequences set forth in SEQ NO: 1 through SEQ NO: 71, or complements thereof.

(i) Nucleic Acids Comprising Genes or Fragments Thereof

This invention also provides genes corresponding to the cDNA sequences disclosed herein, also called eosinophil-derived nucleic acids. The corresponding genes can be isolated in accordance with known methods using the sequence information disclosed herein. The methods include the preparation of probes or primers from the disclosed sequence information for identification and/or amplification of genes in appropriate genomic libraries or other sources of genomic materials.

The invention provides naturally existing gene homologues or fragments thereof. Genomic sequences can be screened for the presence of protein homologues utilizing one or a number of different search algorithms have that been developed, such as the suite of BLAST programs. The BLASTX program allows the comparison of nucleic acid sequences in this invention to protein databases.

In a preferred embodiment of the present invention, the homologue protein or fragment thereof exhibits a BLASTX probability score of less than 1E-30, preferably a BLASTX probability score of between about 1E-30 and about 1E-12, even more preferably a BLASTX probability score of greater than 1E-12 with a nucleic acid or gene of this invention. In another preferred embodiment of the present invention, the nucleic acid molecule encoding the gene homologue or fragment thereof exhibits a % identity with its homologue of between about 25% and about 40%, more preferably of between about 40% and about 70%, even more preferably of between about 70% and about 90%, and even more preferably between about 90% and 99%. In another preferred embodiment, the gene homologue or fragment has a single nucleotide difference from its homologue.

As used herein, the term “product score” refers to a formula which indicates the strength of a BLAST match using the fraction of overlap of two sequences and the percent identity. This score is a normalized value between 0 and 100, with 100 indicating 100% identity over the entire length of the shorter of the two sequences, and 0 representing no shared identity between the sequences. Preferably, the homologue protein or fragment thereof exhibits a product score of 100. More preferably, the product score is between about 49 and about 99. Even more preferably, the protein or fragment exhibits a product score of 0. Most preferably, the homolog or fragment exhibits a product score between about 1 and about 49.

In another preferred embodiment, nucleic acid molecules having SEQ NO: 1 through SEQ NO: 71, or complements and fragments of either, can be utilized to obtain homologues equivalent to the naturally existing homologues. The degeneracy of the genetic code, which allows different nucleic acid sequences to code for the same protein or peptide, is known in the literature (see U.S. Pat. No. 4,757,006, the entirety of which is herein incorporated by reference). As used herein a nucleic acid molecule is degenerate of another nucleic acid molecule when the nucleic acid molecules encode for the same amino acid sequences but comprise different nucleotide sequences. An aspect of the present invention is that the nucleic acid molecules of the present invention include nucleic acid molecules that are degenerate of those set forth in SEQ NO: 1 through to SEQ NO: 71 or complements thereof.

In a further aspect of the present invention, one or more of the nucleic acid molecules of the present invention differ in nucleic acid sequence from those encoding a homologue or fragment thereof in SEQ NO: 1 through SEQ NO: 71, or complements thereof, due to the degeneracy in the genetic code in that they encode the same protein but differ in nucleic acid sequence. In another further aspect of the present invention, one or more of the nucleic acid molecules of the present invention differ in nucleic acid sequence from those encoding an homologue of fragment thereof in SEQ NO: 1 through SEQ NO: 71, or complements thereof, due to fact that the different nucleic acid sequence encodes a protein having one or more conservative amino acid residue. Examples of conservative substitutions are set forth below. Codons capable of coding for such conservative substitutions are well known in the art.

| | |
|---|---|
| Original Residue | Conservative Substitutions |

| | |
|---|---|
| Ala | ser |
| Arg | lys |
| Asn | gln; his |
| Asp | glu |
| Cys | ser; ala |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

(ii) Nucleic Acids Comprising Regulatory Elements

One class of agents of the invention includes nucleic acids having promoter regions or partial promoter regions or regulatory elements. Promoter regions are typically found upstream of the trinuclotide ATG sequence at the start site of a protein coding region. The term “promoter region” is a region of a nucleic acid that is capable, when located in cis to a nucleic acid sequence that encodes for a protein or peptide, of functioning in a way that directs expression of one or more mRNA molecules.

The nucleic acids of the invention may be used to isolate promoters of cell-enhanced, cell-specific, tissue-enhanced, tissue-specific, developmentally- or physiologically-regulated expression profiles. Isolation and functional analysis of the 5′ flanking promoter sequences from genomic libraries, for example, using genomic screening methods and PCR techniques, results in the isolation of useful promoters and transcriptional regulatory elements. These methods are known to those of skill in the art and have been described (see, for example, Birren et al., Genome Analysis: Analyzing DNA, 1, (1997), Cold Spring Harbor Laboratory Press, Cold Spring Harbor, N.Y., the entirety of which is incorporated by reference).

For example, in one embodiment, a regulatory element is detected by incubating nucleic acid(s), or preferably fragments such as ESTs, with members of genomic libraries (e.g., of hematopoietic, lymphpopoietic, or eosinophil cell line origin) and recovering clones that hybridize to the nucleic acid(s). Sequencing techniques can then identify regulatory elements from known sequence motifs or known assays for detecting regulatory sequences within a certain proximity to transcription and translation start and stop sites can be used. In a second embodiment, methods of “chromosome walking,” or inverse PCR may be used to obtain regulatory elements (Frohman et al., Proc. Natl. Acad. Sci. (U.S.A.) 85:8998-9002 (1988); Ohara et al., Proc. Natl. Acad. Sci. (U.S.A.) 86: 5673-5677 (1989); Pang et al., Biotechniques 22(6): 1046-1048 (1977); Huang et al., Methods Mol. Biol. 69: 89-96 (1997); Huang, et al., Method Mol. Biol. 67:287-294 (1997); Benkel et al., Genet. Anal. 13: 123-127 (1996); Hartl et al., Methods Mol. Biol. 58: 293-301 (1996), all of which are incorporated by reference in their entirety).

Promoters and regulatory elements obtained utilizing the nucleic acids of the invention can also be modified to affect their control characteristics. Examples of these modifications include, but are not limited to, enhancer sequences as reported by Kay et al., Science 236:1299 (1987), incorporated by reference in its entirety. Genetic elements such as these can be used to enhance gene expression of new and existing proteins or polypeptides.

(b) Proteins and Polypeptides

A class of agents of the present invention also comprises one or more of the protein or peptide molecules or complements thereof encoded by a nucleic acid molecule comprising a gene, or fragments or complements of the nucleic acid molecules located within SEQ NO: 1 through SEQ NO: 71. Protein and peptide molecules can be identified using known protein or peptide molecules as a target sequence or target motif in the BLAST programs of the present invention.

As used herein, the term “protein” or “polypeptide” includes any molecule that comprises five or more amino acids. Proteins or peptides may undergo a variety of modifications, including post-translational modifications, such as disulfide bond formation, glycosylation, phosphorylation, or oligomerization. The term “protein” or “polypeptide” includes any protein molecule that is modified by any biological or non-biological process. The terms “amino acid” and “amino acids” refer to all naturally occurring L-amino acids. This definition is meant to include norleucine, ornithine, homocysteine, and homoserine.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). A fusion protein or peptide molecule of the present invention is preferably produced via recombinant means.

Another class of agents comprises protein or peptide molecules encoded by SEQ NO: 1 through SEQ NO: 71 or complements thereof or, fragments or fusions thereof in which conservative, non-essential, or not relevant, amino acid residues have been added, replaced, or deleted. An example is the homologue protein of an eosinophil-derived protein. Such a homologue can be obtained by any of a variety of methods. For example, as indicated above, one or more of the disclosed sequences (SEQ NO: 1 through SEQ NO: 71, or complements thereof) will be used to define a pair of primers that may be used to isolate the homologue-encoding nucleic acid molecules from any desired species. Such molecules can be expressed to yield homologs by recombinant means.

Proteins or polypeptides of the invention can be expressed as variants that facilitate purification. For example, a fusion protein to such proteins as maltose binding protein (MBP), glutathione-S-transferase (GST) or thioredoxin (TRX) are known in the art [New England BioLab, Beverly, Mass., Pharmacia, Piscataway, N.J., and InVitrogen, San Diego, Calif.]. The polypeptide or protein can also be a tagged variant to facilitate purification, such as with histidine or methionine rich regions [His-Tag; available from LifeTechnologies Inc, Gaithersburg, Md.] that bind to metal ion affinity chromatography columns, or with an epitope that binds to a specific antibody [Flag, available from Kodak, New Haven, Conn.]. An exemplary, non-limiting list of commercially available vectors suitable for fusion protein expression includes: pBR322 (Promega); pGEX (Amersham); pT7 (USB); pET (Novagen); pIBI (IBI); pProEX-1 (Gibco/BRL); pBluescript II (Stratagene); pTZ18R and pTZ19R (USB); pSE420 (Invitrogen); pAc360 (Invitrogen); pBlueBac (Invitrogen); pBAcPAK (Clontech); pHIL (Invitrogen); pYES2 (Invitrogen); pcDNA (Invitrogen); and pREP (Invitrogen).

A number of other purification methods or means are also known and can be used. Reverse-phase high performance liquid chromatography (RP-HPLC), optionally employing hydrophobic RP-HPLC media, e.g., silica gel, further purify the protein. Combinations of methods and means can also be employed to provide a substantially purified recombinant polypeptide or protein.

The polypeptide or protein of the invention may also be expressed via transgenic animals. Methods and means employing the milk of transgenic domestic animals are known in the art.

One or more of the proteins, polypeptides, or fragments may be produced via chemical synthesis. Methods for synthetic construction are known to those skilled in the art. The synthetically-constructed sequences, by virtue of sharing primary, secondary or tertiary structural and/or conformational characteristics with proteins, may possess biological properties in common, including protein activity. Thus, they may be employed as biologically active or immunological substitutes for natural, purified proteins in screening of therapeutic compounds and in immunological processes for the development of antibodies.

(c) Antibodies

One aspect of the present invention concerns antibodies, single-chain antigen binding molecules, or other proteins that specifically bind to one or more of the protein or peptide molecules of the present invention and their homologues, fusions or fragments. Such antibodies may be used to quantitatively or qualitatively detect the protein or peptide molecules of the present invention. As used herein, an antibody or peptide is said to “specifically bind” to a protein or peptide molecule of the present invention if such binding is not competitively inhibited by the presence of non-related molecules.

Nucleic acid molecules that encode all or part of the protein of the present invention can be expressed, by recombinant means, to yield protein or peptides that can in turn be used to elicit antibodies that are capable of binding the expressed protein or peptide. Such antibodies may be used in immunoassays for that protein. Such protein-encoding molecules or their fragments may be a “fusion” molecule (i.e., a part of a larger nucleic acid molecule) such that, upon expression, a fusion protein is produced. It is understood that any of the nucleic acid molecules of the present invention may be expressed, by recombinant means, to yield proteins or peptides encoded by these nucleic acid molecules.

The antibodies that specifically bind proteins and protein fragments of the present invention may be polyclonal or monoclonal, and may comprise intact immunoglobulins, or antigen binding portions of immunoglobulins (such as (F(ab′), F(ab′)₂ fragments), or single-chain immunoglobulins producible, for example, via recombinant means. Conditions and procedures for the construction, manipulation and isolation of antibodies (see, for example, Harlow and Lane, Antibodies: A Laboratory Manual, Cold Spring Harbor Press, Cold Spring Harbor, N.Y. (1988), the entirety of which is herein incorporated by reference) are well known in the art.

Murine monoclonal antibodies are particularly preferred. BALB/c mice are preferred for this purpose, however, equivalent strains may also be used. The animals are preferably immunized with approximately 25 μg of purified protein (or fragment thereof) that has been emulsified a suitable adjuvant, such as TiterMax adjuvant (Vaxcel, Norcross, Ga.). Immunization is preferably conducted at two intramuscular sites, one intraperitoneal site, and one subcutaneous site at the base of the tail. An additional i.v. injection of approximately 25 μg of antigen is preferably given in normal saline three weeks later. After approximately 11 days following the second injection, the mice may be bled and the blood screened for the presence of anti-protein or peptide antibodies. Preferably, a direct binding Enzyme-Linked Immunoassay (ELISA) is employed for this purpose.

More preferably, the mouse having the highest antibody titer is given a third i.v. injection of approximately 25 μg of the same protein or fragment. The splenic leukocytes from this animal may be recovered 3 days later, and are then permitted to fuse, most preferably, using polyethylene glycol, with cells of a suitable myeloma cell line (such as, for example, the P3X63Ag8.653 myeloma cell line). Hybridoma cells are selected by culturing the cells under “HAT” (hypoxanthine-aminopterin-thymine) selection for about one week. The resulting clones may then be screened for their capacity to produce monoclonal antibodies (“mAbs), preferably by direct ELISA.

In one embodiment, anti-protein or peptide monoclonal antibodies are isolated using a fusion of a protein, protein fragment, or peptide of the present invention, or conjugate of a protein, protein fragment, or peptide of the present invention, as immunogens. Thus, for example, a group of mice can be immunized using a fusion protein emulsified in Freund's complete adjuvant (e.g. approximately 50 μg of antigen per immunization). At three week intervals, an identical amount of antigen is emulsified in Freund's incomplete adjuvant and used to immunize the animals. Ten days following the third immunization, serum samples are taken and evaluated for the presence of antibody. If antibody titers are too low, a fourth booster can be employed. Polysera capable of binding the protein or peptide can also be obtained using this method.

In a preferred procedure for obtaining monoclonal antibodies, the spleens of the above-described immunized mice are removed, disrupted, and immune splenocytes are isolated over a ficoll gradient. The isolated splenocytes are fused, using polyethylene glycol with BALB/c-derived HGPRT (hypoxanthine guanine phosphoribosyl transferase) deficient P3x63xAg8.653 plasmacytoma cells. The fused cells are plated into 96-well microtiter plates and screened for hybridoma fusion cells by their capacity to grow in culture medium supplemented with hypothanthine, aminopterin and thymidine for approximately 2-3 weeks.

Hybridoma cells that arise from such incubation are preferably screened for their capacity to produce an immunoglobulin that binds to a protein of interest. An indirect ELISA may be used for this purpose. In brief, the supernatants of hybridomas are incubated in microtiter wells that contain immobilized protein. After washing, the titer of bound immunoglobulin can be determined using, for example, a goat anti-mouse antibody conjugated to horseradish peroxidase. After additional washing, the amount of immobilized enzyme is determined (for example through the use of a chromogenic substrate). Such screening is performed as quickly as possible after the identification of the hybridoma in order to ensure that a desired clone is not overgrown by non-secreting neighbors. Desirably, the fusion plates are screened several times since the rates of hybridoma growth vary. In a preferred sub-embodiment, a different antigenic form of immunogen may be used to screen the hybridoma. Thus, for example, the splenocytes may be immunized with one immunogen, but the resulting hybridomas can be screened using a different immunogen. It is understood that any of the protein or peptide molecules of the present invention may be used to raise antibodies.

As discussed below, such antibody molecules or their fragments may be used for diagnostic purposes. Where the antibodies are intended for diagnostic purposes, it may be desirable to derivatize them, for example with a ligand group (such as biotin) or a detectable marker group (such as a fluorescent group, a radioisotope or an enzyme).

The ability to produce antibodies that bind the protein or peptide molecules of the present invention permits the identification of mimetic compounds of those molecules. A “mimetic compound” is a synthesized compound, or a fragment of that compound with similar properties to a naturally-occurring compound or fragment of that compound which exhibits an ability to specifically bind to antibodies directed against that compound. Mimetic compounds can be synthesized chemically. Combinatorial chemistry techniques, for example, can be used to produce libraries of peptides (see WO 9700267), polyketides (see WO 960968), peptide analogues (see WO 9635781, WO 9635122, and WO 9640732), oligonucleotides for use as mimetic compounds derived from this invention. Mimetic compounds and libraries can also be generated through recombinant DNA-derived techniques. For example, phage display libraries (see WO 9709436), DNA shuffling (see U.S. Pat. No. 5,811,238) other directed or random mutagenesis techniques can produce libraries of expressed mimetic compounds. It is understood that any of the agents of the present invention can be substantially purified and/or be biologically active and/or recombinant.

(d) Mammalian Constructs and Transformed Mammalian Cells

The present invention also relates to methods for obtaining a recombinant mammalian host cell, comprising introducing exogenous genetic material into a mammalian host cell. The present invention also relates to an insect cell comprising a mammalian cell containing a mammalian recombinant vector. The present invention also relates to methods for obtaining a recombinant mammalian host cell, comprising introducing into a mammalian cell exogenous genetic material.

Mammalian cell lines available as hosts for expression are known in the art and include many immortalized cell lines available from the American Type Culture Collection (ATCC, Manassas, Va.), such as HeLa cells, Chinese hamster ovary (CHO) cells, baby hamster kidney (BHK) cells, and a number of other cell lines, particularly cell lines derived from hematopoietic and lymphopoietic cells.

Suitable promoters for mammalian cells are also known in the art and include viral promoters, such as those from Simian Virus 40 (SV40) (Fiers et al., Nature 273: 113 (1978)), Rous sarcoma virus (RSV), adenovirus (ADV), cytomegalovirus (CMV), and bovine papilloma virus (BPV), as well as mammalian cell-derived promoters. An exemplary, non-limiting list includes: a hematopoietic stem cell-specific promoter, such as the CD34 promoter (Burn et al., U.S. Pat. No. 5,556,954); the glucose-6-phosphotase promoter (Yoshiuchi et al., J. Clin. Endocrin. Metab. 83:1016-1019 (1998)); interleukin-1 alpha promoter (Mori and Prager, Leuk. Lymphoma 26:421-433 (1997)); CMV promoter (Tong et al., Anticancer Res. 18: 719-725 (1998), Norman et al., Vaccine 15:801-803 (1997)); RSV promoter (Elshami et al., Cancer Gene Ther. 4:213-221 (1997), Baldwin et al., Gene Ther. 4:1142-1149 (1997)); SV40 promoter (Harms and Splitter, Hum. Gene Ther. 6:1291-1297 (1995)); CD11c integrin gene promoter (Corbi and Lopez-Rodriguez, Leuk. Lymphoma 25:415-425 (1997)), GM-CSF promoter (Shannon et al., Crit. Rev. Immunol. 17:301-323 (1997)); interleukin-5R alpha promoter (Sun et al., Curr. Top. Microbiol. Immunol 211:173-187 (1996)); interleukin-2 promoter (Serfing et al., Biochim. Biophys. Acta 1263:181-200 (1995); O'Neill et al., Transplant Proc. 23:2862-2866 (1991)); c-fos promoter (Janknecht, Immunobiology 193:137-142 (1995), Janknecht et al., Carcinogenesis 16:443-450 (1995), Takai et al., Princess Takamatsu Symp. 22:197-204 (1991)); h-ras promoter (Rachal et al., EXS 64:330-342 (1993)); and DMD gene promoter (Ray et al., Adv. Exp. Med. Biol. 280:107-111 (1990)). All of the above documents are incorporated by reference in their entirety and can be relied on to make or use aspects of this invention, especially in designing and constructing appropriate vector and host expression systems.

Vectors used in mammalian cell expression systems may also include additional functional sequences. For example, terminator sequences, poly-A addition sequences, and internal ribosome entry site (IRES) sequences. Enhancer sequences, which increase expression, may also be included and sequences that promote amplification of the gene may also be desirable (for example, methotrexate resistance genes). One of skill in the art is familiar with numerous examples of these additional functional sequences, as well as other functional sequences, that may optionally be included in an expression vector.

Vectors suitable for replication in mammalian cells may include viral replicons, or sequences which insure integration of the appropriate sequences into the host genome. For example, another vector used to express foreign DNA is vaccinia virus. In this case, a nucleic acid molecule encoding an gene homologue or fragment thereof is inserted into the vaccinia genome. Techniques for the insertion of foreign DNA into the vaccinia virus genome are known in the art, and may utilize, for example, homologous recombination. Such heterologous DNA is generally inserted into a gene, which is non-essential to the virus. An example of such a gene is thymidine kinase (tk), which can also be used as a selectable marker. Plasmid vectors that greatly facilitate the construction of recombinant viruses have been described (see, for example, Mackett et al, J Virol. 49: 857 (1984); Chakrabarti et al., Mol. Cell. Biol. 5: 3403 (1985); Moss, In: Gene Transfer Vectors For Mammalian Cells (Miller and Calos, eds., Cold Spring Harbor Laboratory, N.Y., p. 10, (1987); all of which are herein incorporated by reference in their entirety). Expression of the polypeptide then occurs in cells or animals, which are infected with the live recombinant vaccinia virus.

The BHK-21 cell line is obtained from the ATCC (Rockville, Md.). The cells are cultured in Dulbecco's modified Eagle media (DMEM/high-glucose), supplemented to 2 mM (mM) L-glutamine and 10% fetal bovine serum (FBS). This formulation is designated BHK growth media. Selective media is BHK growth media supplemented with 453 units/mL hygromycin B (Calbiochem, San Diego, Calif.). The BHK-21 cell line is stably transfected with the HSV transactivating protein VP16, which transactivates the IE110 promoter found on the plasmid pMON3359 (See Hippenmeyer et al., Bio/Technology 11: 1037-1041 (1993), incorporated by reference in its entirety). The VP16 protein drives expression of genes inserted behind the IE110 promoter. BHK-21 cells expressing the transactivating protein VP16 are designated BHK-VP16. The plasmid pMON1118 (See Highkin et al., Poultry Sci. 70:970-981 (1991), incorporated by reference in its entirety) expresses the hygromycin resistance gene from the SV40 promoter. A similar plasmid, available from ATCC, is pSV2-hph.

The sequence to be integrated into the mammalian sequence may be introduced into the primary host by any convenient means, including calcium-phosphate precipitated DNA, spheroplast fusion, transformation, electroporation, biolistics, lipofection, microinjection, or other convenient means. Where an amplifiable gene is being employed, the amplifiable gene may serve as the selection marker for selecting hosts into which the amplifiable gene has been introduced. Alternatively, one may include with the amplifiable gene another marker, such as a drug resistance marker, e.g. neomycin resistance (G418 in mammalian cells), hygromycin resistance etc., or an auxotrophy marker (HIS3, TRP1, LEU2, URA3, ADE2, LYS2, etc.) for use in yeast cells.

Depending upon the nature of the modification and associated targeting construct, various techniques may be employed for identifying targeted integration. Conveniently, the DNA may be digested with one or more restriction enzymes and the fragments probed with an appropriate DNA fragment, which will identify the properly sized restriction fragment associated with integration.

One may use different promoter sequences, enhancer sequences, or other sequence which will allow for enhanced levels of expression in the expression host. Thus, one may combine an enhancer from one source, a promoter region from another source, a 5′-noncoding region upstream from the initiation methionine from the same or different source as the other sequences, and the like. One may provide for an intron in the non-coding region with appropriate splice sites or for an alternative 3′-untranslated sequence or polyadenylation site. Depending upon the particular purpose of the modification, any of these sequences may be introduced, as desired.

Where selection is intended, the sequence to be integrated will have an associated marker gene, which allows for selection. The marker gene may conveniently be downstream from the target gene and may include resistance to a cytotoxic agent, e.g. antibiotics, heavy metals, resistance or susceptibility to HAT, gancyclovir, etc., complementation to an auxotrophic host, particularly by using an auxotrophic yeast as the host for the subject manipulations, or the like. The marker gene may also be on a separate DNA molecule, particularly with primary mammalian cells. Alternatively, one may screen the various transformants, due to the high efficiency of recombination in yeast, by using hybridization analysis, PCR, sequencing, or the like.

For homologous recombination, constructs can be prepared where the amplifiable gene will be flanked, normally on both sides, with DNA homologous with the DNA of the target region. Depending upon the nature of the integrating DNA and the purpose of the integration, the homologous DNA will generally be within 100 kb, usually 50 kb, preferably about 25 kb, of the transcribed region of the target gene, more preferably within 2 kb of the target gene. Where modeling of the gene is intended, homology will usually be present proximal to the site of the mutation. The term gene is intended to encompass the coding region and those sequences required for transcription of a mature mRNA. The homologous DNA may include the 5′-upstream region outside of the transcriptional regulatory region, or comprise any enhancer sequences, transcriptional initiation sequences, adjacent sequences, or the like. The homologous region may include a portion of the coding region, where the coding region may be comprised only of an open reading frame or combination of exons and introns. The homologous region may comprise all or a portion of an intron, where all or a portion of one or more exons may also be present. Alternatively, the homologous region may comprise the 3′-region, so as to comprise all or a portion of the transcriptional termination region, or the region 3′ of this position. The homologous regions may extend over all or a portion of the target gene or be outside the target gene comprising all or a portion of the transcriptional regulatory regions and/or the structural gene.

The integrating constructs may be prepared in accordance with conventional methods, where sequences may be synthesized, isolated from natural sources, manipulated, cloned, ligated, subjected to in vitro mutagenesis, primer repair, or the like. At various stages, the joined sequences may be cloned and analyzed by restriction analysis, sequencing, or by similar methods. Usually during the preparation of a construct where various fragments are joined, the fragments, intermediate constructs and constructs will be carried on a cloning vector comprising a replication system functional in a prokaryotic host, e.g., E. coli, and a marker for selection, e.g., biocide resistance, complementation to an auxotrophic host, etc. Other functional sequences may also be present, such as polylinkers, for ease of introduction and excision of the construct or portions thereof, or the like. A large number of cloning vectors are available such as pBR322, the pUC series, etc. These constructs may then be used for integration into the primary mammalian host.

In the case of the primary mammalian host, a replicating vector may be used. Usually, such vector will have a viral replication system, such as SV40, bovine papilloma virus, adenovirus, or a comparable viral system. The linear DNA sequence vector may also have a selectable marker for identifying transfected cells. Selectable markers include the neo gene, allowing for selection with G418, the herpes tk gene for selection with HAT medium, the gpt gene with mycophenolic acid, complementation of an auxotrophic host, etc.

The vector may or may not be capable of stable maintenance in the host. Where the vector is capable of stable maintenance, the cells will be screened for homologous integration of the vector into the genome of the host, where various techniques for curing the cells may be employed. Where the vector is not capable of stable maintenance, for example, where a temperature sensitive replication system is employed, one may change the temperature from the permissive temperature to the non-permissive temperature so that the cells may be cured of the vector. In this case, only those cells having integration of the construct comprising the amplifiable gene and, when present, the selectable marker, will be able to survive selection.

Where a selectable marker is present, one may select for the presence of the targeting construct by means of the selectable marker. Where the selectable marker is not present, one may select for the presence of the construct by the amplifiable gene. For the neo gene or the herpes thymidinekinase (tk) gene, one could employ a medium for growth of the transformants of about 0.1-1 mg/ml of G418 or may use HAT medium, respectively. Where DHFR is the amplifiable gene, the selective medium may include from about 0.01-0.5 μM of methotrexate or be deficient in glycine-hypoxanthine-thymidine and have dialysed serum (GHT media).

The DNA can be introduced into the expression host by a variety of techniques that include calcium phosphate/DNA co-precipitates, microinjection of DNA into the nucleus, electroporation, yeast protoplast fusion with intact cells, transfection, polycations, e.g., polybrene, polyornithine, or the like. The DNA may be single- or double-stranded DNA. It may be linear or circular. The various techniques for transforming mammalian cells are well known (see Keown et al., Methods Enzymol. (1989), Keown et al., Methods Enzymol. 185:527-537 (1990); Mansour et al., Nature 336:348-352, (1988); all of which are herein incorporated by reference in their entirety).

(e) Insect Constructs and Transformed Insect Cells

The present invention also relates to an insect recombinant expression vector comprising exogenous genetic material. The present invention also relates to an insect cell comprising an insect recombinant vector. The present invention also relates to methods for obtaining a recombinant insect host cell, comprising introducing into an insect cell exogenous genetic material.

The insect recombinant vector may be any vector, which can be conveniently subjected to recombinant DNA procedures and can bring about the expression of the nucleic acid sequence. The choice of a vector will typically depend on the compatibility of the vector with the insect host cell into which the vector is to be introduced. The vector may be a linear or a closed circular plasmid. The vector system may be a single vector or plasmid, or two or more vectors or plasmids, which together contain the total DNA to be introduced into the genome of the insect host. In addition, the insect vector may be an expression vector.

Nucleic acid molecules can be inserted into a replication vector for expression in the insect cell under a suitable promoter for insect cells. Many vectors are available for this purpose, and selection of the appropriate vector will depend mainly on the size of the nucleic acid molecule to be inserted into the vector and the particular host cell to be transformed with the vector. Each vector contains various components depending on its function (amplification of DNA or expression of DNA) and the particular host cell with which it is compatible. The vector components for insect cell transformation generally include, but are not limited to, one or more of the following: a signal sequence, an origin of replication, one or more marker genes, and an inducible promoter.

The insect vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the insect cell, is integrated into the genome and replicated together with the chromosome(s) into which it has been integrated. For integration, the vector may rely on the nucleic acid sequence of the vector for stable integration into the genome by homologous or nonhomologous recombination.

Alternatively, the vector may contain additional nucleic acid sequences for directing integration by homologous recombination into the genome of the insect host. The additional nucleic acid sequences enable the vector to be integrated into the host cell genome at a precise location(s) in the chromosome(s). To increase the likelihood of integration at a precise location, there should be preferably two nucleic acid-sequences which individually contain a sufficient number of nucleic acids, preferably 400 bp to 1500 bp, more preferably 800 bp to 1000 bp, which are highly homologous with the corresponding target sequence to enhance the probability of homologous recombination. These nucleic acid sequences may be any sequence that is homologous with a target sequence in the genome of the insect host cell, and, furthermore, may be non-encoding or encoding sequences.

Baculovirus expression vectors (BEVs) have become important tools for the expression of foreign genes, both for basic research and for the production of proteins with direct clinical applications in human and veterinary medicine (Doerfler, Curr. Top. Microbiol. Immunol. 131: 51-68 (1968); Luckow and Summers, Bio/Technology 6: 47-55 (1988a); Miller, Annual Review of Microbiol. 42: 177-199 (1988); Summers, Curr. Comm. Molecular Biology, Cold Spring Harbor Press, Cold Spring Harbor, N.Y. (1988), all of which are herein incorporated by reference in their entirety). BEVs are recombinant insect viruses in which the coding sequence for a chosen foreign gene has been inserted behind a baculovirus promoter in place of the viral gene, e.g., polyhedrin (Smith and Summers, U.S. Pat. No. 4,745,051, the entirety of which is incorporated herein by reference).

The use of baculovirus vectors relies upon the host cells being derived from Lepidopteran insects such as Spodoptera frugiperda or Trichoplusia ni. The preferred Spodoptera frugiperda cell line is the cell line Sf9. The Spodoptera frugiperda Sf9 cell line was obtained from American Type Culture Collection (Manassas, Va.) and is assigned accession number ATCC CRL 1711. (Summers and Smith, A Manual of Methods for Baculovirus Vectors and Insect Cell Culture Procedures, Texas Ag. Exper. Station Bulletin No. 1555 (1988), the entirety of which is herein incorporated by reference). Other insect cell systems, such as the silkworm B. mori, may also be used.

The proteins expressed by the BEVs are, therefore, synthesized, modified and transported in host cells derived from Lepidopteran insects. Most of the genes that have been inserted and produced in the baculovirus expression vector system have been derived from vertebrate species. Other baculovirus genes in addition to the polyhedrin promoter may be employed to advantage in a baculovirus expression system. These include immediate-early (alpha), delayed-early (beta), late (gamma), or very late (delta), according to the phase of the viral infection during which they are expressed. The expression of these genes occurs sequentially, probably as the result of a “cascade” mechanism of transcriptional regulation. (Guarino and Summers, J. Virol. 57:563-571 (1986); Guarino and Summers, J. Virol. 61:2091-2099 (1987); Guarino and Summers, Virol. 162:444-451 (1988); all of which are herein incorporated by reference in their entirety).

Insect recombinant vectors are useful as an intermediate for the infection or transformation of insect cell systems. For example, an insect recombinant vector containing a nucleic acid molecule encoding a baculovirus transcriptional promoter followed downstream by an insect signal DNA sequence is capable of directing the secretion of the desired biologically active protein from the insect cell. The vector may utilize a baculovirus transcriptional promoter region derived from any of the over 500 baculoviruses generally infecting insects, such as for example, the Orders Lepidoptera, Diptera, Orthoptera, Coleoptera and Hymenoptera, including, for example, but not limited to the viral DNAs of Autographa californica MNPV, Bombyx mori NPV, Trichoplusia ni MNPV, Rachiplusia ou MNPV or Galleria mellonella MNPV, wherein said baculovirus transcriptional promoter is a baculovirus immediate-early gene IEl or IEN promoter; an immediate-early gene in combination with a baculovirus delayed-early gene promoter region selected from the group consisting of 39K and a HindIII-k fragment delayed-early gene; or a baculovirus late gene promoter. The immediate-early or delayed-early promoters can be enhanced with transcriptional enhancer elements. The insect signal DNA sequence may code for a signal peptide of a Lepidopteran adipokinetic hormone precursor or a signal peptide of the Manduca sexta adipokinetic hormone precursor (Summers, U.S. Pat. No. 5,155,037; the entirety of which is herein incorporated by reference). Other insect signal DNA sequences include a signal peptide of the Orthoptera Schistocerca gregaria locust adipokinetic hormone precursor and the Drosophila melanogaster cuticle genes CP1, CP2, CP3 or CP4 or for an insect signal peptide having substantially a similar chemical composition and function (Summers, U.S. Pat. No. 5,155,037).

Insect cells are distinctly different from animal cells. Insects have a unique life cycle and have distinct cellular properties such as the lack of intracellular plasminogen activators in insect cells, which are present in vertebrate cells. Another difference is the high expression levels of protein products ranging from 1 to greater than 500 mg/liter and the ease at which cDNA can be cloned into cells (Frasier, In Vitro Cell. Dev. Biol. 25:225 (1989); Summers and Smith, In: A Manual of Methods for Baculovirus Vectors and Insect Cell Culture Procedures, Texas Ag. Exper. Station Bulletin No. 1555 (1988), both of which are incorporated by reference in their entirety).

Recombinant protein expression in insect cells is achieved by viral infection or stable transformation. For viral infection, the desired gene is cloned into baculovirus at the site of the wild-type polyhedrin gene (Webb and Summers, Technique 2:173 (1990); Bishop and Posse, Adv. Gene Technol. 1:55 (1990); both of which are incorporated by reference in their entirety). The polyhedrin gene is a component of a protein coat in occlusions, which encapsulate virus particles. Deletion or insertion in the polyhedrin gene results in the failure to form occlusion bodies. Occlusion negative viruses are morphologically different from occlusion positive viruses and enable one skilled in the art to identify and purify recombinant viruses.

The vectors of present invention preferably contain one or more selectable markers, which permit easy selection of transformed cells. A selectable marker is a gene the product of which provides, for example, biocide or viral resistance, resistance to heavy metals, prototrophy to auxotrophs, and the like. Selection may be accomplished by co-transformation, e.g., as described in WO 91/17243, a nucleic acid sequence of the present invention may be operably linked to a suitable promoter sequence. The promoter sequence is a nucleic acid sequence, which is recognized by the insect host cell for expression of the nucleic acid sequence. The promoter sequence contains transcription and translation control sequences, which mediate the expression of the protein or fragment thereof. The promoter may be any nucleic acid sequence, which shows transcriptional activity in the insect host cell of choice and may be obtained from genes encoding polypeptides either homologous or heterologous to the host cell.

For example, a nucleic acid molecule encoding an homologue or fragment thereof may also be operably linked to a suitable leader sequence. A leader sequence is a nontranslated region of a mRNA, which is important for translation by the fungal host. The leader sequence is operably linked to the 5′ terminus of the nucleic acid sequence encoding the protein or fragment thereof. The leader sequence may be native to the nucleic acid sequence encoding the protein or fragment thereof or may be obtained from foreign sources. Any leader sequence, which is functional in the insect host cell of choice, may be used in the present invention.

A polyadenylation sequence may also be operably linked to the 3′ terminus of the nucleic acid sequence of the present invention. The polyadenylation sequence is a sequence which when transcribed is recognized by the insect host to add polyadenosine residues to transcribed mRNA. The polyadenylation sequence may be native to the nucleic acid sequence encoding the protein or fragment thereof or may be obtained from foreign sources. Any polyadenylation sequence, which is functional in the fungal host of choice, may be used in the present invention.

To avoid the necessity of disrupting the cell to obtain the protein or fragment thereof, and to minimize the amount of possible degradation of the expressed polypeptide within the cell, it is preferred that expression of the polypeptide gene gives rise to a product secreted outside the cell. To this end, the protein or fragment thereof of the present invention may be linked to a signal peptide which is in turn linked to the amino terminus of the protein or fragment thereof. A signal peptide is an amino acid sequence that permits the secretion of the protein or fragment thereof from the insect host into the culture medium. The signal peptide may be native to the protein or fragment thereof of the invention or may be obtained from foreign sources. The 5′ end of the coding sequence of the nucleic acid sequence of the present invention may inherently contain a signal peptide coding region naturally linked in the translation reading frame with the segment of the coding region that encodes the secreted protein or fragment thereof.

At present, a mode of achieving secretion of a foreign gene product in insect cells is by way of the foreign gene's native signal peptide. Because the foreign genes are usually from non-insect organisms, their signal sequences may be poorly recognized by insect cells, and hence, levels of expression may be suboptimal. However, the efficiency of expression of foreign gene products seems to depend primarily on the characteristics of the foreign protein. On average, nuclear localized or non-structural proteins are most highly expressed, secreted proteins are intermediate, and integral membrane proteins are the least expressed.

One factor generally affecting the efficiency of the production of foreign gene products in a heterologous host system is the presence of native signal sequences (also termed presequences, targeting signals, or leader sequences) associated with the foreign gene. The signal sequence is generally coded by a DNA sequence immediately following (5′ to 3′) the translation start site of the desired foreign gene.

The expression dependence on the type of signal sequence associated with a gene product can be represented by the following example: If a foreign gene is inserted at a site downstream from the translational start site of the baculovirus polyhedrin gene so as to produce a fusion protein (containing the N-terminus of the polyhedrin structural gene), the fused gene is highly expressed. But less expression is achieved when a foreign gene is inserted in a baculovirus expression vector immediately following the transcriptional start site and totally replacing the polyhedrin structural gene.

Insertions into the region −50 to −1 significantly alter (reduce) steady state transcription which, in turn, reduces translation of the foreign gene product. Use of the pVL941 vector, for example, optimizes transcription of foreign genes to the level of the polyhedrin gene transcription. Even though the transcription of a foreign gene may be optimal, optimal translation may vary because of several factors involving processing: signal peptide recognition, mRNA and ribosome binding, glycosylation, disulfide bond formation, sugar processing, oligomerization, for example.

The properties of the insect signal peptide are expected to be more optimal for the efficiency of the translation process in insect cells than those from vertebrate proteins. This phenomenon can generally be explained by the fact that proteins secreted from cells are synthesized as precursor molecules containing hydrophobic N-terminal signal peptides. The signal peptides direct transport of the select protein to its target membrane and are then cleaved by a peptidase on the membrane, such as the endoplasmic reticulum, when the protein passes through it.

Another exemplary insect signal sequence is the sequence encoding for Drosophila cuticle proteins such as CP1, CP2, CP3 or CP4 (Summers, U.S. Pat. No. 5,278,050; the entirety of which is herein incorporated by reference). Most of the 9 kb region of the Drosophila genome contain genes for the cuticle proteins has been sequenced. Four of the five cuticle genes contain a signal peptide coding sequence interrupted by a short intervening sequence (about 60 base pairs) at a conserved site. Conserved sequences occur in the 5′ mRNA untranslated region, in the adjacent 35 base pairs of upstream flanking sequence and at −200 base pairs from the mRNA start position in each of the cuticle genes.

Standard methods of insect cell culture, cotransfection and preparation of plasmids are set forth in Summers and Smith (Summers and Smith, A Manual of Methods for Baculovirus Vectors and Insect Cell Culture Procedures, Texas Agricultural Experiment Station Bulletin No. 1555, Texas A&M University (1987)). Procedures for the cultivation of viruses and cells are described in Volkman and Summers, J. Virol 19: 820-832 (1975) and Volkman et al., J. Virol 19: 820-832 (1976); both of which are herein incorporated by reference in their entirety.

Alternatively, recombinant baculoviruses can be created using a baculovirus shuttle vector system (Luckow et al., J. Virol. 67: 4566-4579 (1993), incorporated by reference in its entirety), now marketed as the Bac-To-Bac™ Expression System (Life Technologies, Inc. Rockville, Md.). Pure recombinant baculovirus carrying the recombinant gene is used to infect cells cultured, for example, in Excell 401 serum-free medium (JRH Biosciences, Lenexa, Kans.) or Sf900-II (Life Technologies, Inc.). The recombinant proteins secreted into the medium, for example, can be recovered by standard biochemical approaches. Supernatants from mammalian or insect cells expressing the recombinant proteins can be first concentrated using any of a number of commercial concentration units.

(f) Bacterial Constructs and Transformed Bacterial Cells

The present invention also relates to a bacterial recombinant vector comprising exogenous genetic material. The present invention also relates to a bacteria cell comprising a bacterial recombinant vector. The present invention also relates to methods for obtaining a recombinant bacteria host cell, comprising introducing into a bacterial host cell exogenous genetic material.

The bacterial recombinant vector may be any vector that can be conveniently subjected to recombinant DNA procedures. The choice of a vector will typically depend on the compatibility of the vector with the bacterial host cell into which the vector is to be introduced. The vector may be a linear or a closed circular plasmid. The vector system may be a single vector or plasmid or two or more vectors or plasmids that together contain the total DNA to be introduced into the genome of the bacterial host. In addition, the bacterial vector may be an expression vector. Nucleic acid molecules encoding gene homologues or fragments thereof can, for example, be suitably inserted into a replicable vector for expression in the bacterium under the control of a suitable promoter for bacteria. Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more marker genes, and an inducible promoter.

In general, plasmid vectors containing replicon and control sequences that are derived from species compatible with the host cell are used in connection with bacterial hosts. The vector ordinarily carries a replication site, as well as marking sequences that are capable of providing phenotypic selection in transformed cells. For example, E. coli is typically transformed using pBR322, a plasmid derived from an E. coli species (see, e.g., Bolivar et al., Gene 2: 95 (1977); the entirety of which is herein incorporated by reference). pBR322 contains genes for ampicillin and tetracycline resistance and thus provides easy means for identifying transformed cells. The pBR322 plasmid, or other microbial plasmid or phage, also generally contains, or is modified to contain, promoters that can be used by the microbial organism for expression of the selectable marker genes.

Nucleic acid molecules encoding gene homologues or fragments thereof may be expressed not only directly, but also as a fusion with another polypeptide, preferably a signal sequence or other polypeptide having a specific cleavage site at the N-terminus of the mature polypeptide. In general, the signal sequence may be a component of the vector, or it may be a part of the polypeptide DNA that is inserted into the vector. The heterologous signal sequence selected should be one that is recognized and processed (i.e., cleaved by a signal peptidase) by the host cell. For bacterial host cells that do not recognize and process the native polypeptide signal sequence, the signal sequence is substituted with a bacterial signal sequence selected, for example, from the group consisting of the alkaline phosphatase, penicillinase, lpp, or heat-stable enterotoxin II leaders.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria. The origin of replication from the plasmid pBR322 is suitable for most Gram-negative bacteria.

Expression and cloning vectors also generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene homologue or fragment thereof produce a protein conferring drug resistance and thus survive the selection regimen.

The expression vector for producing a polypeptide can also contains an inducible promoter that is recognized by the host bacterial organism and is operably linked to the nucleic acid encoding, for example, the gene homologue or fragment thereof of interest. Inducible promoters suitable for use with bacterial hosts include the beta-lactamase and lactose promoter systems (Chang et al., Nature 275: 615 (1978); Goeddel et al., Nature 281: 544 (1979); both of which are herein incorporated by reference in their entirety), the arabinose promoter system (Guzman et al., J. Bacteriol. 174: 7716-7728 (1992); the entirety of which is herein incorporated by reference), alkaline phosphatase, a tryptophan (trp) promoter system (Goeddel, Nucleic Acids Res. 8: 4057 (1980); EP 36,776; both of which are herein incorporated by reference in their entirety) and hybrid promoters such as the tac promoter (deBoer et al., Proc. Natl. Acad. Sci. USA 80: 21-25 (1983); the entirety of which is herein incorporated by reference). However, other known bacterial inducible promoters are suitable (Siebenlist et al., Cell 20:269 (1980); the entirety of which is herein incorporated by reference).

Promoters for use in bacterial systems also generally contain a Shine-Dalgarno (S.D.) sequence operably linked to the DNA encoding the polypeptide of interest. The promoter can be removed from the bacterial source DNA by restriction enzyme digestion and inserted into the vector containing the desired DNA.

Construction of suitable vectors containing one or more of the above-listed components employs standard ligation techniques. Isolated plasmids or DNA fragments are cleaved, tailored, and re-ligated in the form desired to generate the plasmids required. Examples of available bacterial expression vectors include, but are not limited to, the multifunctional E. coli cloning and expression vectors such as Bluescript™(Stratagene, La Jolla, Calif.), in which, for example, encoding an gene homologue or fragment thereof homologue, may be ligated into the vector in frame with sequences for the amino-terminal Met and the subsequent 7 residues of beta-galactosidase so that a hybrid protein is produced; pIN vectors (Van Heeke and Schuster J. Biol. Chem. 264: 5503-5509 (1989), the entirety of which is herein incorporated by reference); and others. pGEX vectors (Promega, Madison Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin, or factor Xa protease cleavage sites so that the cloned polypeptide of interest can be released from the GST moiety at will.

Species suitable as host bacteria for a bacterial vector include archaebacteria and eubacteria, especially eubacteria, and most preferably Enterobacteriaceae. Examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. Suitable E. coli hosts include E. coli W3110 (American Type Culture Collection (ATCC), Manassas, Va.) 27,325), E. coli 294 (ATCC 31,446), E. coli B, and E. coli X1776 (ATCC 31,537). These examples are illustrative rather than limiting. Mutant cells of any of the above-mentioned bacteria may also be employed. It is necessary to select the appropriate bacteria, taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. E. coli strain W3110 is a preferred host strain for recombinant DNA product fermentations. Preferably, the host cell should secrete minimal amounts of proteolytic enzymes.

Host cells are transfected and preferably transformed with the above-described vectors and cultured in conventional nutrient media modified as appropriate for inducing promoters, selecting transformants, or amplifying the genes encoding the desired sequences.

Numerous methods of transfection are known to the ordinarily skilled artisan, for example, calcium phosphate and electroporation. Depending on the host cell used, transformation is done using standard techniques appropriate to such cells. The calcium treatment employing calcium chloride, as described in section 1.82 of Sambrook et al., Molecular Cloning: A Laboratory Manual, New York: Cold Spring Harbor Laboratory Press, (1989), is generally used for bacterial cells that contain substantial cell-wall barriers. Another method for transformation employs polyethylene glycol/DMSO, as described in Chung and Miller (Chung and Miller, Nucleic Acids Res. 16: 3580 (1988); the entirety of which is herein incorporated by reference). Electroporation is another preferred method.

Bacterial cells used to produce the polypeptide of interest for purposes of this invention are cultured in suitable media in which the promoters for the nucleic acid encoding the heterologous polypeptide can be artificially induced as described generally, e.g., in Sambrook et al., Molecular Cloning: A Laboratory Manual, New York: Cold Spring Harbor Laboratory Press, (1989). Examples of suitable media are given in U.S. Pat. Nos. 5,304,472 and 5,342,763; both of which are incorporated by reference in their entirety.

D. Uses of the Agents of the Invention

1. Methods for Identifying Bioactive Proteins, Polypeptides, Fragments, or Variants of the Invention

Once the nucleic acid has been used to produce a protein, polypeptide, or a variant or fragment, any one of a number of assays can be used to identify bioactivity.

In addition, the agents of the invention are especially useful in high throughput screening methods. In general, these methods involve individual sample assay volumes less than about 250 μl, or more preferably less than about 100 μl. With smaller sample volumes, numerous individual assays can be performed simultaneously and via computer-operated instrumentation. The assays comprise the detectable interaction between a protein, polypeptide, fragment, nucleic acid, or antibody of the invention (sometimes referred to as the target) and an assay compound. Thus, the assays comprise two components, a target and an assay compound, where the assay compound may be part of a composition of multiple compounds. It is also possible for the agents of this invention to be used as assay compounds in screening methods where other proteins, polypeptides, nucleic acids, antibodies, or binding partners are the targets.

The assay compound can be selected from a library of small molecules, organic compounds which are either synthetic or natural, or mimetic libraries of randomized oligonucleotide-derived or peptide-derived compounds, for example. The compounds of the libraries may contain random chemical modifications, such as acylation, alkylation, esterification, amidation, or other modifications. Ideally, the largest number of separate structural entities will exist in a library that is tested against the agents of the invention for detectable interaction. A variety of other reagents may be used in the assay, such as buffers, salts, detergents, proteins, protease inhibitors, nuclease inhibitors, antimicrobial agents, or other reagents.

Detecting the interaction between the assay compound and the agent of the invention can be performed via a number of techniques. Fluorescence quenching, specific binding as with avidin-biotin, enzymatic activity, or inhibition of enzymatic activity are examples of the types of techniques used to detect interaction between two molecules. One of skill in the art can devise many specific assays depending on the activity sought. The type of assay used is not crucial to the use of this invention.

The screening methods may optionally employ a solid substrate to which one or more assay components are bound. Also, cell-based assays are often used in high throughput screening methods, so that the cell contains or expresses a component of the assay. Numerous permutations are possible.

Each of the activities listed below may be screened for, alone or in combination, in a method to detect an interaction with agents of the invention.

a. Inflammatory or Anti-Inflammatory Activity

Proteins or polypeptides of the present invention may also exhibit inflammatory or anti-inflammatory activity. These activities may relate to a stimulus to cells involved in the inflammatory response, inhibiting or promoting cell-cell interactions (for example, cell adhesion), inhibiting or promoting chemotaxis of cells involved in the inflammatory process, inhibiting or promoting cell extravasation, or stimulating or suppressing production of other factors, which more directly inhibit or promote an inflammatory response.

Proteins or polypeptides exhibiting anti-inflammatory activity or antibodies to inflammatory proteins or polypeptides can be used to treat atopic disorders and other inflammatory conditions including: chronic or acute inflammatory conditions, inflammation associated with infection, such as septic shock, sepsis or systemic inflammatory response syndrome (SIRS); ischemia-reperfusion injury; endotoxin lethality; arthritis; complement-mediated hyperacute rejection; nephritis; cytokine or chemokine-induced lung injury; inflammatory bowel disease; Crohn's disease; or disorders resulting from over-production of cytokines such as TNF or IL-1. Proteins or polypeptides or antibodies of the invention may also be useful to treat anaphylaxis and hypersensitivity to an antigenic substance or material.

b. Cytokine and Cell Growth or Differentiation Activity

A protein or polypeptide of the invention may exhibit cytokine, cell growth promoting or inhibiting, or cell differentiation promoting or inhibiting activity. Many protein factors secreted by immune cells, including cytokines, have exhibited activity in one or more factor dependent cell-based assays. These assays can be used to identify useful activities. The activity of a protein or polypeptide of the invention may be measured by the following methods or others known in the art.

Assays for T-cell or thymocyte proliferation include those described in Current Protocols in Immunology, Coligan, et al. Eds., Greene Publishing Associates and Wiley-Interscience (1994) (see, especially Chapter 3, In Vitro assays for Mouse Lymphocyte Function 3.1-3.19; Chapter 7, Immunologic studies in Humans); Takai et al., J. Immunol. 137:3494-3500 (1986); Bertagnolli et al., J. Immunol. 145:1706-1712 (1990); Bertagnolli et al., Cellular Immunology 133:327-341 (1991); Bertagnolli, et al., J. Immunol. 149:3778-3783 (1992); Bowman et al., J. Immunol. 152: 1756-1761 (1994).

Assays for cytokine production and/or proliferation of spleen cells, lymph node cells, or thymocytes include those described in Polyclonal T cell stimulation, Kruisbeek, and Shevach, In Current Protocols in Immunology, Coligan eds. Vol 1 pp. 3.12.1-3.12.14, John Wiley and Sons, Toronto (1994); and Measurement of mouse and human Interferon gamma, Schreiber, In Current Protocols in Immunology, Coligan eds. Vol 1 pp. 6.8.1-6.8.8, John Wiley and Sons, Toronto (1994).

Assays for proliferation and differentiation of hematopoietic and lymphopoietic cells include those described in Measurement of Human and Murine Interleukin 2 and Interleukin 4, Bottomly, Davis, and Lipsky In Current Protocols in Immunology. Coligan eds. Vol 1 pp. 6.3.1-6.3.12, John Wiley and Sons, Toronto (1994); deVries et al., J. Exp. Med. 173:1205-1211 (1991); Moreau et al., Nature 336:690-692 (1988); Greenberger et al., Proc. Natl. Acad. Sci. (U.S.A.) 80:2931-2938 (1983); Measurement of mouse and human interleukin 6, Nordan, R. In Current Protocols in Immunology. Coligan eds. Vol 1 pp. 6.6.1-6.6.5, John Wiley and Sons, Toronto (1994); Smith et al., Proc. Natl. Acad. Sci. (U.S.A.) 83:1857-1861 (1986); Measurement of human Interleukin 11, Bennett, et al., In Current Protocols in Immunology. Coligan eds. Vol 1 pp. 6.15.1 John Wiley and Sons, Toronto (1991); Measurement of mouse and human Interleukin 9, Ciarletta, et al., In Current Protocols in Immunology. Coligan eds. Vol 1 pp. 6.13.1, John Wiley and Sons, Toronto (1991).

Assays for T-cell clone responses to antigens (which will identify, among others, proteins that affect antigen-presenting cell (APC)-T cell interactions as well as direct T-cell effects by measuring proliferation and cytokine production) include, without limitation, those described in: Current Protocols in Immunology, Coligan, et al. eds., Pub. Greene Publishing Associates and Wiley-Interscience (1994)(Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. (U.S.A) 77:6091-6095, 1980; Weinberger et al., Eur. J. Immun. 11:405-411, 1981; Takai et al., J. Immunol. 137:3494-3500, 1986; Takai et al., J. Immunol. 140:508-512 (1988).

c. Immunosuppressive, Immune Stimulating, or Immune Modulating Activity

Suitable assays for thymocyte or splenocyte cytotoxicity include, without limitation, those described in: Current Protocols in Immunology, Ed by Coligan, et al., Pub. Greene Publishing Associates and Wiley-Interscience (1994)(Chapter 3, In Vitro assays for Mouse Lymphocyte Function 3.1-3.19; Chapter 7, Immunologic studies in Humans); Herrmann et al., Proc. Natl. Acad. Sci. (U.S.A.) 78:2488-2492 (1981); Herrmann et al., J. Immunol. 128:1968-1974 (1982); Handa et al., J. Immunol. 135:1564-1572 (1985); Takai et al., J. Immunol. 137:3494-3500 (1986); Takai et al., J. Immunol. 140:508-512 (1988); Bowman et al., J. Virology 61:1992-1998; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028:3033 (1990); and Assays for B cell function: In vitro antibody production, Mond, and Brunswick, In Current Protocols in Immunology. Coligan eds. Vol 1 pp. 3.8.1-3.8.16, John Wiley and Sons, Toronto (1994).

Mixed lymphocyte reaction (MLR) assays (which will identify, among others, proteins that generate predominantly Th1 and CTL responses) include, without limitation, those described in: Current Protocols in Immunology, Ed by Coligan, et al., Strober, Pub. Greene Publishing Associates and Wiley-Interscience (1994) (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783 (1992).

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., J Immunol 154:5071-5079 (1995); Porgador et al., Journal of Experimental Medicine 182:255-260 (1995); Nair et al., J. Virology 67:4062-4069 (1993); Huang et al., Science 264:961-965 (1994); Macatonia et al., Journal of Experimental Medicine 169:1255-1264 (1989); Bhardwaj et al., Journal of Clinical Investigation 94:797-807 (1994); and Inaba et al., Journal of Experimental Medicine 172:631-640 (1990).

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808 (1992); Gorczyca et al., Leukemia 7:659-670 (1993); Gorczyca et al., Cancer Research 53:1945-1951 (1993); Itoh et al., Cell 66:233-243 (1991); Zacharchuk, J. Immunol. 145:4037-4045 (1990); Zamai et al., Cytometry 14:891-897 (1993); Gorczyca et al., International Journal of Oncology 1:639-648 (1992).

Assays for proteins that influence early steps of T-cell commitment and development include, without limitation, those described in: Antica et al., Blood 84:111-117 (1994); Fine et al., Cellular Immunology 155:111-122 (1994); Galy et al., Blood 85:2770-2778 (1995); Toki et al., Proc. Nat. Acad Sci. (U.S.A.) 88:7548-7551 (1991).

d. Cell Differentiation Activity

Assays for embryonic stem cell differentiation (which will identify, among others, proteins that influence embryonic differentiation hematopoiesis) include, without limitation, those described in: Johansson et al., Cellular Biology 15:141-151 (1995); Keller et al., Molecular and Cellular Biology 13:473-486 (1993); McClanahan et al., 81:2903-2915 (1993).

Assays for stem cell survival and differentiation (which will identify, among others, proteins that regulate lympho-hematopoiesis) include, without limitation, those described in: Methylcellulose colony forming assays, Freshney, In Culture of Hematopoietic Cells. Freshney, et al., eds. Vol pp. 265-268, Wiley-Liss, Inc., New York, N.Y. (1994); Hirayama et al., Proc. Natl. Acad. Sci. (U.S.A.) 89:5907-5911 (1992); Primitive hematopoietic colony forming cells with high proliferate potential, McNiece, and Briddell, In Culture of Hematopoietic Cells, Freshney, et al. eds. Vol pp. 23-39, Wiley-Liss, Inc., New York, N.Y. (1994); Neben et al., Experimental Hematology 22:353-359 (1994); Cobblestone area forming cell assay, Ploemacher, In Culture of Hematopoietic Cells, Freshney, et al., eds. Vol pp. 1-21, Wiley-Liss, Inc., New York, N.Y. (1994); and Long term bone marrow cultures in the presence of stromal cells, Spooncer, et al., In Culture of Hematopoietic Cells, Freshney, et al., eds. Vol pp. 163-179, Wiley-Liss, Inc., New York, N.Y. (1994); Long term culture initiating cell assay, Sutherland, In Culture of Hematopoietic Cells, R. I. Freshney, et al., eds. Vol pp. 139-162, Wiley-Liss, Inc., New York, N.Y. (1994).

e. Wound Healing Activity

Assays for tissue generation activity include, without limitation, those described in: International Patent Publication No. WO95/16035 (bone, cartilage, tendon); International Patent Publication No. WO95/05846 (nerve, neuronal); and International Patent Publication No. WO91/07491 (skin, endothelium).

Assays for wound healing activity include, without limitation, those described in: Winter, Epidermal Wound Healing, pps. 71-112 (Maibach, and Rovee, eds.), Year Book Medical Publishers, Inc., Chicago, as modified by Eaglstein and Mertz, J. Invest. Dermatol 71:382-84 (1978).

f. Chemotactic Activity

A protein or peptide has chemotactic activity for a particular cell population if it can stimulate, directly or indirectly, the controlled orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by Coligan et al., Pub. Greene Publishing Associates and Wiley-Interscience (Chapter 6.12, Measurement of alpha and beta Chemokines 6.12.1-6.12.28; Taub et al., J. Clin. Invest. 95:1370-1376 (1995); Lind et al., APMIS 103:140-146 (1995); Muller et al., Eur. J. Immunol. 25: 1744-1748; Gruber et al., J. of Immunol. 152:5860-5867 (1994); and Johnston et al. J. of Immunol. 153: 1762-1768 (1994).

g. Receptor/Ligand Interaction Activity

Proteins of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). These proteins or fragments of the invention, or cells containing them, can be incorporated into an assay to screen for binding to extracellular matrix proteins, their analogs, or for receptor/ligand interaction to compounds implicated in binding to extracellular matrix proteins. In a particularly preferred embodiment, molecules containing the peptide motif RGD can be used to screen for interaction with the proteins, fragments, or cells of the invention. Various specific assays can be used a basis for designing the reagents for screening, such as phage attachment assays, panning assays, cell attachment assays, and inhibition of cell attachment/adhesion assays (Pasquelina et al., J. Cell Biol. 130:1189-1196 (1995), Koivunen et al., BioTechnology 13: 265-270 (1995), Koivunen et al., Methods Enzym. 245: 346-369 (1994), and U.S. Pat. No. 5,817,750, all incorporated herein in their entirety). These receptor/ligand interaction assays can also be designed for use with libraries of compounds, such as phage display libraries.

Receptors and ligands are also useful for screening of potential peptide or small molecule inhibitors of the relevant receptor/ligand interaction. Proteins of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by Coligan, et al., Pub. Greene Publishing Associates and Wiley-Interscience (Chapter 7.28, Measurement of Cellular Adhesion under Static Conditions 7.28.1-7.28.22); Takai et al., Proc. Natl. Acad. Sci. (U.S.A.) 84:6864-6868 (1987); Bierer et al., J. Exp. Med. 168:1145-1156 (1988); Rosenstein et al., J. Exp. Med. 169:149-160 (1989); Stoltenborg et al., J. Immunol. Methods 175:59-68, 1994; Stitt et al., Cell 80:661-670 (1995).

2. Methods for Detecting and Manipulating Nucleic Acids

The nucleic acids of the invention can be used directly in numerous methods to identify or detect the presence of specific nucleic acid sequences. As noted above, the nucleic acids of the invention can be used as hybridization probes or PCR probes, or to derive specific hybridization or PCR probes. Furthermore, the nucleic acids of the invention of variants or fragments thereof can be linked to solid supports. In this way, various microarrays, beads, glass or nylon slides, membranes or other repeatable assay apparati can be constructed. A non-limiting description of selected methods follows.

a. Microarrays

In one embodiment, the nucleic acids of the invention can be used to monitor expression. A microarray-based method for high-throughput monitoring of gene expression may be utilized to measure activated eosinophil-related hybridization targets. This ‘chip’-based approach involves using microarrays of nucleic acids as specific hybridization targets to quantitatively measure expression of the corresponding genes (Schena et al., Science 270:467-470 (1995), the entirety of which is herein incorporated by reference; Shalon, Ph.D. Thesis, Stanford University (1996), the entirety of which is herein incorporated by reference). Every nucleotide in a large sequence can be queried at the same time. Hybridization can also be used to efficiently analyze nucleotide sequences.

Several microarray methods have been described. One method compares the sequences to be analyzed by hybridization to a set of oligonucleotides or cDNA molecules representing all possible subsequences (Bains and Smith, J. Theor. Biol. 135:303 (1989), the entirety of which is herein incorporated by reference). A second method hybridizes the sample to an array of oligonucleotide or cDNA probes. An array consisting of oligonucleotides or cDNA molecules complementary to subsequences of a target sequence can be used to determine the identity of a target sequence, measure its amount, and detect differences between the target and a reference sequence. Nucleic acid microarrays may also be screened with protein molecules or fragments thereof to determine nucleic acids that specifically bind protein molecules or fragments thereof.

The microarray approach may also be used with polypeptide targets (see, U.S. Pat. Nos. 5,800,992, 5,445,934; 5,143,854, 5,079,600, 4,923,901, all of which are herein incorporated by reference in their entirety). Essentially, polypeptides are synthesized on a substrate (microarray) and these polypeptides can be screened with either protein molecules or fragments thereof or nucleic acid molecules in order to screen for either protein molecules or fragments thereof or nucleic acid molecules that specifically bind the target polypeptides (Fodor et al., Science 251:767-773 (1991), the entirety of which is herein incorporated by reference).

b. Hybridization Assays

Oligonucleotide probes, whose sequences are complementary to that of a portion of the nucleic acids of the invention, such as SEQ NO.:1-71, can be constructed. These probes are then incubated with cell extracts of a patient under conditions sufficient to permit nucleic acid hybridization. The detection of double-stranded probe-mRNA hybrid molecules is indicative of the presence of activated eosinohils or sequences derived from activated eosinophils. Thus, such probes may be used to ascertain the level and extent of eosinophil activation or the production of certain proteins. The nucleic acid hybridization may be conducted under quantitative conditions or as a qualitative assay.

c. PCR Assays

A nucleic acid of the invention, such as one of SEQ NO.:1-71 or complements thereof, can be analyzed for use as a PCR probe. A search of databases indicates the presence of regions within that nucleic acid that have high and low regions of identity to other sequences in the database. Ideally, a PCR probe will have high identity with only the sequence from which it is derived. In that way, only the desired sequence is amplified. Computer generated searches using programs such as MIT Primer3 (Rozen and Skaletsky (1996, 1997, 1998)), or GeneUp (Pesole, et al., BioTechniques 25:112-123 (1998)), for example, can be used to identify potential PCR primers.

The PCR probes or primers can be used in methods such as described in Krzesicki, et al., Am. J. Respir. Cell Mol. Biol. 16:693-701 (1997) (incorporated by reference in its entirety) to identify or detect sequences expressed in activated eosinophils.

d. Ligation and Alternative Amplification Methods and Identification of Polymorphisms

In one sub-aspect of the invention analysis is conducted to determine the presence and/or identity of polymorphism(s) using one or more of the nucleic acid molecules of the present invention and more specifically one or more of the EST nucleic acid molecule or fragment thereof which are associated with a phenotype, or a predisposition to that phenotype.

Any of a variety of molecules can be used to identify such polymorphism(s). In one embodiment, one or more of the EST nucleic acid molecules (or a sub-fragment thereof) may be employed as a marker nucleic acid molecule to identify such polymorphism(s). Alternatively, such polymorphisms can be detected through the use of a marker nucleic acid molecule or a marker protein that is genetically linked to (i.e., a polynucleotide that co-segregates with) such polymorphism(s).

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s), and more preferably within 100 kb of the polymorphism(s), and most preferably within 10 kb of the polymorphism(s) can be employed.

The genomes of animals and plants naturally undergo spontaneous mutation in the course of their continuing evolution (Gusella, Ann. Rev. Biochem. 55:831-854 (1986)). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist, and the polymorphism is thus said to be di-allelic. In other cases, the species' population may contain multiple alleles, and the polymorphism is termed tri-allelic, etc. A single gene may have multiple different unrelated polymorphisms. For example, it may have a di-allelic polymorphism at one site, and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour, et al., FEBS Lett. 307:113-115 (1992); Jones, et al., Eur. J. Haematol. 39:144-147 (1987); Horn, et al., PCT Patent Application WO91/14003; Jeffreys, European Patent Application 370,719; Jeffreys, U.S. Pat. No. 5,175,082; Jeffreys et al., Amer. J. Hum. Genet. 39:11-24 (1986); Jeffreys et al., Nature 316:76-79 (1985); Gray et al., Proc. R. Acad. Soc. Lond. 243:241-253 (1991); Moore et al., Genomics 10:654-660 (1991); Jeffreys et al., Anim. Genet. 18:1-15 (1987); Hillel et al., Anim. Genet. 20:145-155 (1989); Hillel et al., Genet. 124:783-789 (1990), all of which are herein incorporated by reference in their entirety).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

The most preferred method of achieving such amplification employs the polymerase chain reaction (“PCR”)(See above). In lieu of PCR, alternative amplification methods, such as the “Ligase Chain Reaction” (“LCR”) may be used (Barany, Proc. Natl. Acad. Sci. (U.S.A.) 88:189-193 (1991). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide. Alternatively, the oligonucleotides may be selected such that they do not include the polymorphic site (see, Segev, PCT Application WO 90/01069).

The “Oligonucleotide Ligation Assay” (“OLA”) may alternatively be employed (Landegren, et al., Science 241:1077-1080 (1988)). The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, et al. have described a nucleic acid detection assay that combines attributes of PCR and OLA (Nickerson, et al., Proc. Natl. Acad. Sci. (U.S.A.) 87:8923-8927 (1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide,” thereby amplifying the di-oligonucleotide, are also known (Wu, et al., Genomics 4:560 (1989)), and may be readily adapted to the purposes of the present invention.

Other known nucleic acid amplification procedures, such as allele-specific oligomers, branched DNA technology, transcription-based amplification systems, or isothermal amplification methods may also be used to amplify and analyze such polymorphisms (Malek, et al., U.S. Pat. No. 5,130,238; Davey, et al., European Patent Application 329,822; Schuster et al., U.S. Pat. No. 5,169,766; Miller, et al., PCT appln. WO 89/06700; Kwoh, et al., Proc. Natl. Acad. Sci. (U.S.A.) 86:1173 (1989); Gingeras, et al., PCT application WO 88/10315; Walker, et al., Proc. Natl. Acad. Sci. (U.S.A.) 89:392-396 (1992)). Any of the foregoing nucleic acid amplification methods could be used.

The identification of a polymorphism in a gene, fragment or cellular sequence derived from the nucleic acids of the invention can be determined in a variety of ways. By correlating the presence or absence of atopic disease, for example, in an individual with the presence or absence of a polymorphism, it is possible to diagnose the predisposition of a patient to eosinophil related disorders. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and animal genetic analyses (Glassberg, UK Patent Application 2135774; Skolnick, et al., Cytogen. Cell Genet. 32:58-67 (1982); Botstein, et al., Ann. J. Hum. Genet. 32:314-331 (1980); Fischer, et al. (PCT Application WO90/13668); Uhlen, PCT Application WO90/11369), all of which are herein incorporated by reference in their entirety).

Other types of polymorphisms include single nucleotide polymorphisms (SNPs) that are single base changes in genomic DNA sequence. They generally occur at greater frequency than other markers and are spaced with a greater uniformity throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a result of mutations or defective splicing). Analysis (genotyping) of characterized SNPs can require only a plus/minus assay rather than a lengthy measurement, permitting easier automation.

SNPs can be characterized using any of a variety of methods. Such methods include the direct or indirect sequencing of the site, the use of restriction enzymes (Botstein et al., Am. J. Hum. Genet. 32:314-331 (1980), the entirety of which is herein incorporated reference; Konieczny and Ausubel, Plant J. 4:403-410 (1993), the entirety of which is herein incorporated by reference), enzymatic and chemical mismatch assays (Myers et al., Nature 313:495-498 (1985), the entirety of which is herein incorporated by reference), allele-specific PCR (Newton et al., Nucl. Acids Res. 17:2503-2516 (1989), the entirety of which is herein incorporated by reference; Wu et al., Proc. Natl. Acad. Sci. USA 86:2757-2760 (1989), the entirety of which is herein incorporated by reference), ligase chain reaction (Barany, Proc. Natl. Acad. Sci. USA 88:189-193 (1991), the entirety of which is herein incorporated by reference), single-strand conformation polymorphism analysis (Labrune et al., Am. J. Hum. Genet. 48: 1115-1120 (1991), the entirety of which is herein incorporated by reference), primer-directed nucleotide incorporation assays (Kuppuswami et al., Proc. Natl. Acad. Sci. USA 88:1143-1147 (1991), the entirety of which is herein incorporated by reference), dideoxy fingerprinting (Sarkar et al., Genomics 13:441-443 (1992), the entirety of which is herein incorporated by reference), solid-phase ELISA-based oligonucleotide ligation assays (Nikiforov et al., Nucl. Acids Res. 22:4167-4175 (1994), the entirety of which is herein incorporated by reference), oligonucleotide fluorescence-quenching assays (Livak et al., PCR Methods Appl. 4:357-362 (1995a), the entirety of which is herein incorporated by reference), 5′-nuclease allele-specific hybridization TaqMan assay (Livak et al., Nature Genet. 9:341-342 (1995), the entirety of which is herein incorporated by reference), template-directed dye-terminator incorporation (TDI) assay (Chen and Kwok, Nucl. Acids Res. 25:347-353 (1997), the entirety of which is herein incorporated by reference), allele-specific molecular beacon assay (Tyagi et al., Nature Biotech. 16: 49-53 (1998), the entirety of which is herein incorporated by reference), PinPoint assay (Haff and Smirnov, Genome Res. 7: 378-388 (1997), the entirety of which is herein incorporated by reference), and dCAPS analysis (Neff et al., Plant J. 14:387-392 (1998), the entirety of which is herein incorporated by reference).

SNPs can be observed by examining sequences of overlapping clones in the BAC library according to the method described by Taillon-Miller et al. Genome Res. 8:748-754 (1998), the entirety of which is herein incorporated). SNPs can also be observed by screening the BAC library of the present invention by colony or plaque hybridization with a labeled probe containing SNP markers; isolating positive clones and sequencing the inserts of the positive clones; suitable primers flanking the SNP markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996), the entirety of which is herein incorporated by reference); Orita et al., Genomics 5:874-879 (1989), the entirety of which is herein incorporated by reference). Under denaturing conditions a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis. A number of protocols have been described for SSCP including, but not limited to, Lee et al., Anal. Biochem. 205:289-293 (1992), the entirety of which is herein incorporated by reference; Suzuki et al., Anal. Biochem. 192:82-84 (1991), the entirety of which is herein incorporated by reference; Lo et al., Nucleic Acids Research 20: 1005-1009 (1992), the entirety of which is herein incorporated by reference; and Sarkar et al., Genomics 13:441-443 (1992), the entirety of which is herein incorporated by reference. It is understood that one or more of the nucleic acids of the present invention, may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos et al., Nucleic Acids Res. 23:4407-4414 (1995), the entirety of which is herein incorporated by reference). This method allows for the specific co-amplification of high numbers of restriction fragments, which can be visualized by PCR without knowledge of the nucleic acid sequence.

AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel.

AFLP analysis has been performed on Salix (Beismann et al., Mol. Ecol. 6:989-993 (1997), the entirety of which is herein incorporated by reference), Acinetobacter (Janssen et al., Int. J. Syst. Bacteriol. 47:1179-1187 (1997), the entirety of which is herein incorporated by reference), Aeromonas popoffi (Huys et al., Int. J. Syst. Bacteriol. 47:1165-1171 (1997), the entirety of which is herein incorporated by reference), rice (McCouch et al., Plant Mol. Biol. 35:89-99 (1997), the entirety of which is herein incorporated by reference; Nandi et al., Mol. Gen. Genet. 255:1-8 (1997), the entirety of which is herein incorporated by reference; Cho et al., Genome 39:373-378 (1996), the entirety of which is herein incorporated by reference), barley (Hordeum vulgare)(Simons et al., Genomics 44:61-70 (1997), the entirety of which is herein incorporated by reference; Waugh et al., Mol. Gen. Genet. 255:311-321 (1997), the entirety of which is herein incorporated by reference; Qi et al., Mol. Gen. Genet. 254:330-336 (1997), the entirety of which is herein incorporated by reference; Becker et al., Mol. Gen. Genet. 249:65-73 (1995), the entirety of which is herein incorporated by reference), potato (Van der Voort et al., Mol. Gen. Genet. 255:438-447 (1997), the entirety of which is herein incorporated by reference; Meksem et al., Mol. Gen. Genet. 249:74-81 (1995), the entirety of which is herein incorporated by reference), Phytophthora infestans (Van der Lee et al., Fungal Genet. Biol. 21:278-291 (1997), the entirety of which is herein incorporated by reference), Bacillus anthracis (Keim et al., J. Bacteriol. 179:818-824 (1997), the entirety of which is herein incorporated by reference), Astragalus cremnophylax (Travis et al., Mol. Ecol. 5:735-745 (1996), the entirety of which is herein incorporated by reference), Arabidopsis (Cnops et al., Mol. Gen. Genet. 253:32-41 (1996), the entirety of which is herein incorporated by reference), Escherichia coli (Lin et al., Nucleic Acids Res. 24:3649-3650 (1996), the entirety of which is herein incorporated by reference), Aeromonas (Huys et al., Int. J. Syst. Bacteriol. 46:572-580 (1996), the entirety of which is herein incorporated by reference), nematode (Folkertsma et al., Mol. Plant Microbe Interact. 9:47-54 (1996), the entirety of which is herein incorporated by reference), tomato (Thomas et al., Plant J. 8:785-794 (1995), the entirety of which is herein incorporated by reference), and human (Latorra et al., PCR Methods Appl. 3:351-358 (1994), the entirety of which is herein incorporated by reference). AFLP analysis has also been used for fingerprinting mRNA (Money et al., Nucleic Acids Res. 24:2616-2617 (1996), the entirety of which is herein incorporated by reference; Bachem et al., Plant J. 9:745-753 (1996), the entirety of which is herein incorporated by reference). It is understood that one or more of the nucleic acids of the present invention, may be utilized as markers or probes to detect polymorphisms by AFLP analysis or for fingerprinting RNA.

The polymorphism obtained by these approaches can then be cloned to identify the mutation at the coding region, which alters the protein's structure or the regulatory region of the gene that affects its expression level. Changes involving promoter interactions with other regulatory proteins can be identified by, for example, gel shift.

In accordance with an embodiment of the invention, a sample DNA is obtained from a patient's cells. In a preferred embodiment, the DNA sample is obtained from the patient's blood. However, any source of DNA may be used. The DNA may be subjected to interrogation to determine the presence or absence of a polymorphism.

### EXAMPLES

The following examples will illustrate the invention in greater detail, although it will be understood that the invention is not limited to these specific examples. Various other examples will be apparent to the person skilled in the art after reading the present disclosure without departing from the spirit and scope of the invention. It is intended that all such other examples be included within the scope of the appended claims.

### Example 1

### High Throughput Screening Method for Cell Interaction/Adhesion Molecules

Cell adhesion assays are difficult in that the interactions at the molecular level are typically low-affinity, while at the cellular level, they demonstrate cooperative binding. Isolated adhesion molecules can be used in high throughput screens. An adhesion assay based on Scintillation Proximity Assays (SPA) has been described (Game et al., Analytical Biochemistry 258:127-135, (1998)). One of the protein binding pair is immobilized on a scintillant-coated bead while the other protein is radio-labeled. Binding to the bead is detected as scintillation decays. Recent advances in the scintillant beads enable their use in 1536-well plates.

A non-radiometric alternative to SPA is homogenous, time-resolved fluorescence (HTRF) which measures inter-molecular interactions with a long half-life fluorescence energy transfer.

### Example 2

### High Throughput Screening Method for Kinases

Several excellent methods are available for the detection of tyrosine kinase activity. All depend on a phosphotryosyl peptide-specific antibody (anti-P-Tyr) binding to the reaction product. Anti-P-Tyr antibodies are commercially available and can be used in conjunction with HTRF labeling for a homogenous screening assay (Kolb et al., Drug Discovery Today 3:333-342(1998)). The same assay format can be used for Ser/Thr kinases.

Fluorescence Polarization has also been employed for detection of tyrosine kinases where the native product protein competes for the anti-P-Tyr antibody with a tracer fluorescent P-Tyr peptide. This enables the use of the native substrate where peptide substrates do not have good kinetic properties (Ramakrishna and Menzel, Anal. Biochem. 255:257-262 (1998)).

### Example 3

### High Throughput Screening Method for Proteases

HTRF is also an appropriate format for protease assay screening, especially for low activity enzyme/substrate pairs. The energy transfer groups are synthesized on either side of the scissile bond and activity is measured as an increase in fluorescence (Devlin, ed., High Throughput Screening, Dekker Publishing, New York (1997)).

For protease/substrate pairs with good turnover, fluorescence polarization offers a robust and inexpensive method for screening for protease inhibitors. The method detects the change in molecular volume of a fluorescent substrate upon cleavage (Levine et al., Anal. Biochem. 247:83-88 (1997)).

### Example 4

### High Throughput Screening Method for G-Coupled Protein Receptors

Two assay methods are proposed depending on whether the GPCR is cloned into a host cell and can be coupled to a reporter enzyme or whether the GPCR is in its native membrane and is used as a membrane preparation. For whole cell assays, Aurora Biosciences, Inc. has linked reporter gene expression to GPCR signaling. They have in-licensed a promiscuous G-protein from CALTECH which enables them to couple b-lactamase expression to signaling by virtually any GPCR. They have cell lines that couple Gq->PLA2->Ca²⁺->NFAT->β-lactamase as well as Gs->cAMP->CRE->β-lactamase. One of the strengths of this technology is the ability to use fluorescence activated cell sorting (FACS) technology to rapidly (2-3 weeks) select stable, high expressing transfected cell line for use in the screens, thereby reducing assay development time. β-lactamase activity is detected by introduction of a cell-permeable fluorescent substrate (Zlokarnik et al., Science 279:84-88 (1998)).

Another method that is amenable to membrane preparations of G-protein coupled receptors is the use of scintillant-coated FlashPlates to detect the agonist-induced binding of GTP-γS to membranes or cells. FlashPlates scintillate only when isotope is bound to or near the plate surface. No separation of bound from free is needed (Watson et al., J. Biomolecular Screening 3:101-105 (1988)).

### Example 5

### High Throughput Screening Method for Transcription Factors

The detection of transcription factor activity is done by the use of reporter genes. As described for the GPCR assays, β-lactamase is a very sensitive indicator of gene activity. There is no β-lactamase activity in mammalian cells, which means that there is no background activity. The cell permeant substrate for β-lactamase enables a homogenous assay that can detect as little as 100 molecules of β-lactamase/cell (Zlokarnik et al., Science 279:84-88 (1998)).

Another reporter construct that is used is screening transcriptional targets is the secreted alkaline phosphatase (SEAP). This enzyme is secreted into the medium and is detected by the addition of a chemiluminescence substrate (Bronstein et al., BioTechniques 17:172-178 (1994)).

### Example 6

### Strategy for obtaining FL clones

The sequences, or fragments of them, disclosed can be used to directly obtain full length clones from cDNA libraries and genomic clones from genomic libraries. A number of methods have been described to obtain cDNA and genomic clones, including 5′ RACE (for example, using the Marathon cDNA Amplification Kit from Clonetech, Inc.), Genetrapper (LifeTechnologies, Inc.), colony hybridization (Sambrook et al., 1989, Molecular Cloning, A Laboratory Manual 2nd Ed., Cold Spring Harbor Press, Cold Spring Harbor, N.Y.) and array hybridization. One skilled in the art can refer to general reference texts for detailed descriptions of known techniques or equivalent techniques. These texts include Current Protocols in Molecular Biology (Ausubel, et al., eds., John Wiley & Sons, N.Y. (1989), and supplements through September 1998), Molecular Cloning, A Laboratory Manual (Sambrook et al., 2nd Ed., Cold Spring Harbor Press, Cold Spring Harbor, N.Y. (1989)), and Current Protocols in Immunology (Coligan, ed., John Wiley and Sons, Toronto (1994)), for example, each of which are specifically incorporated by reference in their entirety.

A preferred method is array hybridization. A first step in array hybridization is to obtain a high quality library with high complexity and a high proportion of full length or high molecular weight clones. A cDNA library can be purchased from a number of commercial sources or a new library prepared from mRNA derived from tissue known or suspected to express the gene. Details on library construction can be found in Sambrook et al. (Molecular Cloning, A Laboratory Manual 2nd Ed., Cold Spring Harbor Press, Cold Spring Harbor, N.Y. ). Similarly, genomic libraries may be purchased from commercial sources.

A plasmid cDNA library constructed from activated human eosinophil cell-derived mRNA is plated on agar and a picking robot (for example, a ‘Q’ BOT from Genetix, Inc.) is used to pick individual colonies into 96 well plates containing LB medium. The isolated E. coli cells are grown overnight at 30° C. and placed at 4° C. until ready for spotting. The E. coli are then robotically spotted in high density grids on nitrocellulose membranes overlaying solid agar medium. Preferably, each colony is double spotted at two different locations. The colonies are allowed to grow to 0.1-0.2 cm and then the membranes are prepared for hybridization to nucleic acid sequences. Standard protocols for membrane preparation can be found in Sambrook (Molecular Cloning, A Laboratory Manual 2nd Ed., Cold Spring Harbor Press, Cold Spring Harbor, N.Y.).

Selection of nucleic acid probes, or similarly PCR primers, can be done using a variety of methods, including publicly available programs such as MIT Primer 3 (Rozen and Skaletsky (1996, 1997, 1998)), for example. The sequence of SEQ NO: 46 (1746748 h integrin b7) was run through the program MIT Primer3 at the default settings. The following preferred PCR primers were identified by the program using default parameters:

| | | |
|---|---|---|
| Primer 1 | CAGACGGTGACTTTCTGGGT | (SEQ NO: 72) |
| Primer 2 | TGCAACTCCACAATCAGCTC | |

At the default settings, additional primer pairs were identified:

| | | |
|---|---|---|
| Primer 1 | AGAGGGAGGGTAAGGCTGAG | (SEQ NO: 73) |
| Primer 2 | TCCAACTCCACAATCACCTC | |
| Primer 1 | AGGGTCCTGAGAAGAGGGAG | (SEQ NO: 74) |
| Primer 2 | TGCAACTCCACAATCAGCTC | |
| Primer 1 | GAAGAGGGAGGGTAAGGCTG | (SEQ NO: 75) |
| Primer 2 | TGCAACTCCACAATCACCTC | |
| Primer 1 | AGGATCGAGGACAGTGCAAC | (SEQ NO: 76) |
| Primer 2 | TGCAACTCCACAATCAGCTC | |

The following preferred probe for hybridization was identified using the default parameters except the annealing temperature was specified to be 80C:

| | | |
|---|---|---|
| Probe 1 | GGCGCCCACCCGCACAGCTGCAT | (SEQ NO: 77) |

At the same settings, additional probes were identified:

| | |
|---|---|
| GCGGCCAGGGGCACAGCTGCAT | (SEQ NO: 78) |
| CCGACCTAGGCGGCCAGGGGCA | (SEQ NO: 79) |

Any of the above probe sequences can be used in this example. However, probe 1 is preferred.

Synthetic oligos having the sequence of probe 1 or a restriction fragment containing probe 1 sequence, from the original clone bearing the SEQ NO: 46 (h integrin b7) sequence, is labeled with ³³P using conventional means, such as the random hexamer priming method (High Prime kit from Boehringer Mannheim). The labeled probes are then hybridized to the arrayed filters under stringent conditions (Sambrook et al., 1989, Molecular Cloning, A Laboratory Manual 2nd Ed., Cold Spring Harbor Press, Cold Spring Harbor, N.Y.). In this case, the wash conditions can approach 70-80° C. at 0.1 M NaCl. After washing, the positive colonies can be visualized by exposing the blots to film or to a phosphoimaging screen. The processed image reveals colonies containing cDNA clones of the targeted gene. Alternatively, a non-radioactive method might be chosen to label the probes and detect colony hybridization (for example, the DIG Non-Radioactive DNA Labeling and Detection Kit from Boehringer Mannheim). Each clone is analyzed by restriction digests to identify the longest clone in the group. The longer clones are sequenced using an automated sequencing system (for example a Perkin-Elmer ABI 377) and the sequences evaluated for a complete open reading frame initiated with a methionine start codon (ATG). Genomic clones can also be analyzed for intron-exon boundaries using known methods as well as compared to relevant or homologous gene or protein sequence information to determine the coding regions.

### Example 7

### Expression and Purification of a Polypeptide in E. coli Host

The vector pProEX HT is used for expression of a polypepetide in a bacterial host system. (LifeTechnologies Inc., Gaithersburg, Md.). The plasmid encodes the ampicillin resistance gene (“Apr”) and contains a pBR322 origin of replication (ori), an IPTG inducible promoter, a ribosome binding site, and six codons encoding a histidine tag at the amino terminus. The his tag allows affinity purification using immobilized metal ion affininty chromatography (IMAC), such as with the nickel-nitrilo-tri-acetic acid (Ni-NTA) affinity resin. The cloning region contains suitable restriction enzyme cleavage sites for insertion of polypeptide encoding sequences. The vector also encodes a TEV (Tobbacco Etch Virus) protease cleavage site to remove the his tag region from the amino terminus of the expressed polypeptide.

The desired polypeptide encoding sequence or fragment, typically lacking any hydrophobic leader sequence, is PCR amplified from a cDNA clone. Primers designed from at least one of SEQ NO:1-71, which anneal to the amino terminal sequences of the desired polypeptide encoding region, and from the 3′ end of the cDNA clone, preferably within or at the beginning of vector sequences of the cDNA clone, are used. Additional sequence containing restriction sites, to facilitate cloning into pProEX HT vector, or stop codons can be incorporated into one or both of the primers.

PCR amplified sequences and the vector are digested with appropriate restriction enzymes and then ligated together. Insertion of the DNA into the digested pProEX HT vector places a polypeptide encoding sequence downstream from the trc promoter and in proper reading frame to an initiator AUG.

The ligation mixture is transformed into competent E. coli cells using standard procedures. Sambrook, et al., 1989; Ausubel, 1989, supplements through September 1998. E. coli strain DH5a is used, however other strains are possible. Amp resistant colonies indicate successful transformation. Plasmid DNA from resistant colonies is isolated and the correct construction confirmed by restriction analysis, PCR, and DNA sequencing.

Clones containing the desired constructs are grown over night in LB media supplemented with amp (100 mg/ml). These culture are used to inoculate production cultures, where the cells are grown at 37° C. to an OD590 density of between about 0.4 and 0.6 before induction by adding isopropyl-b-D-thiogalactopyranoside (IPTG) to a final concentration of 1 mM, and allowed an additional 3 to 4 hours to express polypeptide. Cells then are harvested by centrifugation.

The cell pellet is then brought up in 6M guanidine-HCl, pH 8, at 40° C., and stirred for 4 hours. The cell debris is removed by centrifugation and the supernatant containing the expressed polypeptide is dialyzed against a refolding buffer, such as a NaCl-based or Tris-based buffer, at about pH 6 and containing protease inhibitors. After refolding, the polypeptide is purified by IMAC and optionally cleaved with TEV protease. The cleavage reaction is run through a size exclusion gel to purify the desired polypeptide. Purified polypeptide is stored at 4° C. or frozen at −80° C. Gel electrophoresis can be used to verify production of desired polypeptide or for purification.

As noted above, the specific examples should not be interpreted as a limitation to the scope of the invention. Instead, they are merely exemplary embodiments one skilled in the art would understand from the entire disclosure of this invention.

TABLE 1
SEQ ID Correlation Table

| SEQ No. | Clone ID |
|---|---|
| 1 | 004121 |
| 2 | 004845 |
| 3 | 010240 |
| 4 | 043361 |
| 5 | 074351 |
| 6 | 081599 |
| 7 | 101411 |
| 8 | 155796 |
| 9 | 161265 |
| 10 | 215600 |
| 11 | 286367 |
| 12 | 318438 |
| 13 | 318934 |
| 14 | 320704 |
| 15 | 322457 |
| 16 | 323349 |
| 17 | 335737 |
| 18 | 341019 |
| 19 | 341489 |
| 20 | 406288 |
| 21 | 407964 |
| 22 | 420061 |
| 23 | 425402 |
| 24 | 430261 |
| 25 | 430940 |
| 26 | 431066 |
| 27 | 432598 |
| 28 | 446379 |
| 29 | 453896 |
| 30 | 464425 |
| 31 | 483729 |
| 32 | 498992 |
| 33 | 656258 |
| 34 | 777190 |
| 35 | 1220883 |
| 36 | 1241315 |
| 37 | 1243373 |
| 38 | 1262754 |
| 39 | 1298270 |
| 40 | 1391036 |
| 41 | 1485491 |
| 42 | 1488767 |
| 43 | 1535394 |
| 44 | 1581112 |
| 45 | 1675888 |
| 46 | 1746748 |
| 47 | 1824728 |
| 48 | 1867247 |
| 49 | 2195415 |
| 50 | 2432990 |
| 51 | 2434771 |
| 52 | 2436369 |
| 53 | 2436796 |
| 54 | 2437551 |
| 55 | 2438178 |
| 56 | 2438402 |
| 57 | 2438969 |
| 58 | 2441296 |
| 59 | 2441353 |
| 60 | 2441946 |
| 61 | 2443271 |
| 62 | 2491674 |
| 63 | 2641367 |
| 64 | 2687538 |
| 65 | 2868505 |
| 66 | 2933054 |
| 67 | 3115558 |
| 68 | 3209511 |
| 69 | 3326477 |
| 70 | 3340985 |
| 71 | 4592475 |

TABLE 2
Signaling

| SEQ No | Clone ID |
|---|---|
| 8 | 155796 |
| 47 | 1824728 |
| 50 | 2432990 |
| 54 | 2437551 |
| 55 | 2438178 |
| 69 | 3326477 |

TABLE 3
Adhesion

| SEQ No | Clone ID |
|---|---|
| 43 | 1535394 |
| 46 | 1746748 |
| 48 | 1867247 |
| 52 | 2436369 |
| 56 | 2438402 |
| 63 | 2641367 |

TABLE 4
GPCRs

| SEQ No | Clone ID |
|---|---|
| 29 | 453896 |

TABLE 5
Secreted

| SEQ No | Clone ID |
|---|---|
| 3 | 10240 |
| 20 | 406288 |
| 70 | 3340985 |

TABLE 6
Receptors

| SEQ No | Clone ID |
|---|---|
| 19 | 341489 |
| 23 | 425402 |
| 27 | 432598 |
| 38 | 1262754 |
| 39 | 1298270 |
| 41 | 1485491 |
| 44 | 1581112 |
| 57 | 2438969 |
| 58 | 2441296 |
| 60 | 2441946 |
| 61 | 2443271 |
| 62 | 2491674 |
| 65 | 2868505 |
| 67 | 3115558 |
| 68 | 3209511 |

TABLE 7
Product Score 100

| SEQ No | Clone ID |
|---|---|
| 3 | 010240 |
| 8 | 155796 |
| 19 | 341489 |
| 20 | 406288 |
| 23 | 425402 |
| 27 | 432598 |
| 28 | 446379 |
| 29 | 453896 |
| 31 | 483729 |
| 32 | 498992 |
| 34 | 777190 |
| 38 | 1262754 |
| 39 | 1298270 |
| 41 | 1485491 |
| 42 | 1488767 |
| 43 | 1535394 |
| 44 | 1581112 |
| 45 | 1675888 |
| 46 | 1746748 |
| 47 | 1824728 |
| 48 | 1867247 |
| 51 | 2434771 |
| 52 | 2436369 |
| 56 | 2438402 |
| 57 | 2438969 |
| 59 | 2441353 |
| 62 | 2491674 |
| 63 | 2641367 |
| 65 | 2868505 |
| 66 | 2933054 |
| 67 | 3115558 |
| 68 | 3209511 |

TABLE 8
Product Score 50-99

| SEQ No | Clone ID |
|---|---|
| 54 | 2437551 |
| 69 | 3326477 |
| 71 | 4592475 |
| 55 | 2438178 |
| 50 | 2432990 |
| 37 | 1243373 |

TABLE 9
Product Score 0

| SEQ No | Clone ID |
|---|---|
| 1 | 004121 |
| 2 | 004845 |
| 4 | 043361 |
| 5 | 074351 |
| 6 | 081599 |
| 7 | 101411 |
| 9 | 161265 |
| 10 | 215600 |
| 11 | 286367 |
| 12 | 318438 |
| 13 | 318934 |
| 15 | 322457 |
| 16 | 323349 |
| 17 | 335737 |
| 18 | 341019 |
| 21 | 407964 |
| 22 | 420061 |
| 24 | 430261 |
| 25 | 430940 |
| 27 | 431066 |
| 30 | 464425 |
| 33 | 656258 |
| 40 | 1391036 |
| 53 | 2436796 |

TABLE 10
Product Score 1-49

| SEQ No | Clone ID |
|---|---|
| 64 | 2687538 |
| 70 | 3340985 |
| 14 | 320704 |
| 36 | 1241315 |
| 61 | 2443271 |
| 49 | 2195415 |
| 60 | 2441946 |
| 58 | 2441296 |
| 35 | 1220883 |

The clone ID number in the tables above refers to the particular clone in the Incyte database. Each clone ID entry in a table refers to the clone whose sequence is used for (1) the sequence comparison whose scores are presented and/or (2) assignment to the particular cluster which is presented. Note that a clone may be included in this table even if its sequence comparison scores fail to meet the minimum standards for similarity. In such a case, the clone is included due solely to its association with a particular cluster for which sequences of one or more other member clones possess the required level of similarity.

### REFERENCES

All references, patents, or applications cited herein are incorporated by reference in their entirety, as if written herein.

Antica et al., Blood 84:111-117 (1994)

Armour, et al., FEBS Lett. 307:113-115 (1992)

Ausubel, et al., Current Protocols in Molecular Biology, John Wiley & Sons, N.Y. (1989) and supplements through September 1998

Bachem et al., Plant J. 9:745-753 (1996)

Bains and Smith, J. Theor. Biol. 135:303 (1989)

Baldwin et al., Gene Ther. 4:1142-1149 (1997)

Barany, Proc. Natl. Acad. Sci. (U.S.A.) 88:189-193 (1991)

Bassat et al., J. Bac. 169:751-757, 1987

Becker et al., Mol. Gen. Genet. 249:65-73 (1995)

Beismann et al., Mol. Ecol. 6:989-993 (1997)

Benkel et al., Genet. Anal. 13:123-127 (1996)

Bennett, et al., Measurement of human Interleukin 11, In Current Protocols in Immunology. Coligan eds. Vol 1 pp. 6.15.1 John Wiley and Sons, Toronto (1991)

Bertagnolli et al., Cellular Immunology 133:327-341 (1991)

Bertagnolli et al., J. Immunol. 145:1706-1712 (1990)

Bertagnolli et al., J. Immunol. 149:3778-3783 (1992)

Bhardwaj et al., Journal of Clinical Investigation 94:797-807 (1994)

Bierer et al., J. Exp. Med. 168:1145-1156 (1988)

Birren et al., Genome Analysis: Analyzing DNA, 1, (1997), Cold Spring Harbor Laboratory Press, Cold Spring Harbor, N.Y.

Bishop and Posse, Adv. Gene Technol. 1:55 (1990)

Bolivar et al., Gene 2:95 (1977)

Botstein, et al., Ann. J. Hum. Genet. 32:314-331 (1980)

Bowman et al., J. Immunol. 152:1756-1761 (1994)

Bowman et al., J. Virology 61:1992-1998

Branco Ferreiraand Palmo Carlos, Cytokines and Asthma, Invest. Allergol. Clin. Immunol. 8:141-148, (1998)

Brown et al., J. Immunol. 153:3079-3092, 1994

Chakrabarti et al., Mol. Cell. Biol. 5:3403 (1985)

Chang et al., Nature 275:615 (1978)

Chen and Kwok, Nucl. Acids Res. 25:347-353 (1997)

Cho et al., Genome 39:373-378 (1996)

Chung and Miller, Nucleic Acids Res. 16:3580 (1988)

Ciarletta, et al., Measurement of mouse and human Interleukin 9,, In Current Protocols in Immunology. Coligan eds. Vol 1 pp. 6.13.1, John Wiley and Sons, Toronto (1991).

Cnops et al., Mol. Gen. Genet. 253:32-41 (1996)

Coligan, ed. Current Protocols in Immunology, John Wiley and Sons, Toronto (1994) (Chapter 3, In Vitro assays for Mouse Lymphocyte Function; Chapter 6, Cytokines and their cellular receptors; Chapter 7, Immunologic studies in Humans)

Coulson, Trends in Biotechnology 12:76-80 (1994)

Darzynkiewicz et al., Cytometry 13:795-808 (1992)

deBoer et al., Proc. Natl. Acad. Sci. USA 80:21-25 (1983

deVries et al., J. Exp. Med. 173:1205-1211 (1991)

Doerfler, Curr. Top. Microbiol. Immunol. 131:51-68 (1968)

Eaglstein and Mertz, J. Invest. Dermatol 71:382-84 (1978). Coligan et al., eds., Current Protocols in Immunology, Pub. Greene Publishing Associates and Wiley-Interscience (Chapter 6.12, Measurement of alpha and beta Chemokines 6.12.1-6.12.28)

Elles, Methods in Molecular Medicine: Molecular Diagnosis of Genetic Diseases, Humana Press (1996)

Elshami et al., Cancer Gene Ther. 4:213-221 (1997)

Fiers et al., Nature 273:113 (1978)

Fine et al., Cellular Immunology 155:111-122 (1994)

Fodor et al., Science 251:767-773 (1991)

Folkertsma et al., Mol. Plant Microbe Interact. 9:47-54 (1996

Frasier, In Vitro Cell. Dev. Biol. 25:225 (1989)

Freshney, Methylcellulose colony forming assays, In Culture of Hematopoietic Cells. Freshney, et al., eds. Vol pp. 265-268, Wiley-Liss, Inc., New York, N.Y. (1994)

Frohman et al., Proc. Natl. Acad. Sci. (U.S.A.) 85:8998-9002 (1988)

Galy et al., Blood 85:2770-2778 (1995)

Game et al., Analytical Biochemistry 258:127 (1998)

Goeddel et al., Nature 281:544 (1979)

Goeddel, Nucleic Acids Res. 8:4057 (1980)

Gorczyca et al., Cancer Research 53:1945-1951 (1993)

Gorczyca et al., International Journal of Oncology 1:639-648 (1992)

Gorczyca et al., Leukemia 7:659-670 (1993)

Gray et al., Proc. R. Acad. Soc. Lond. 243:241-253 (1991)

Greenberger et al., Proc. Natl. Acad. Sci. (U.S.A.) 80:2931-2938 (1983)

Gruber et al., J. of Immunol. 152:5860-5867 (1994)

Guarino and Summers, J. Virol. 61:2091-2099 (1987)

Guarino and Summers, J. Virol. 57:563-571 (1986)

Guarino and Summers, Virol. 162:444-451 (1988)

Guery et al., J. Immunol. 134:536-544, 1995

Gusella, Ann. Rev. Biochem. 55:831-854 (1986)

Guzman et al., J. Bacteriol. 174:7716-7728 (1992)

Haff and Smirnov, Genome Res. 7: 378-388 (1997)

Handa et al., J. Immunol. 135:1564-1572 (1985)

Harlow and Lane, Antibodies: A Laboratory Manual, Cold Spring Harbor Press, Cold Spring Harbor, N.Y. (1988)

Harms and Splitter, Hum. Gene Ther. 6:1291-1297 (1995)

Hartl et al., Methods Mol. Biol. 58:293-301 (1996)

Haymes, et al. Nucleic Acid Hybridization, A Practical Approach, IRL Press, Washington, D.C. (1985)

Herrmann et al., J. Immunol. 128:1968-1974 (1982)

Herrmann et al., Proc. Natl. Acad. Sci. (U.S.A.) 78:2488-2492 (1981)

Hillel et al., Anim. Genet. 20:145-155 (1989)

Hillel et al., Genet. 124:783-789 (1990)

Hirayama et al., Proc. Natl. Acad. Sci. (U.S.A.) 89:5907-5911 (1992)

Huang et al., Methods Mol. Biol. 69: 89-96 (1997)

Huang et al., Science 264:961-965 (1994)

Huang, et al., Method Mol. Biol. 67:287-294 (1997)

Huys et al., Int. J. Syst. Bacteriol. 46:572-580 (1996)

Huys et al., Int. J. Syst. Bacteriol. 47:1165-1171 (1997)

Inaba et al., Journal of Experimental Medicine 172:631-640 (1990)

Inaba et al., Journal of Experimental Medicine 173:549-559, (1991)

Itoh et al., Cell 66:233-243 (1991)

Janknecht et al., Carcinogenesis 16:443-450 (1995)

Janknecht, Immunobiology 193:137-142 (1995)

Janssen et al., Int. J. Syst. Bacteriol. 47:1179-1187 (1997)

Jeffreys et al., Amer. J. Hum. Genet. 39:11-24(1986)

Jeffreys et al., Anim. Genet. 18:1-15 (1987)

Jeffreys et al., Nature 316:76-79 (1985)

Johansson et al., Cellular Biology 15:141-151 (1995)

Johnston et al. J. of Immunol. 153:1762-1768 (1994)

Jones, et al., Eur. J. Haematol. 39:144-147 (1987)

Kay et al., Science 236:1299 (1987) Keim et al., J. Bacteriol. 179:818-824 (1997)

Keller et al., Molecular and Cellular Biology 13:473-486 (1993)

Keown et al., Methods Enzymol. (1989)

Keown et al., Methods Enzymol. 185:527-537 (1990)

Konieczny and Ausubel, Plant J. 4:403-410 (1993)

Kruisbeek, and Shevach, In Current Protocols in Immunology, Coligan eds. Vol 1 pp. 3.12.1-3.12.14, John Wiley and Sons, Toronto (1994)

Krzesicki, et al., Am. J. Respir. Cell Mol. Biol. 16:693-701 (1997)

Kuppuswami et al., Proc. Natl. Acad. Sci. USA 88:1143-1147 (1991)

Kwoh, et al., Proc. Natl. Acad. Sci. (U.S.A.) 86:1173 (1989)

Labrune et al., Am. J. Hum. Genet. 48:1115-1120 (1991)

Landegren, et al., Science 241:1077-1080 (1988)

Latorra et al., PCR Methods Appl. 3:351-358 (1994)

Lee et al., Anal. Biochem. 205:289-293 (1992)

Lin et al., Nucleic Acids Res. 24:3649-3650 (1996)

Lind et al., APMIS 103:140-146 (1995)

Livak et al., Nature Genet. 9:341-342 (1995)

Livak et al., PCR Methods Appl. 4:357-362 (1995a)

Lo et al., Nucleic Acids Research 20:1005-1009 (1992)

Lopez-Rodriguez, Leuk. Lymphoma 25:415-425 (1997)

Luckow and Summers, Bio/Technology 6:47-55 (1988a)

Macatonia et al., J Immunol 154:5071-5079 (1995)

Macatonia et al., Journal of Experimental Medicine 169:1255-1264 (1989)

Mackett et al, J Virol. 49:857 (1984)

Maliszewski, J. Immunol. 144:3028:3033 (1990)

Mansour et al., Nature 336:348-352, (1988)

McClanahan et al., 81:2903-2915 (1993)

McCouch et al., Plant Mol. Biol. 35:89-99 (1997)

McNiece, and Briddell, In Culture of Hematopoietic Cells, Freshney, et al. eds. Vol pp. 23-39, Wiley-Liss, Inc., New York, N.Y. (1994)

Meksem et al., Mol. Gen. Genet. 249:74-81 (1995)

Miller, Annual Review of Microbiol. 42:177-199 (1988)

Mond, and Brunswick, Assays for B cell function: In vitro antibody production, In Current Protocols in Immunology. Coligan eds. Vol 1 pp. 3.8.1-3.8.16, John Wiley and Sons, Toronto (1994)

Money et al., Nucleic Acids Res. 24:2616-2617 (1996)

Moore et al., Genomics 10:654-660 (1991)

Moreau et al., Nature 336:690-692 (1988)

Mori and Prager, Leuk. Lymphoma 26:421-433 (1997)

Moss, In: Gene Transfer Vectors For Mammalian Cells (Miller and Calos, eds., Cold Spring Harbor Laboratory, N.Y., p. 10, (1987)

Muller et al., Eur. J. Immunol. 25:1744-1748

Myers et al., Nature 313:495-498 (1985)

Nair et al., J. Virology 67:4062-4069 (1993)

Nandi et al., Mol. Gen. Genet. 255:1-8 (1997

Neben et al., Experimental Hematology 22:353-359 (1994)

Neff et al., Plant J. 14:387-392 (1998)

Newton et al., Nucl. Acids Res. 17:2503-2516 (1989)

Nickerson, et al., Proc. Natl. Acad. Sci. (U.S.A.) 87:8923-8927 (1990)

Nikiforov et al., Nucl. Acids Res. 22:4167-4175 (1994)

Nordan, R. Measurement of mouse and human interleukin 6, In Current Protocols in Immunology. Coligan eds. Vol 1 pp. 6.6.1-6.6.5, John Wiley and Sons, Toronto (1994)

Norman et al., Vaccine 15:801-803 (1997)

O'Neill et al., Transplant Proc. 23:2862-2866 (1991)

Obukowicz et al., Applied Environmental Microbiology 58:1511-1523, (1992)

Ohara et al., Proc. Natl. Acad. Sci. (U.S.A.) 86:5673-5677 (1989)

Orita et al., Genomics 5:874-879 (1989)

Pang et al., Biotechniques 22(6):1046-1048 (1977)

Pesole, et al., BioTechniques 25:112-123 (1998)

Ploemacher, In Culture of Hematopoietic Cells, Freshney, et al., eds. Vol pp. 1-21, Wiley-Liss, Inc., New York, N.Y. (1994)

Porgador et al., Journal of Experimental Medicine 182:255-260 (1995)

Prober et al., Science 238:336-340 (1987)

Qi et al., Mol. Gen. Genet. 254:330-336 (1997)

Rachal et al., EXS 64:330-342 (1993)

Ray et al., Adv. Exp. Med. Biol. 280:107-111 (1990)

Rosenstein et al., J. Exp. Med. 169:149-160 (1989)

Sambrook et al., Molecular Cloning, A Laboratory Manual 2nd Ed., Cold Spring Harbor Press, Cold Spring Harbor, N.Y. (1989)

Sarkar et al., Genomics 13:441-443 (1992)

Savvas, C. M., Microbiological Reviews 60:512-538 (1996)

Schena et al., Science 270:467-470 (1995)

Schreiber, Measurement of mouse and human Interferon gamma, In Current Protocols in Immunology, Coligan eds. Vol 1 pp. 6.8.1-6.8.8, John Wiley and Sons, Toronto (1994). Bottomly, Davis, Measurement of Human and Murine Interleukin 2 and Interleukin 4 Lipsky In Current Protocols in Immunology. Coligan eds. Vol 1 pp. 6.3.1-6.3.12, John Wiley and Sons, Toronto (1994)

Serafin, 1995 Current Protocols in Molecular Biology

Serafin, Drugs used in the treatment of asthma, In Hardman, and Limbird, eds. Goodman and Gilman's, The Pharmacological Basis of Therapeutics, 9th ed., pp. 659-682 (1995)

Serfing et al., Biochim. Biophys. Acta 1263:181-200 (1995)

Shalon, Ph.D. Thesis, Stanford University (1996)

Shannon et al., Crit. Rev. Immunol. 17:301-323 (1997)

Siebenlist et al., Cell 20:269 (1980)

Simons et al., Genomics 44:61-70 (1997)

Skolnick, et al., Cytogen. Cell Genet. 32:58-67 (1982)

Smith et al., Proc. Natl. Acad. Sci. (U.S.A.) 83:1857-1861 (1986)

Spooncer, et al., In Culture of Hematopoietic Cells, Freshney, et al., eds. Vol pp. 163-179, Wiley-Liss, Inc., New York, N.Y. (1994)

Stitt et al., Cell 80:661-670 (1995).

Stoltenborg et al., J. Immunol. Methods 175:59-68, 1994

Summers and Smith, A Manual of Methods for Baculovirus Vectors and Insect Cell Culture Procedures, Texas Agricultural Experiment Station Bulletin No. 1555, Texas A&M University (1987)

Summers, Curr. Comm. Molecular Biology, Cold Spring Harbor Press, Cold Spring Harbor, N.Y. (1988)

Sun et al., Curr. Top. Microbiol. Immunol 211:173-187 (1996)

Sutherland, In Culture of Hematopoietic Cells, R. I. Freshney, et al., eds. Vol pp. 139-162, Wiley-Liss, Inc., New York, N.Y. (1994)

Suzuki et al., Anal. Biochem. 192:82-84 (1991)

Taillon-Miller et al. Genome Res. 8:748-754 (1998)

Takai et al., J. Immunol. 137:3494-3500 (1986)

Takai et al., J. Immunol. 140:508-512 (1988)

Takai et al., Princess Takamatsu Symp. 22:197-204 (1991)

Takai et al., Proc. Natl. Acad. Sci. (U.S.A.) 84:6864-6868 (1987)

Taub et al., J. Clin. Invest. 95:1370-1376 (1995)

Thomas et al., Plant J. 8:785-794 (1995)

Toki et al., Proc. Nat. Acad Sci. (U.S.A.) 88:7548-7551 (1991)

Tong et al., Anticancer Res. 18:719-725 (1998)

Travis et al., Mol. Ecol. 5:735-745 (1996)

Tyagi et al., Nature Biotech. 16:49-53 (1998)

Van der Lee et al., Fungal Genet. Biol. 21:278-291 (1997)

Van der Voort et al., Mol. Gen. Genet. 255:438-447 (1997

Van Heeke and Schuster J. Biol. Chem. 264:5503-5509 (1989)

Volkman and Summers, J. Virol 19: 820-832 (1975)

Volkman et al., J. Virol 19:820-832 (1976)

Vos et al., Nucleic Acids Res. 23:4407-4414 (1995)

Walker, et al., Proc. Natl. Acad. Sci. (U.S.A.) 89:392-396 (1992)

Waugh et al., Mol. Gen. Genet. 255:311-321 (1997)

Webb and Summers, Technique 2:173 (1990)

Weinberg et al., Gene 126: 25-33 (1993)

Weinberger et al., Eur. J. Immun. 11:405-411 (1981)

Weinberger et al., Proc. Natl. Acad. Sci. (U.S.A) 77:6091-6095 (1980)

Winter, Epidermal Wound Healing, pps. 71-112 (Maibach, and Rovee, eds.), Year Book Medical Publishers, Inc., Chicago

Wu et al., Proc. Natl. Acad. Sci. USA 86:2757-2760 (1989)

Wu, et al., Genomics 4:560 (1989)

Yanish-Perron et al. Gene 33:103-119 (1985)

Yoshiuchi et al., J. Clin. Endocrin. Metab. 83:1016-1019 (1998)

Zacharchuk, J. Immunol. 145:4037-4045 (1990)

Zamai et al., Cytometry 14:891-897 (1993)

Patents

Albarella et al., EP 144914;

Albarella et al., U.S. Pat. No. 4,563,417

Burn et al., U.S. Pat. No. 5,556,954

Davey, et al., EP Application 329,822

EP 36,776

Fischer, et al. (PCT Application WO90/13668)

Gingeras, et al., PCT application WO 88/10315

Glassberg, UK Patent Application 2135774

Horn, et al., PCT Patent Application WO91/14003

International Patent Publication No. WO91/07491

International Patent Publication No. WO95/05846

International Patent Publication No. WO95/16035

Jeffreys, European Patent Application 370,719

Jeffreys, U.S. Pat. No. 5,175,082

Malek, et al., U.S. Pat. No. 5,130,238

Miller, et al., PCT appln. WO 89/06700

Miyoshi et al., EP 119448

Schuster et al., U.S. Pat. No. 5,169,766

Sheldon et al., U.S. Pat. No. 4,582,789

Smith and Summers, U.S. Pat. No. 4,745,051

Summers, U.S. Pat. No. 5,155,037

Summers, U.S. Pat. No. 5,278,050

U.S. Pat. No. 4,923,901

U.S. Pat. No. 5,079,600

U.S. Pat. No. 5,143,854

U.S. Pat. No. 5,304,472

U.S. Pat. No. 5,342,763

U.S. Pat. No. 5,445,934

U.S. Pat. No. 5,759,787

U.S. Pat. No. 4,518,584

U.S. Pat. No. 4,757,006

U.S. Pat. No. 5,800,992

Uhlen, PCT Application WO90/11369

Weber, U.S. Pat. No. 5,075,217

We claim:

1. A substantially-purified nucleic acid having a nucleotide sequence selected from the group consisting of SEQ NO: 1 through SEQ NO: 71, or complements thereof.

2. A substantially-purified nucleic acid capable of specifically hybridizing to a nucleic acid as claimed in claim 1, or a complement thereof.

3. A substantially-purified nucleic acid exhibiting a percentage identity of between about 90% to about 99% with at least a 10 nucleotide region of the sequence of a nucleic acid as claimed in claim 1.

4. A substantially-purified nucleic acid exhibiting a percentage identity of between about 70% to about 90% with at least a 10 nucleotide region of the sequence of a nucleic acid as claimed in claim 1.

5. A substantially-purified nucleic acid as claimed in any one of claims 3 or 4 that is capable of being used as a hybridization probe or PCR probe.

6. A method of identifying a nucleic acid comprising contacting a hybridization probe as claimed in claim 5 with a sample containing nucleic acid and detecting hybridization to the hybridization probe.

7. A method of identifying a nucleic acid comprising contacting a PCR probe as claimed in claim 5 with a sample containing nucleic acid and producing multiple copies of a nucleic acid that hybridizes to the PCR probe.

8. A substantially-purified nucleic acid having at least one 10 nucleotide region substantially identical to a sequence identified in Table 1.

9. A substantially-purified nucleic acid of claim 8, wherein said nucleic acid encodes a portion of a polypeptide having an activity selected from the group consisting of G-protein coupled receptor binding activity, kinase activity, protease activity, cell adhesion activity, and cytotoxic activity.

10. A nucleic acid of claim 9 wherein said activity is G protein coupled receptor binding activity.

11. A nucleic acid of claim 9 wherein said activity is kinase activity.

12. A nucleic acid of claim 9 wherein said activity is protease activity.

13. A nucleic acid of claim 9 wherein said activity is cell adhesion activity.

14. A nucleic acid of claim 9 wherein said activity is cytotoxic activity.

15. A recombinant DNA comprising a nucleic acid according to one of claims 1-4, or 8, wherein the recombinant nucleic acid further comprises a promoter or partial promoter region.

16. A cell containing a nucleic acid as claimed in claim 15.

17. A substantially-purified protein, polypeptide, or fragment thereof, wherein at least one 15 amino acid region is encoded by a nucleic acid as claimed in one of claims 1-4, or 8.

18. A substantially-purified protein, polypeptide, or fragment thereof as claimed in claim 17, which possesses an activity selected from the group consisting of G protein coupled receptor binding activity, kinase activity, protease activity, and cell adhesion activity, and cytotoxic activity.

19. A substantially-purified protein, polypeptide, or fragment thereof as claimed in claim 18, which possesses G protein coupled receptor binding activity.

20. A substantially-purified protein, polypeptide, or fragment thereof as claimed in claim 18, which possesses kinase activity.

21. A substantially-purified protein, polypeptide, or fragment thereof as claimed in claim 18, which possesses protease activity.

22. A substantially-purified protein, polypeptide, or fragment thereof as claimed in claim 18, which possesses cell adhesion activity.

23. A substantially-purified protein, polypeptide, or fragment thereof as claimed in claim 18, which possesses cytotoxic activity.

24. An antibody that specifically binds to a purified protein, polypeptide, or fragment thereof, having at least one region of 5 contiguous amino acids encoded by a nucleic acid as claimed in one of claims 1-4 or 8.

25. A transgenic animal having in one or more of its cells an introduced nucleic acid as claimed in one of claims 1-4 or 8, or progeny of the transgenic animal.

26. A cell taken from a transgenic animal or its progeny as claimed in claim 25.

27. A composition comprising a nucleic acid as claimed in one of claims 1-3, or a complement thereof.

28. A method of identifying a biologically active compound or composition comprising contacting the compound or composition with a sample comprising a protein, polypeptide, or fragment as claimed in one of claims 17, and comparing the interaction between the compound or composition and the protein, polypeptide, or fragment with a control.

29. A compound or composition that is detectable in a method of claim 28.

30. A computer-readable medium having recorded thereon the sequence information of one or more of SEQ NO:1 through SEQ NO: 71 or complements thereof.

31. A method of identifying a nucleic acid comprising providing a computer-readable medium as claimed in claim 30 and comparing nucleotide sequence information using a computerized means.

32. A substantially-purified nucleic acid molecule which comprises a nucleic acid sequence that is identical to at least 20 nucleotides of a nucleotide sequence selected from the group consisting of SEQ NO: 1 through SEQ NO: 71, or complements thereof.

33. A substantially-purified nucleic acid molecule which comprises a nucleic acid sequence that is identical to at least 50 nucleotides of a nucleotide sequence selected from the group consisting of SEQ NO: 1 through SEQ NO: 71, or complements thereof.

34. A substantially-purified nucleic acid molecule which comprises a nucleic acid sequence that is identical to at least 100 nucleotides of a nucleotide sequence selected from the group consisting of SEQ NO: 1 through SEQ NO: 71, or complements thereof.

35. A substantially-purified protein, polypeptide, or fragment thereof, of claim 17 wherein said substantially-purified protein, polypeptide, or fragment thereof is a fusion protein.

36. An antibody of claim 24 that is detectably-labeled.

37. A transformed cell having a nucleic acid molecule of claim 1.

38. A transformed cell having the antisense of a nucleic acid molecule of claim 1.

39. A process for diagnosis or prognosis of asthma in a mammal from the expression of mRNA or cDNA that is identical to at least 20 nucleotides of a nucleotide sequence selected from the group consisting of SEQ NO: 1 through SEQ NO: 71, or complements thereof.

40. A method of isolating a nucleic acid that is identical to at least 20 nucleotides of a nucleotide sequence selected from the group consisting of SEQ NO: 1 through SEQ NO: 71, or complements thereof.

41. A substantially-purified nucleic acid as described in Table 7 having a product score of 100.

42. A substantially-purified nucleic acid as described in Table 8 having a product score between 50 and 99.

43. A substantially-purified nucleic acid as described in Table 9 having a product score of 0.

44. A substantially-purified nucleic acid as described in Table 10 having a product score between 1 and 49.

45. A method of identifying a biologically active compound or composition comprising contacting the compound or composition with a sample comprising a protein, polypeptide, or fragment as claimed in claim 18, and comparing the interaction between the compound or composition and the protein, polypeptide, or fragment with a control.

46. A compound or composition that is detectable in a method of claim 45.

47. A method of identifying a biologically active compound or composition comprising contacting the compound or composition with a sample comprising a protein, polypeptide, or fragment as claimed in claim 19, and comparing the interaction between the compound or composition and the protein, polypeptide, or fragment with a control.

48. A compound or composition that is detectable in a method of claim 47.

49. A method of identifying a biologically active compound or composition comprising contacting the compound or composition with a sample comprising a protein, polypeptide, or fragment as claimed in claim 20, and comparing the interaction between the compound or composition and the protein, polypeptide, or fragment with a control.

50. A compound or composition that is detectable in a method of claim 49.

51. A method of identifying a biologically active compound or composition comprising contacting the compound or composition with a sample comprising a protein, polypeptide, or fragment as claimed in claims 21, and comparing the interaction between the compound or composition and the protein, polypeptide, or fragment with a control.

52. A compound or composition that is detectable in a method of claim 51.

53. A method of identifying a biologically active compound or composition comprising contacting the compound or composition with a sample comprising a protein, polypeptide, or fragment as claimed in claim 22, and comparing the interaction between the compound or composition and the protein, polypeptide, or fragment with a control.

54. A compound or composition that is detectable in a method of claim 53.

55. A method of identifying a biologically active compound or composition comprising contacting the compound or composition with a sample comprising a protein, polypeptide, or fragment as claimed in claim 23, and comparing the interaction between the compound or composition and the protein, polypeptide, or fragment with a control.

56. A compound or composition that is detectable in a method of claim 55.
